(19) United States
(12) Patent Application Publication
(10) Pub. No.: US 2024/0390495 A1
(43) Pub. Date: Nov. 28, 2024

(54) CHIMERIC RECEPTOR POLYPEPTIDES IN COMBINATION WITH TRANS METABOLISM MOLECULES THAT RE-DIRECT GLUCOSE METABOLITES OUT OF THE GLYCOLYSIS PATHWAY AND THERAPEUTIC USES THEREOF

(71) Applicant: SOTIO BIOTECH INC., Boston, MA (US)

(72) Inventors: Kathleen MCGINNESS, Boston, MA (US); Seth ETTENBERG, Boston, MA (US); Luke BARRON, Boston, MA (US); Michael FRAY, Boston, MA (US); Charles WILSON, Boston, MA (US); Gregory MOTZ, Boston, MA (US); Samyabrata BHADURI, Boston, MA (US); Amy JENSEN, Boston, MA (US); Emily KUIPER, Boston, MA (US)

(73) Assignee: SOTIO BIOTECH INC., Boston, MA (US)

(21) Appl. No.: 18/695,383

(22) PCT Filed: Sep. 27, 2022
(86) PCT No.: PCT/US2022/077103

§ 371 Date: Mar. 26, 2024

Related U.S. Application Data
(60) Provisional application No. 63/399,324, filed on Aug. 19, 2022.
(60) Provisional application No. 63/248,629, filed on Sep. 27, 2021.

(51) Int. Cl. A61K 39/00; A61P 35/00; C07K 14/705; C07K 14/725; C07K 14/735; C07K 16/30; C12N 5/0783

(52) U.S. Cl. CPC A61K 39/4631; A61K 39/4611; A61K 39/4613; A61K 39/4644; A61P 35/00; C07K 14/7051; C07K 14/70517; C07K 14/70521; C07K 14/70535; C07K 16/30; C12N 5/0636; C12N 5/0646; C07K 2319/02; C07K 2319/03; C12N 2510/00

(57) ABSTRACT

Genetically engineered hematopoietic cells, which express one or more factors that redirect glucose metabolites, and optionally a chimeric receptor polypeptide (e.g., an antibody-coupled T cell receptor (ACTR) polypeptide, a chimeric antigen receptor (CAR) polypeptide, or a TCR polypeptide) capable of binding to a target antigen of interest. Also disclosed herein are uses of the engineered hematopoietic cells for inhibiting cells expressing a target antigen in a subject in need thereof.

### CROSS REFERENCE TO RELATED APPLICATIONS

This application claims the benefit of the filing dates of U.S. Provisional Application No. 63/248,629, filed Sep. 27, 2021, and U.S. Provisional Application No. 63/399,324, filed Aug. 19, 2022, the entire contents of each of which are incorporated by reference herein.

### FIELD OF THE INVENTION

The present invention relates to a genetically modified immune cells expressing a Chimeric Receptor polypeptide (e.g., Chimeric Antigen Receptor, CAR) and a factor that re-direct metabolites out of a metabolism pathway. The present invention further relates to a CAR-NK or CAR-T and its use in particular for treating cancer.

### BACKGROUND OF DISCLOSURE

Cancer immunotherapy, including cell-based therapy, is used to provoke immune responses attacking tumor cells while sparing normal tissues. It is a promising option for treating various types of cancer because of its potential to evade genetic and cellular mechanisms of drug resistance, and to target tumor cells while sparing normal tissues.

Cell-based therapy may involve cytotoxic T cells having reactivity skewed toward cancer cells (Eshhar et al., Proc Natl Acad Sci USA, 90(2): 720-724 (1993); Geiger et al., The Journal of Immunology, 162(10): 5931-5939 (1999); Brentjens et al., Nat Med, 9(3): 279-286 (2003); Cooper et al., Blood, 101(4): 1637-1644 (2003); Imai et al., Leukemia, 18(4): 676-684 (2004)). While cell-based immune therapies have shown promising therapeutic effects, they have faced challenges caused by specific characteristics of the tumor microenvironment (TME), which is cellular environment created via the interaction between malignant tumor cells and non-transformed cells.

It is therefore of great importance to develop strategies to improve efficacy of cell-based immune therapies in light of the TME.

### SUMMARY OF DISCLOSURE

The present disclosure is based on the development of strategies to divert or re-direct glucose metabolites out of the glycolysis pathway in hematopoietic cells such as immune cells, including those that express a chimeric receptor polypeptide, such as an antibody-coupled T-cells receptor (ACTR) polypeptide, chimeric antigen receptor (CAR) polypeptide or a T Cell Receptor (TCR) polypeptide, for use in cell-based immune therapy. Re-direction of glucose metabolites out of the glycolysis pathway may be achieved by expressing (e.g., over-expressing) in hematopoietic stem cells (HSCs), preferably immune cells (e.g., αβ or γδ T cells or NK cells), one or more factors (e.g., proteins or nucleic acids) such as those described herein. Such genetically engineered immune cells are expected to have an enhanced metabolic activity relative to native hematopoietic cells of the same type, for example, in a low glucose, low amino acid, low pH, and/or hypoxic environment (e.g., in the TME). As such, hematopoietic cells such as HSCs or immune cells that co-express one or more factors (e.g., polypeptides or nucleic acids) that redirect glucose metabolites out of the glycolysis pathway in the hematopoietic cells and a chimeric receptor polypeptide would exhibit superior bioactivities (e.g., under low glucose, low amino acid, low pH, and/or hypoxic conditions), for example, cell survival, cell proliferation, maintaining its activated phenotype (e.g., increased cytokine production, e.g., IL-2 or IFN-γ production), cytotoxicity, and/or in vivo anti-tumor activity.

Accordingly, provided herein are modified (e.g., genetically engineered) hematopoietic cells (e.g., HSCs, preferably immune cells (e.g., αβ or γδ T cells, or NK cells) that have altered intracellular regulation of glucose concentrations relative to the wild-type immune cells of the same type. In some instances, the modified immune cells may express or overly express a factor that redirects glucose metabolites, for example, a polypeptide that diverts or redirects glucose metabolites out of the glycolysis pathway. A modified immune cell expressing any of the factor that redirects glucose metabolite refers to a genetically engineered immune cell into which an exogenous nucleic acid encoding the factor is introduced such that the encoded factor is expressed in the resultant modified immune cell, while the unmodified parent cell does not express such a factor. A modified immune cell overly expressing any of the factor that redirects glucose metabolite refers to a genetically engineered immune cell, which is engineered to enhance the expression level of the factor as relative to the unmodified parent cell. In some instances, the modified immune cell may be engineered to enhance expression of the endogenous gene encoding the factor. Alternatively, the modified immune cell may be engineered to transfect an exogenous nucleic acid encoding the factor for producing additional amount of the factor in the modified immune cell.

The factor that redirects glucose metabolites may divert or redirect substrates out of the glycolysis pathway directly (e.g., glutamine-fructose-6-phosphate aminotransferase 1 (GFPT1)) or indirectly by, for example, decreasing the rate of glucose breakdown in the glycolysis pathway (e.g., TP53-inducible glycolysis and apoptosis regulator (TIGAR), pyruvate kinase muscle isozyme M2 (PKM2), of PKM2 variants). Exemplary polypeptides that redirect glucose metabolites out of the glycolysis pathway include, but are not limited to, pyruvate kinase muscle isozyme M2 (PKM2), glutamine-fructose-6-phosphate aminotransferase 1 (GFPT1), TP53-inducible glycolysis and apoptosis regulator (TIGAR), and functional variants thereof (e.g., PKM2 Y105E, PKM2 Y105D, PKM2 K422R, and PKM2 H391Y). In specific examples, the polypeptide that diverts or directs glucose metabolites is TIGAR.

The modified immune cells may further express a chimeric receptor polypeptide, which may comprise (a) an extracellular target binding domain; (b) a transmembrane domain; and (c) a cytoplasmic signaling domain (e.g., a cytoplasmic domain that comprises an immunoreceptor tyrosine-based activation motif (ITAM)). In one embodiment, a genetically engineered immune cell, which has altered glucose metabolism as compared with a native immune cell of the same type, wherein the immune cell: (i) expresses or overly expresses a polypeptide that diverts or redirects glucose metabolites out of a glycolysis pathway, and (ii) a chimeric receptor polypeptide; wherein the chimeric receptor polypeptide comprising (a) an extracellular target binding domain; (b) a transmembrane domain; and (c) a cytoplasmic signaling domain. Any of the chimeric polypeptides disclosed herein may further comprise at least one co-stimulatory signaling domain. In other embodiments, the chimeric receptor polypeptide may be free of co-stimulatory signaling domains.

In some embodiments, the chimeric receptor polypeptide is an antibody-coupled T cell receptor (ACTR), which comprises an extracellular Fc-binding domain (a). In other embodiments, the chimeric receptor is a chimeric antigen receptor (CAR), which comprises an extracellular antigen binding domain (a). Alternatively or in addition, the cytoplasmic signaling domain (c) is located at the C-terminus of the chimeric receptor polypeptide. In some embodiments, the chimeric receptor polypeptide is a T cell receptor (TCR), which comprises an extracellular domain, or portion thereof, of a TCR α chain, a TCR β chain, a TCR γ chain, a TCR δ chain, a CD3 ε TCR subunit, a CD3 γ TCR subunit, a CD3 δ TCR subunit, or a CD3z TCR subunit.

In some embodiments, the chimeric receptor polypeptides described herein (e.g., an ACTR polypeptide, a CAR, or a TCR polypeptide) may further comprise a hinge domain, which is located at the C-terminus of (a) and the N-terminus of (b). In other embodiments, the chimeric receptor polypeptide may be free of any hinge domain. In yet other embodiment, the chimeric receptor polypeptide, for example, an ACTR polypeptide, may be free of a hinge domain from any non-CD16A receptor. Alternatively, or in addition, the chimeric receptor polypeptide further comprises a signal peptide at its N-terminus. Further in another embodiment, the cytoplasmic signaling domain (c) comprises an immunoreceptor tyrosine-based activation motif (ITAM).

In some embodiments, the chimeric receptor polypeptide disclosed herein may be an ACTR polypeptide comprising an Fc binding domain (a). In some examples, the Fc binding domain of (a) can be an extracellular ligand-binding domain of an Fc-receptor, for example, an extracellular ligand-binding domain of an Fc-gamma receptor, an Fc-alpha receptor, or an Fc-epsilon receptor. In particular examples, the Fc binding domain is an extracellular ligand-binding domain of CD16A (e.g., F158 CD16A or V158 CD16A), CD32A, or CD64A. In other examples, the Fc binding domain of (a) can be an antibody fragment that binds the Fc portion of an immunoglobulin. For example, the antibody fragment can be a single chain variable fragment (ScFv), a single domain antibody, (e.g., a nanobody). Additionally, the Fc binding domain of (a) can be a naturally-occurring protein that binds the Fc portion of an immunoglobulin or an Fc-binding fragment thereof. For example, the Fc binding domain can be Protein A or Protein G, or an Fc-binding fragment thereof. In further examples, the Fc binding domain of (a) can be a synthetic polypeptide that binds the Fc portion of an immunoglobulin. Examples include, but are not limited to, a Kunitz peptide, a SMIP, an avimer, an affibody, a DARPin, or an anticalin.

In some embodiments, the chimeric receptor polypeptide disclosed herein can be a CAR polypeptide comprising an extracellular antigen binding domain (a). In some embodiments, the extracellular antigen binding domain of (a) is a single chain antibody fragment that binds to a tumor antigen, a pathogenic antigen, or an immune cell specific to an autoantigen. In certain embodiments, the tumor antigen is associated with a hematologic tumor. Examples include, but are not limited to, CD19, CD20, CD22, Kappa-chain, CD30, CD123, CD33, LeY, CD138, CD5, BCMA, CD7, CD40, and IL-1RAP. In certain embodiments, the tumor antigen is associated with a solid tumor. Examples include, but are not limited to, GD2, GPC3, FOLR (e.g., FOLR1 or FOLR2), HER2, EphA2, EFGRVIII, IL3RA2, VEGFR2, ROR1, NKG2D, EpCAM, CEA, Mesothelin, MUC1, CLDN18.2, CD171, CD133, PSCA, cMET, EGFR, PSMA, FAP, CD70, MUC16, L1-CAM, B7H3, and CAIX. In certain embodiments, the pathogenic antigen is a bacterial antigen, a viral antigen, or a fungal antigen, for example, those described herein. In some embodiments, the cytoplasmic signaling domain of (c) in any of the chimeric receptor polypeptides described herein (e.g., ACTR or CAR polypeptides) can be a cytoplasmic domain of CD3z or FcεR1γ.

In some embodiments, the hinge domain of any of the chimeric polypeptides described herein (e.g., ACTR or CAR polypeptides), when applicable, can be of CD28, CD16A, CD8a, IgG, murine CD8α or DAP12. In other examples, the hinge domain is a non-naturally occurring peptide. For example, the non-naturally occurring peptide may be an extended recombinant polypeptide (XTEN) or a (Gly₄Ser)ₙ polypeptide, in which n is an integer of 3-12, inclusive. In some examples, the hinge domain is a short segment, which may contain up to 60 amino acid residues.

In specific examples, an ACTR polypeptide as described herein may comprise (i) a CD28 co-stimulatory domain; and (ii) a CD28 transmembrane domain, a CD28 hinge domain, or a combination thereof. For example, the ACTR polypeptide comprises components (a)-(e) as shown in Table 4.

In specific examples, a CAR polypeptide described herein may comprise (i) a CD28 co-stimulatory domain or a 4-1BB co-stimulatory domain; and (ii) a CD28 transmembrane domain, a CD28 hinge domain, or a combination thereof. In further specific examples, a CAR polypeptide described herein may comprise (i) a CD28 co-stimulatory domain or a 4-1BB co-stimulatory domain, (ii) a CD8α transmembrane domain, a CD8α hinge domain, or a combination thereof.

The genetically engineered immune cells described herein, expressing the factor (e.g., polypeptide or nucleic acid) that redirects glucose metabolites and optionally the chimeric receptor polypeptide, may be a cell line or hematopoietic stem cell or a progeny thereof. In some embodiments, the genetically engineered immune cells can be natural killer (NK) cell, monocyte/macrophage, neutrophil, eosinophil, αβ T or γδ T cell.

In some examples, the immune cell is a T cell, in which the expression of an endogenous T cell receptor, an endogenous major histocompatibility complex, an endogenous beta-2-microglobulin, or a combination thereof has been inhibited or eliminated. In specific embodiments, the genetically engineered immune cells (e.g., NK, αβ T or γδ T cell) described herein may comprise a nucleic acid or a nucleic acid set, which collectively comprises: (a) a first nucleotide sequence encoding the factor (e.g., polypeptide or nucleic acid) that redirects glucose metabolites; and optionally (b) a second nucleotide sequence encoding the chimeric antigen receptor (CAR) polypeptide. The nucleic acid or the nucleic acid set is a DNA or RNA molecule or a set of DNA or RNA molecules. In some embodiments, the immune cell comprises the nucleic acid, which comprises both the first nucleotide sequence and the second nucleotide sequence. In some embodiments, the coding sequence of the factor (e.g., polypeptide or nucleic acid) that redirects glucose metabolites is upstream of that of the CAR polypeptide. In some embodiments, the coding sequence of the CAR polypeptide is upstream of that of the factor that redirects glucose metabolites. In some embodiments, the genetically engineered immune cells further may further comprise a third nucleotide sequence located between the first nucleotide sequence and the second nucleotide sequence, wherein the third nucleotide sequence encodes a ribosomal skipping site (e.g., a P2A peptide), an internal ribosome entry site (IRES), or a second promoter.

In some embodiments, the nucleic acid or the nucleic acid set is comprised within a vector or a set of vectors, which can be an expression vector or a set of expression vectors (e.g., viral vectors such as lentiviral vectors or retroviral vectors). A nucleic acid set or a vector set refers to a group of two or more nucleic acid molecules or two or more vectors, each encoding one of the polypeptides of interest (i.e., a polypeptide or nucleic acid that redirect glucose metabolites out of the glycolysis pathway and the CAR polypeptide). Any of the nucleic acids described herein is also within the scope of the present disclosure.

In another aspect, the present disclosure provides a pharmaceutical composition, comprising any of the immune cells described herein and a pharmaceutically acceptable carrier.

Moreover, provided herein is a method for inhibiting cells expressing a target antigen (e.g., reducing the number of such cells, blocking cell proliferation, and/or suppressing cell activity) in a subject, the method comprising administering to a subject in need thereof a population of the immune cells described herein, which may co-express the factor (e.g., polypeptide or nucleic acid) that redirects glucose metabolites and the CAR polypeptide. The subject (e.g., a human patient such as a human patient suffering from a cancer) may have been treated or is being treated with an anti-cancer therapy (e.g., an anti-cancer agent). In some examples, at least some of the cells expressing the target antigen are located in a low-glucose environment, a low-amino acid (e.g., low glutamine) environment, a low-pH environment, and/or a hypoxic environment, for example a tumor microenvironment.

In some examples, the immune cells are autologous. In other examples, the immune cells are allogeneic. In any of the methods described herein, the immune cells can be activated, expanded, or both ex vivo. In some instances, the immune cells comprise T cells, which are activated in the presence of one or more of anti-CD3 antibody, anti-CD28 antibody, IL-2, phytohemoagglutinin, and an engineered artificial stimulatory cell or particle. In other instances, the immune cells comprise natural killer cells, which are activated in the presence of one or more of 4-1BB ligand, anti-4-1BB antibody, IL-15, anti-IL-15 receptor antibody, IL-2, IL-12, IL-21 and K562 cells, an engineered artificial stimulatory cell or particle.

In some embodiments, the subject to be treated by the methods described herein may be a human patient suffering from a cancer, for example, carcinoma, lymphoma, sarcoma blastoma, and leukemia. Additional exemplary target cancer includes, but are not limited to, a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, liver cancer, and thyroid cancer. Exemplary cancers of B-cell origin is selected from the group consisting of B-lineage acute lymphoblastic leukemia, B-cell chronic lymphocytic leukemia, and B-cell non-Hodgkin's lymphoma.

The present disclosure also provides a nuclei acid or nucleic acid set, which collectively comprises: (A) a first nucleotide sequence encoding the factor that diverts or redirects glucose metabolites; and (B) a second nucleotide sequence encoding the chimeric receptor polypeptide (e.g., an ACTR, a CAR or a TCR polypeptide). An exemplary embodiment is a method for generating modified immune cells in vivo, the method comprising administering to a subject in need thereof the nucleic acid or nucleic acid set described herein, which may co-express the factor (e.g., polypeptide or nucleic acid) that redirects glucose metabolites and the CAR polypeptide.

Also, within the scope of the present disclosure are uses of the genetically engineered immune cells described herein, which co-express a factor (e.g., a polypeptide or a nucleic acid) that redirects glucose metabolites out of the glycolysis pathway and a CAR polypeptide for treating a target disease or disorder such as cancer or an infectious disorder and uses thereof for manufacturing a medicament for the intended medical treatment.

The details of one or more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a schematic illustration of exemplary polypeptides (open boxes) that re-direct glucose metabolites out of the glycolysis pathway. TIGAR reduces the activity of PFK. PKM2 and its loss of function (LoF) variants are less active than PKM1. In addition, GFPT1, competes with glycolytic enzymes for substrates and their products are re-directed into biosynthetic pathways. Glycolytic enzymes (solid boxes) belonging to the glycolysis pathway are ordered with their substrates and products.

FIG. 2 is a chart showing the impact of low glucose concentrations on proliferation of immune cells expressing a chimeric antigen receptor.

FIG. 3. is a chart depicting increased glucose uptake by T cells transduced with GLUT1, GOT2 and TIGAR relative to a mock transduced control (null) as a measure of fold change in luminescence.

FIG. 4. is a chart depicting free lactate produced by T cells transduced with GLUT1, GOT2 and TIGAR relative to mock transduced control (null) in the presence or absence of stimulation with PMA and ionomycin as a measure of fold change in luminescence.

FIG. 5. is an immunoblot showing transgene expression upon retroviral transduction with CAR only or CAR and transgene (GOT2 and TIGAR) relative to mock transduced control (null) in NK92 cells.

FIGS. 6A and 6B. are flow cytometric plots depicting CAR expression upon retroviral transduction with CAR only or CAR (FIG. 6A) and transgene (GOT2 and TIGAR) (FIG. 6B) relative to mock transduced control (null) in NK92 cells.

### DETAILED DESCRIPTION OF DISCLOSURE

Immune cell therapy involving genetically engineered T cells has shown promising effects in cancer therapy.

In some embodiments, the present disclosure provides expressing a chimeric receptor having an antigen-binding domain (e.g., CAR) fused to one or more T cell activation signaling domains. Binding of a cancer antigen via the antigen-binding domain results in T cell activation and triggers cytotoxicity. Recent results of clinical trials with infusions of chimeric receptor-expressing autologous T lymphocytes provided compelling evidence of their clinical potential. (Brentjens, Latouche et al., Nat Med, 9(3): 279-286 (2003); Pule et al., Nat Med, 14(11): 1264-1270 (2008); Brentjens et al., Blood, 118(18): 4817-4828 (2011); Porter et al., New England Journal of Medicine, 365(8): 725-733 (2011); Kochenderfer et al., Blood, 119(12): 2709-2720 (2012); Till et al., Blood, 119(17): 3940-3950 (2012); Brentjens et al., Sci Transl Med, 5(177): 177ra138 (2013)). Initially, the field was focusing on αβ T cells. More recently, cell-based therapy has expanded to include natural killer (NK) cells due to their unique advantages such as low risk of on-target/off-tumor toxicity in normal tissues, cytokine release syndrome and neurotoxicity. They also exhibit natural cytotoxicity against tumor cells. Finally, due to reduced graft versus host disease (GVHD), an off-the-shelf cell therapy product may be prepared (Schmidt et al., Front Immunol, 11: 611163 (2020); Wang et al., Cancer Lett, 472: 175-180 (2020); Xie et al., EBioMedicine, 59: 102975 (2020); Gong et al., J Hematol Oncol, 14(1): 73 (2021); Wrona et al., Int J Mol Sci, 22(11): (2021)). Another subtype of T cells, i.e., γδ T cells, have immense potential in cell therapy sharing similar advantages of that of NK cells additionally displaying immune regulatory functions (Sievers et al., Int J Mol Sci, 21(10): (2020); Park and Lee, Exp Mol Med, 53(3): 318-327 (2021)). In addition, persistent activation of these as T cells due to antigen dependent or independent CAR activation can lead to exhaustion and reduced bioactivity which is again an advantage of the innate cells such as NK and γδ T cells. Finally, both NK and γδ T cells expressing a chimeric receptor polypeptide may have increased effector functions such as increased inflammatory cytokine production, antigen acquisition and presentation or ability to activate adaptive immune responses.

In other embodiments, provided herein is to express an antibody-coupled T cell Receptor (ACTR) protein in a hematopoietic cell (e.g., a hematopoietic stem cell, an immune cell, such as an NK cell or a T cell), the ACTR protein containing an extracellular Fc-binding domain. When the ACTR-expressing hematopoietic cells (e.g., ACTR-expressing T cells, also called “ACTR T cells”) are administered to a subject together with an anti-cancer antibody, they may enhance toxicity against cancer cells targeted by the antibody via their binding to the Fc domain of the antibody (Kudo et al., Cancer Res, 74(1): 93-103 (2014)).

In yet other embodiments, provided herein is to express an exogenous T cell receptor (TCR) protein in an hematopoietic cell (e.g., a hematopoietic stem cell, an immune cell, such as an NK cell or a T cell or NKT cell), the TCR protein comprising an extracellular domain, or portion thereof, of a TCR α chain, a TCR β chain, a TCR γ chain, a TCR δ chain, a CD3 ε TCR subunit, a CD3 γ TCR subunit, a CD3ζ TCR subunit or a CD3 δ TCR subunit. In some instances, the TCR is further modified (e.g., express multiple TCRs against same or different target antigens or same or different epitopes on the same antigen or increasing affinity for the target or amino acid substitutions to increase preferential TCR chain pairing). When the TCR-expressing hematopoietic cells (e.g., TCR-expressing T cells, also called “TCR T cells”) are administered to a subject, they may enhance toxicity against cancer cells targeted by the TCR-CD3 complex via their binding to the peptide-MHC complex (Shafer et al., Front Immunol, 13: 835762 (2022); Wieczorek et al., Front Immunol, 8: 292 (2017)).

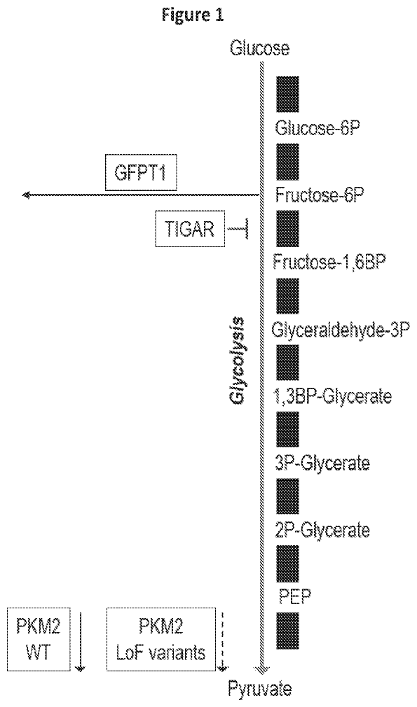

Tumor microenvironments have specific characteristics, such as low glucose, low amino acid, low pH, and/or hypoxic conditions, some of which may constrain the activity of effector immune cells such as effector T cells. The present disclosure is based, at least in part, on the development of strategies for enhancing effector immune cell activities in tumor microenvironments. In particular, the present disclosure features methods for enhancing the metabolic activity of the effector immune cells via diverting or re-directing glucose metabolism (e.g., diverting or re-directing one or more glucose metabolites out of the glycolysis pathway) in the effector immune cells, thereby enhancing their growth and bioactivity. Glucose metabolites refer to any molecule involved in glucose metabolism, including substrates used in any reaction in glucose metabolism and/or any product generated in such a reaction. Re-direction of glucose metabolites can be modulated by various factors, including the expression level of polypeptides that re-direct substrates metabolism or that decrease the rate of glucose breakdown by the glycolysis pathway. The present disclosure provides various approaches to redirect glucose metabolites out of the glycolysis pathway. Some examples are illustrated in FIG. 1, including: overexpressing an enzyme that competes with the enzymes of the glycolysis pathway for substrates and thereby re-directing those substrates into other biosynthetic pathways (e.g., GFPT1), overexpressing an enzyme that competes with, and is less active than, an enzyme of the glycolysis pathway for a substrate (e.g., PKM2, PKM2 variants), and/or overexpressing a polypeptide that reduces the function of an enzyme in the glycolysis pathway (e.g., TIGAR). Alternatively, the redirection of glucose metabolites can be increased by regulating expression of endogenous genes coding for proteins involved in redirecting glucose metabolism and/or regulating the cellular trafficking or activity of such proteins.

Accordingly, the present disclosure provides genetically engineered immune cells (e.g., NK, αβ T or γδ T or NKT cells) that possess altered glucose metabolism as compared with a native immune cell of the same type. Such genetically engineered immune cells express or overly express a polypeptide that diverts or redirect glucose metabolites out of the glycolysis pathway in the immune cells and express a chimeric receptor polypeptide comprising an extracellular target binding domain, a transmembrane domain and a cytoplasmic signaling domain, e.g., an antibody-coupled T cell receptor (ACTR) polypeptide, chimeric antigen receptor (CAR) or a T Cell Receptor (TCR) polypeptide. In a preferred embodiment, such polypeptide is overly expressed compared to a native immune cell of the same type, e.g., by factors (e.g., polypeptides) encoded by a transgene introduced into the immune cells (e.g., exogenous to the host cells). Also provided herein are uses of the genetically engineered immune cells, optionally in combination with an Fc-containing agent when needed (e.g., when the immune cells express an ACTR polypeptide), for improving immune cell proliferation, and/or an inhibiting or decreasing in target cells (e.g., target cancer cells) in a subject (e.g., a human cancer patient), e.g., via ADCC. The present disclosure also provides pharmaceutical compositions and kits comprising the described genetically engineered immune cells.

The genetically engineered immune cells described herein, expressing (e.g., over-expressing) a factor that redirects glucose metabolites out of the glycolysis pathway, may confer at least the following advantages. The expression of the factor that redirects glucose metabolism would re-direct glucose metabolites in the immune cells relative to native immune cells of the same type. As such, the genetically engineered immune cells may proliferate better, produce more cytokines, exhibit greater anti-tumor cytotoxicity, and/or exhibit greater survival of the respective NK, NKT, αβ T or γδ T cells in a low-glucose, low amino acid, low pH, and/or hypoxic environment (e.g., a tumor microenvironment) relative to immune cells that do not express (or do not over-express) the factor that redirects glucose metabolites, leading to enhanced cytokine production, survival rate, cytotoxicity, and/or anti-tumor activity.

I. Factors that Redirect Glucose Metabolites Out of Glycolysis Pathway

As used herein, a factor that redirects glucose metabolites refers to any factor (e.g., polypeptide, protein or nucleic acid) that redirects glucose out of the glycolysis pathway such that the glucose can be used in other biological pathways. For example, a factor may redirect glucose metabolites from the glycolysis pathway to pathways of: alcohol metabolism; carbohydrate and sugar metabolism; lipid and fatty acid metabolism; hormone metabolism; protein and amino acid metabolism; steroid metabolism; and/or vitamin and coenzyme metabolism. Said biological pathways include, but are not limited to, glycine, serine and threonine metabolism; alanine, aspartate and glutamine metabolism; lysine biosynthesis; arginine and proline metabolism; the pentose phosphate pathway; galactose metabolism; fructose and mannose metabolism; propanoate metabolism; butanoate metabolism; glyoxylate and dicarboxylate metabolism; the citrate cycle (TCA); amino sugar and nucleotide sugar metabolisms; starch and sucrose metabolisms; methane metabolism; oxidative phosphorylation; fatty acid metabolism; glutathione metabolism; 2-Oxocarboxylic acid metabolism; glycosylphosphatidylinositol (GPi)-anchor biosynthesis; N-glycan biosynthesis; pantothenate and CoA biosynthesis; terpenoid backbone biosynthesis; pyrimidine metabolism; and/or purine metabolism. Such a factor may re-direct glucose via any mechanism out of the glycolysis pathway.

In some instances, a factor that redirects glucose metabolites can be a polypeptide. As exemplified in FIG. 1, a factor that redirects glucose metabolites may be a polypeptide that: (i) competes with an enzyme of the glycolysis pathway for substrates (for example, GFPT1), thereby re-directing those substrates into other biosynthetic pathways (e.g., nucleotides via the pentose phosphate pathway, amino acids via the serine synthesis pathway, fatty acids via the glycerol-3P shuttle, or glycosylated proteins via the hexosamine synthesis pathway); (ii) competes with, and is less active than, an enzyme of the glycolysis pathway for a substrate (for example, PKM2); and/or (iii) reduces the function of an enzyme in the glycolysis pathway (for example, TIGAR).

In some embodiments, the polypeptide that diverts or redirects glucose metabolites is genetically engineered. In some embodiments, the polypeptide may be mutated to mimic an activated polypeptide that redirects glucose metabolites (e.g., a phosphorylation mimic) or mutated to impact its intracellular trafficking (e.g., traffic to the cell surface) such that the activity of the polypeptide is increased or decreased.

Any such polypeptide, which may be of any suitable species (e.g., mammalian such as human), may be contemplated for use with the compositions and methods described herein. Accordingly, in one example, the polypeptide that diverts or redirects glucose metabolites is PKM2. In another example, the polypeptide that diverts or redirects glucose metabolites is GFPT1. In yet another example, the polypeptide that diverts or redirects glucose metabolites is TIGAR.

GFPT1 is the first enzyme in the hexosamine pathway and controls the importation of glucose into the hexosamine pathway, i.e., re-direction of glucose metabolism. Specifically, GFPT1 catalyzes a reaction between L-glutamine and D-fructose 6-phosphate (Fructose-6P) to generate L-glutamate and D-glucosamine 6-phosphate. Additionally, GFPT1 is involved in the regulation of precursors for N- and O-linked glycosylation of proteins. Fructose-6P is a shared substrate between GFPT1 and PFK, an enzyme in the glycolysis pathway. Thus, enzymatic activity by GFPT1 to deplete Fructose-6P effectively re-directs glucose metabolites out of the glycolysis pathway and instead into the hexosamine and protein glycosylation pathways. Elevated GFPT1 expression or activity levels increase the re-direction of glucose metabolites away from the glycolysis pathway. The amino acid sequence of an exemplary human GFPT1 enzyme is provided below (SEQ ID NO: 68).

TIGAR functions to block glycolysis and re-direct glucose metabolites into the pentose phosphate shunt pathway. TIGAR is in direct opposition with PFKFB3 with respect to their shared regulation of fructose-2,6-bisphosphate, a molecule that increases the activity of the glycolytic pathway enzyme PFK. TIGAR degrades fructose-2,6-bisphosphate which effectively slows the enzymatic rate of PFK. This allows for more glucose metabolites to be re-directed into nucleotide synthesis and glycosylation pathways, e.g., the pentose phosphate shunt pathway. Elevated TIGAR expression or activity levels increase the re-direction of glucose metabolites away from the glycolysis pathway. The amino acid sequence of an exemplary human TIGAR enzyme is provided below (SEQ ID NO: 69).

In a specific embodiment, the polypeptide that diverts or redirects glucose metabolites out of the glycolysis pathway used in any of the modified hematopoietic cells such as immune cells is TIGAR. more specifically human TIGAR (SEQ ID NO: 69). Lactate production measured as a luminescence read-out of free L-Lactate directly correlates with glucose metabolism via glycolysis complimenting the glucose uptake rate measured as a luminescence read-out of free 2-deoxyglucose-6-phosphate (2DG6P). It was reported herein that T cells co-expressing TIGAR showed superior glucose uptake and lactate production as compared with co-expressed GLUT1 (see WO2020/010110, the relevant disclosures of which are incorporated by reference for the subject matter and purpose referenced herein) or GOT2 (see WO2020/037066, the relevant disclosures of which are incorporated by reference for the subject matter and purpose referenced herein), and such cells show improved survival rate under low glucose conditions found in the tumour microenvironment. Accordingly, therapeutic NK, αβ T cells or γδ T cells co-expressing a chimeric receptor polypeptide as disclosed herein (such as CAR or ACTR polypeptide) and TIGAR would be better adapted to the tumor microenvironment (for e.g., deficient in nutrients) and exhibit better therapeutic activity as compared with counterpart NK, αβ T or γδ T cell cells that do not co-express an exogenous TIGAR gene.

The term “TIGAR” encompasses functional equivalents of TIGAR (SEQ ID NO: 69) showing the same or substantially the same level of glucose uptake and/or lactate production when expressed in T cells, as shown in example 10. Substantially the same level means, in the context of an enzymatic activity or functional assay, the respective readout of the assay±25%, preferably ±20%, more preferably ±10%.

PKM2 is an isozyme of pyruvate kinase, a family of enzymes that convert phosphoenolpyruvate (PEP) to pyruvate, which is the final step in the glycolysis pathway. Compared to the other pyruvate kinase isozymes (e.g., PKM1), PKM2 is catalytically slow. Therefore, increasing the relative amount of PKM2 compared to the other isozymes increases the availability of PEP to other biosynthetic pathways (e.g., NFAT pathway and IL-2 production) and re-directs glucose metabolites. Elevated PSAT1 expression or activity levels increase the re-direction of glucose metabolites away from the glycolysis pathway. The amino acid sequence of an exemplary PKM2 enzyme is provided below (SEQ ID NO: 70). The polypeptide that redirects glucose metabolites may be a naturally-occurring polypeptide from a suitable species, for example, a mammalian polypeptide such as those derived from human or a non-human primate. Such naturally-occurring polypeptides are known in the art and can be obtained, for example, using any of the above-noted amino acid sequences as a query to search a publicly available gene database, for example GenBank. The polypeptide that redirects glucose metabolites for use in the instant disclosure may share a sequence identity of at least 85% (e.g., 90%, 95%, 97%, 98%, 99%, or above) as any of the exemplary proteins noted above.

The “percent identity” of two amino acid sequences is determined using the algorithm of (Karlin and Altschul, Proc Natl Acad Sci USA, 87(6): 2264-2268 (1990)), modified as in (Karlin and Altschul, Proc Natl Acad Sci USA, 90(12): 5873-5877 (1993)). Such an algorithm is incorporated into the NBLAST and XBLAST programs (version 2.0) of (Altschul et al., J Mol Biol, 215(3): 403-410 (1990)). BLAST protein searches can be performed with the XBLAST program, score=50, wordlength=3 to obtain amino acid sequences homologous to the protein molecules of the invention. Where gaps exist between two sequences, Gapped BLAST can be utilized as described in (Altschul et al., Nucleic Acids Res, 25(17): 3389-3402 (1997)). When utilizing BLAST and Gapped BLAST programs, the default parameters of the respective programs (e.g., XBLAST and NBLAST) can be used.

Alternatively, the polypeptide that redirects glucose metabolites may be a functional variant of a native counterpart. Such a functional variant may contain one or more mutations outside the functional domain(s) of the native counterpart. Functional domains of a native polypeptide that redirects glucose metabolites may be known in the art or can be predicted based on its amino acid sequence. Mutations outside the functional domain(s) would not be expected to substantially affect the biological activity of the protein. In some instances, the functional variant may exhibit an increased activity in glucose uptake as relative to the native counterpart. Alternatively, the functional variant may exhibit a decreased activity in glucose uptake as relative to the native counterpart. Additionally, the functional variant may have increased trafficking to the cell surface. Alternatively, the functional variant may have decreased trafficking to the cell surface.

For example, in some embodiments a polypeptide that redirects glucose metabolites pathway may be a functional variant of PKM2. Mutants that inhibit PKM2 function, e.g. through inhibition of binding to fructose 1,6-bisphosphate or through inhibition of the catalytic structure of PKM2, have been previously described, e.g., Y105E, Y105D, K422R, and H391Y (see, e.g., (Gupta et al., J Biol Chem, 285(22): 16864-16873 (2010); Iqbal et al., J Biol Chem, 289(12): 8098-8105 (2014); Wang et al., Protein Cell, 6(4): 275-287 (2015); Zhou et al., Cancer Res, 78(9): 2248-2261 (2018)). These variants are expected to have reduced activity relative to PKM2, and as such can be used as a way to modulate the activity or an overexpressed PKM2 polypeptide. In some embodiments, the PKM2 functional variant comprises at least one mutation selected from the list consisting of Y105E, Y105D, K422R, and H391Y. The amino acid sequences of exemplary human PKM2 variant enzymes are provided below: PKM2 Y105E is SEQ ID NO: 71, PKM2 Y105D is SEQ ID NO: 72, PKM2 K422R is SEQ ID NO: 73, PKM2 H391Y is SEQ ID NO: 74.

Alternatively or in addition, the functional variant may contain a conservative mutation(s) at one or more positions in the native counterpart (e.g., up to 20 positions, up to 15 positions, up to 10 positions, up to 5, 4, 3, 2, 1 position(s)). As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the factor that redirects glucose metabolites may be a molecule that regulates expression of an endogenous polypeptide that redirects glucose metabolites out of the glycolysis pathway. Such a factor may be a transcription factor or a microRNA. In some instances, the factor that redirects glucose metabolites can be a nucleic acid (e.g., DNA, microRNA, interfering RNA such as siRNA or shRNA, or antisense nucleic acid) that regulates expression of one or more enzymes involved in glucose metabolism (e.g., enzymes in the glycolysis pathway). In further embodiments, the factor that redirects glucose metabolites may be a transcription factor that regulates expression of one or more enzymes involved in glucose metabolism. In other embodiments, the factor that redirects glucose metabolites may be a molecule that mediates degradation of an endogenous factor such as those disclosed herein, for example an E3 ligase that is part of the ubiquitin/proteasome pathway. Additionally, the trafficking of a factor that redirects glucose metabolites may be modulated, for example, by expressing a polypeptide that increases its trafficking to a certain organelle or surface.

Table 1 below provides amino acid sequences of exemplary polypeptides that redirect glucose metabolites out of glycolysis pathway.

TABLE 1
Exemplary Polypeptides for Redirecting Glucose
Metabolites out of Glycolysis Pathway

| Polypeptides | Sequences | SEQ ID NO |
|---|---|---|
| GFPT1 | MCGIFAYLNYHVPRTRREILETLIKGLQRLEYRGYDS | SEQ ID NO. 68 |
| | AGVGFDGGNDKDWEANACKIQLIKKKGKVKALDEEVH | |
| | KQQDMDLDIEFDVHLGIAHTRWATHGEPSPVNSHPQR | |
| | SDKNNEFIVIHNGIITNYKDLKKFLESKGYDFESETD | |
| | TETIAKLVKYMYDNRESQDTSFTTLVERVIQQLEGAF | |
| | ALVFKSVHFPGQAVGTRRGSPLLIGVRSEHKLSTDHI | |
| | PILYRTARTQIGSKFTRWGSQGERGKDKKGSCNLSRV | |
| | DSTTCLFPVEEKAVEYYFASDASAVIEHTNRVIFLED | |
| | DDVAAVVDGRLSIHRIKRTAGDHPGRAVQTLQMELQQ | |
| | IMKGNFSSFMQKEIFEQPESVVNTMRGRVNFDDYTVN | |
| | LGGLKDHIKEIQRCRRLILIACGTSYHAGVATRQVLE | |
| | ELTELPVMVELASDFLDRNTPVFRDDVCFFLSQSGET | |
| | ADTLMGLRYCKERGALTVGITNTVGSSISRETDCGVH | |
| | INAGPEIGVASTKAYTSQFVSLVMFALMMCDDRISMQ | |
| | ERRKEIMLGLKRLPDLIKEVLSMDDEIQKLATELYHQ | |
| | KSVLIMGRGYHYATCLEGALKIKEITYMHSEGILAGE | |
| | LKHGPLALVDKLMPVIMIIMRDHTYAKCQNALQQVVA | |
| | RQGRPVVICDKEDTETIKNTKRTIKVPHSVDCLQGIL | |
| | SVIPLQLLAFHLAVLRGYDVDFPRNLAKSVTVE | |
| hTIGAR | MARFALTVVRHGETRFNKEKIIQGQGVDEPLSETGFK | SEQ ID NO. 69 |
| | QAAAAGIFLNNVKFTHAFSSDLMRTKQTMHGILERSK | |
| | FCKDMTVKYDSRLRERKYGVVEGKALSELRAMAKAAR | |
| | EECPVFTPPGGETLDQVKMRGIDFFEFLCQLILKEAD | |
| | QKEQFSQGSPSNCLETSLAEIFPLGKNHSSKVNSDSG | |
| | IPGLAASVLVVSHGAYMRSLFDYFLTDLKCSLPATLS | |
| | RSELMSVTPNTGMSLFIINFEEGREVKPTVQCICMNL | |
| | QDHLNGLTETR | |
| PKM2 | MSKPHSEAGTAFIQTQQLHAAMADTFLEHMCRLDIDS | SEQ ID NO. 70 |
| | PPITARNTGIICTIGPASRSVETLKEMIKSGMNVARL | |
| | NFSHGTHEYHAETIKNVRTATESFASDPILYRPVAVA | |
| | LDTKGPEIRTGLIKGSGTAEVELKKGATLKITLDNAY | |
| | MEKCDENILWLDYKNICKVVEVGSKIYVDDGLISLQV | |
| | KQKGADFLVTEVENGGSLGSKKGVNLPGAAVDLPAVS | |
| | EKDIQDLKFGVEQDVDMVFASFIRKASDVHEVRKVLG | |
| | EKGKNIKIISKIENHEGVRRFDEILEASDGIMVARGD | |
| | LGIEIPAEKVFLAQKMMIGRCNRAGKPVICATQMLES | |
| | MIKKPRPTRAEGSDVANAVLDGADCIMLSGETAKGDY | |
| | PLEAVRMOHLIAREAEAAIYHLQLFEELRRLAPITSD | |
| | PTEATAVGAVEASFKCCSGAIIVLTKSGRSAHQVARY | |
| | RPRAPIIAVTRNPQTARQAHLYRGIFPVLCKDPVQEA | |
| | WAEDVDLRVNFAMNVGKARGFFKKGDVVIVLTGWRPG | |
| | SGFTNTMRVVPVP | |
| PKM2 Y105E | MSKPHSEAGTAFIQTQQLHAAMADTFLEHMCRLDIDS | SEQ ID NO. 71 |
| | PPITARNTGIICTIGPASRSVETLKEMIKSGMNVARL | |
| | NFSHGTHEYHAETIKNVRTATESFASDPILERPVAVA | |
| | LDTKGPEIRTGLIKGSGTAEVELKKGATLKITLDNAY | |
| | MEKCDENILWLDYKNICKVVEVGSKIYVDDGLISLQV | |
| | KQKGADFLVTEVENGGSLGSKKGVNLPGAAVDLPAVS | |
| | EKDIQDLKFGVEQDVDMVFASFIRKASDVHEVRKVLG | |
| | EKGKNIKIISKIENHEGVRRFDEILEASDGIMVARGD | |
| | LGIEIPAEKVFLAQKMMIGRCNRAGKPVICATQMLES | |
| | MIKKPRPTRAEGSDVANAVLDGADCIMLSGETAKGDY | |
| | PLEAVRMQHLIAREAEAAIYHLQLFEELRRLAPITSD | |
| | PTEATAVGAVEASFKCCSGAIIVLTKSGRSAHQVARY | |
| | RPRAPIIAVTRNPQTARQAHLYRGIFPVLCKDPVQEA | |
| | WAEDVDLRVNFAMNVGKARGFFKKGDVVIVLTGWRPG | |
| | SGFTNTMRVVPVP | |
| PKM2 Y105D | MSKPHSEAGTAFIQTQQLHAAMADTFLEHMCRLDIDS | SEQ ID NO. 72 |
| | PPITARNTGIICTIGPASRSVETLKEMIKSGMNVARL | |
| | NFSHGTHEYHAETIKNVRTATESFASDPILDRPVAVA | |
| | LDTKGPEIRTGLIKGSGTAEVELKKGATLKITLDNAY | |
| | MEKCDENILWLDYKNICKVVEVGSKIYVDDGLISLQV | |
| | KQKGADFLVTEVENGGSLGSKKGVNLPGAAVDLPAVS | |
| | EKDIQDLKFGVEQDVDMVFASFIRKASDVHEVRKVLG | |
| | EKGKNIKIISKIENHEGVRRFDEILEASDGIMVARGD | |
| | LGIEIPAEKVFLAQKMMIGRCNRAGKPVICATQMLES | |
| | MIKKPRPTRAEGSDVANAVLDGADCIMLSGETAKGDY | |
| | PLEAVRMQHLIAREAEAAIYHLQLFEELRRLAPITSD | |
| | PTEATAVGAVEASFKCCSGAIIVLTKSGRSAHQVARY | |
| | RPRAPIIAVTRNPQTARQAHLYRGIFPVLCKDPVQEA | |
| | WAEDVDLRVNFAMNVGKARGFFKKGDVVIVLTGWRPG | |
| | SGFTNTMRVVPVP | |
| PKM2 K422R | MSKPHSEAGTAFIQTQQLHAAMADTFLEHMCRLDIDS | SEQ ID NO. 73 |
| | PPITARNTGIICTIGPASRSVETLKEMIKSGMNVARL | |
| | NFSHGTHEYHAETIKNVRTATESFASDPILYRPVAVA | |
| | LDTKGPEIRTGLIKGSGTAEVELKKGATLKITLDNAY | |
| | MEKCDENILWLDYKNICKVVEVGSKIYVDDGLISLQV | |
| | KQKGADFLVTEVENGGSLGSKKGVNLPGAAVDLPAVS | |
| | EKDIQDLKFGVEQDVDMVFASFIRKASDVHEVRKVLG | |
| | EKGKNIKIISKIENHEGVRRFDEILEASDGIMVARGD | |
| | LGIEIPAEKVFLAQKMMIGRCNRAGKPVICATQMLES | |
| | MIKKPRPTRAEGSDVANAVLDGADCIMLSGETAKGDY | |
| | PLEAVRMQHLIAREAEAAIYHLQLFEELRRLAPITSD | |
| | PTEATAVGAVEASFRCCSGAIIVLTKSGRSAHQVARY | |
| | RPRAPIIAVTRNPQTARQAHLYRGIFPVLCKDPVQEA | |
| | WAEDVDLRVNFAMNVGKARGFFKKGDVVIVLTGWRPG | |
| | SGFTNTMRVVPVP | |
| PKM2 H391Y | MSKPHSEAGTAFIQTQQLHAAMADTFLEHMCRLDIDS | SEQ ID NO. 74 |
| | PPITARNTGIICTIGPASRSVETLKEMIKSGMNVARL | |
| | NFSHGTHEYHAETIKNVRTATESFASDPILYRPVAVA | |
| | LDTKGPEIRTGLIKGSGTAEVELKKGATLKITLDNAY | |
| | MEKCDENILWLDYKNICKVVEVGSKIYVDDGLISLQV | |
| | KQKGADFLVTEVENGGSLGSKKGVNLPGAAVDLPAVS | |
| | EKDIQDLKFGVEQDVDMVFASFIRKASDVHEVRKVLG | |
| | EKGKNIKIISKIENHEGVRRFDEILEASDGIMVARGD | |
| | LGIEIPAEKVFLAQKMMIGRCNRAGKPVICATQMLES | |
| | MIKKPRPTRAEGSDVANAVLDGADCIMLSGETAKGDY | |
| | PLEAVRMQHLIAREAEAAIYYLQLFEELRRLAPITSD | |
| | PTEATAVGAVEASFKCCSGAIIVLTKSGRSAHQVARY | |
| | RPRAPIIAVTRNPQTARQAHLYRGIFPVLCKDPVQEA | |
| | WAEDVDLRVNFAMNVGKARGFFKKGDVVIVLTGWRPG | |
| | SGFTNTMRVVPVP | |
| GLUT1 | MEPSSKKLTGRLMLAVGGAVLGSLQFGYNTGVINAPQ | SEQ ID NO. 80 |
| | KVIEEFYNQTWVHRYGESILPTTLTTLWSLSVAIFSV | |
| | GGMIGSFSVGLFVNRFGRRNSMLMMNLLAFVSAVLMG | |
| | FSKLGKSFEMLILGRFIIGVYCGLTTGFVPMYVGEVS | |
| | PTALRGALGTLHQLGIVVGILIAQVFGLDSIMGNKDL | |
| | WPLLLSIIFIPALLQCIVLPFCPESPRFLLINRNEEN | |
| | RAKSVLKKLRGTADVTHDLQEMKEESRQMMREKKVTI | |
| | LELFRSPAYRQPILIAVVLQLSQQLSGINAVFYYSTS | |
| | IFEKAGVQQPVYATIGSGIVNTAFTVVSLFVVERAGR | |
| | RTLHLIGLAGMAGCAILMTIALALLEQLPWMSYLSIV | |
| | AIFGFVAFFEVGPGPIPWFIVAELFSQGPRPAAIAVA | |
| | GFSNWTSNFIVGMCFQYVEQLCGPYVFIIFTVLLVLF | |
| | FIFTYFKVPETKGRTFDEIASGFRQGGASQSDKTPEE | |
| | LFHPLGADSQV | |
| GOT2 | MALLHSGRVLPGIAAAFHPGLAAAASARASSWWTHVE | SEQ ID NO. 81 |
| | MGPPDPILGVTEAFKRDTNSKKMNLGVGAYRDDNGKP | |
| | YVLPSVRKAEAQIAAKNLDKEYLPIGGLAEFCKASAE | |
| | LALGENSEVLKSGRFVTVQTISGTGALRIGASFLQRF | |
| | FKFSRDVFLPKPTWGNHTPIFRDAGMQLQGYRYYDPK | |
| | TCGFDFTGAVEDISKIPEQSVLLLHACAHNPTGVDPR | |
| | PEQWKEIATVVKKRNLFAFFDMAYQGFASGDGDKDAW | |
| | AVRHFIEQGINVCLCQSYAKNMGLYGERVGAFTMVCK | |
| | DADEAKRVESQLKILIRPMYSNPPLNGARIAAAILNT | |
| | PDLRKQWLQEVKVMADRIIGMRTQLVSNLKKEGSTHN | |
| | WQHITDQIGMFCFTGLKPEQVERLIKEFSIYMTKDGR | |
| | ISVAGVTSSNVGYLAHAIHQVTK | |

### II. Chimeric Receptor Polypeptides

As used herein, a chimeric receptor polypeptide refers to a non-naturally occurring molecule that can be expressed on the surface of a host cell, preferably of an immune cell. The extracellular target binding domain of the chimeric receptor polypeptide targets an antigen of interest (e.g., an antigen associated with a disease such as cancer or an antigen associated with a pathogen; see discussions herein). The extracellular target binding domain may bind to an antigen of interest directly (e.g., an extracellular antigen binding domain in a CAR polypeptide as disclosed herein). Alternatively, the extracellular target binding domain may bind to the antigen of interest via an intermediate, for example, an Fc-containing agent such as an antibody. Further, the extracellular target binding may occur by engagement between the TCR-CD3 complex specific to a peptide-MHC complex (e.g., via binding of a genetically engineered T cell expressing a TCR polypeptide as disclosed herein to an antigen-presenting cell such as tumor cells displaying the peptide-MHC complex). A chimeric receptor polypeptide may further comprise a hinge domain, one or more co-stimulatory domains, or a combination thereof. In some instances, the chimeric receptor polypeptide may be free of co-stimulatory domains. The chimeric receptor polypeptides are configured such that, when expressed in a host cell, the extracellular target binding domain is located extracellularly for binding to a target antigen, directly or indirectly. The optional co-stimulatory signaling domain may be located in the cytoplasm for triggering activation and/or effector signaling.

In some embodiments, the chimeric receptor polypeptide comprises one or more of the following features: (i) the chimeric receptor polypeptide further comprises at least one co-stimulatory signaling domain or is free of co-stimulatory signaling domains; (ii) the cytoplasmic signaling domain (c) comprises an immunoreceptor tyrosine-based activation motif (ITAM); (iii) the cytoplasmic signaling domain (c) is located at the C-terminus of the chimeric receptor polypeptide; (iv) the chimeric receptor polypeptide further comprises a hinge domain, which is located at the C-terminus of (a) and the N-terminus of (b), (v) the chimeric receptor polypeptide is free of any hinge domain; and (vi) the chimeric receptor polypeptide further comprises a signal peptide at its N-terminus.

In some embodiments, chimeric receptor polypeptides described herein may further comprise a hinge domain, which may be located at the C-terminus of the extracellular target binding domain and the N-terminus of the transmembrane domain. The hinge may be of any suitable length. In other embodiments, the chimeric receptor polypeptide described herein may have no hinge domain at all. In yet other embodiments, the chimeric receptor polypeptide described herein may have a shortened hinge domain (e.g., including up to 25 amino acid residues).

In some embodiments, a chimeric receptor polypeptide as described herein may comprise, from N-terminus to C-terminus, the extracellular target binding domain, the transmembrane domain, and the cytoplasmic signaling domain. In some embodiments, a chimeric receptor polypeptide as described herein comprises, from N-terminus to C-terminus, the extracellular target binding domain, the transmembrane domain, at least one co-stimulatory signaling domain, and the cytoplasmic signaling domain. In other embodiments, a chimeric receptor polypeptide as described herein comprises, from N-terminus to C-terminus, the extracellular target binding domain, the transmembrane domain, the cytoplasmic signaling domains, and at least one co-stimulatory signaling domain.

In some embodiments, the chimeric receptor polypeptide can be an antibody-coupled T cell receptor (ACTR) polypeptide. As used herein, an ACTR polypeptide (a.k.a., an ACTR construct) refers to a non-naturally occurring molecule that can be expressed on the surface of a host cell and comprises an extracellular domain with binding affinity and specificity for the Fc portion of an immunoglobulin (“Fc binder” or “Fc binding domain”), a transmembrane domain, and a cytoplasmic signaling domain. In some embodiments, the ACTR polypeptides described herein may further include at least one co-stimulatory signaling domain.

In other embodiments, the chimeric receptor polypeptide disclosed herein may be a chimeric antigen receptor (CAR) polypeptide. As used herein, a CAR polypeptide (a.k.a., a CAR construct) refers to a non-naturally occurring molecule that can be expressed on the surface of a host cell and comprises an extracellular antigen binding domain, a transmembrane domain, and a cytoplasmic signaling domain. The CAR polypeptides described herein may further include at least one co-stimulatory signaling domain.

The extracellular antigen binding domain may be any peptide or polypeptide that specifically binds to a target antigen, including naturally occurring antigens that are associated with a medical condition (e.g., a disease), or an antigenic moiety conjugated to a therapeutic agent that targets a disease-associated antigen.

In some embodiments, the CAR polypeptides described herein may further include at least one co-stimulatory signaling domain. The CAR polypeptides are configured such that, when expressed on a host cell, the extracellular antigen-binding domain is located extracellularly for binding to a target molecule and the cytoplasmic signaling domain. The optional co-stimulatory signaling domain may be located in the cytoplasm for triggering activation and/or effector signaling.

As used herein, the phrase “a protein X transmembrane domain” (e.g., a CD8 transmembrane domain) refers to any portion of a given protein, i.e., transmembrane-spanning protein X, that is thermodynamically stable in a membrane.

As used herein, the phrase “a protein X cytoplasmic signaling domain,” for example, a CD3z cytoplasmic signaling domain, refers to any portion of a protein (protein X) that interacts with the interior of a cell or organelle and is capable of relaying a primary signal as known in the art, which lead to immune cell proliferation and/or activation. The cytoplasmic signaling domain as described herein differs from a co-stimulatory signaling domain, which relays a secondary signal for fully activating immune cells.

As used herein, the phrase “a protein X co-stimulatory signaling domain,” e.g., a CD28 co-stimulatory signaling domain, refers to the portion of a given co-stimulatory protein (protein X, such as CD28, 4-1BB, OX40, CD27, or ICOS) that can transduce co-stimulatory signals (secondary signals) into immune cells (such as T cells), leading to fully activation of the immune cells.

In some embodiments the extracellular target binding domain, preferably the antigen binding domain, is a single chain variable fragment (scFv) or a single domain antibody that binds to a tumor antigen, a pathogenic antigen, or an immune cell specific to an autoantigen.

In some embodiments, the TCRs can include a modified T Cell Receptor (TCR). In some embodiments, a modified TCR may comprise a heterodimer comprising α and/or β chain, as well as one or more CD3 chains (e.g., γ, δ, ε, or ζ) chains, and optionally that are engineered to bind an antigen-specific peptide complexed with antigen presenting molecules such as MHC class I or MHC class II on a target cell (e.g., a tumor cell expressing WTl or NY-ESO-1, a pathogen-infected cell such as HPV16 E6 protein).

### A. Extracellular Target Binding Domain

The chimeric receptor polypeptides disclosed herein comprise an extracellular domain that targets an antigen of interest (e.g., those described herein) via either direct binding or indirectly binding (through an intermediate such as an antibody). The chimeric receptor polypeptides may be ACTR polypeptides that comprise an Fc binding domain. Alternatively, the chimeric receptor polypeptides may be CAR polypeptides that comprise an extracellular antigen binding domain.

### (i) Fc Binding Domains

The ACTR polypeptides described herein comprise an extracellular domain that is an Fc binding domain, i.e., capable of binding to the Fc portion of an immunoglobulin (e.g., IgG, IgA, IgM, or IgE) of a suitable mammal (e.g., human, mouse, rat, goat, sheep, or monkey). Suitable Fc binding domains may be derived from naturally occurring proteins such as mammalian Fc receptors or certain bacterial proteins (e.g., protein A, protein G). Additionally, Fc binding domains may be synthetic polypeptides engineered specifically to bind the Fc portion of any of the antibodies described herein with high affinity and specificity. For example, such an Fc binding domain can be an antibody or an antigen-binding fragment thereof that specifically binds the Fc portion of an immunoglobulin. Examples include, but are not limited to, a single-chain variable fragment (scFv), a domain antibody, or single domain antibodies (e.g., nanobodies). Alternatively, an Fc binding domain can be a synthetic peptide that specifically binds the Fc portion, such as a Kunitz domain, a small modular immunopharmaceutical (SMIP), an adnectin, an avimer, an affibody, a DARPin, or an anticalin, which may be identified by screening a peptide combinatory library for binding activities to Fc.

In some embodiments, the Fc binding domain is an extracellular ligand-binding domain of a mammalian Fc receptor. As used herein, an “Fc receptor” is a cell surface bound receptor that is expressed on the surface of many immune cells (including B cells, dendritic cells, natural killer (NK) cells, macrophage, neutrophils, mast cells, and eosinophils) and exhibits binding specificity to the Fc domain of an antibody. Fc receptors are typically comprised of at least two immunoglobulin (Ig)-like domains with binding specificity to an Fc (fragment crystallizable) portion of an antibody. In some instances, binding of an Fc receptor to an Fc portion of the antibody may trigger antibody dependent cell-mediated cytotoxicity (ADCC) effects. The Fc receptor used for constructing an ACTR polypeptide as described herein may be a naturally occurring polymorphism variant (e.g., the CD16 V158 variant), which may have increased or decreased affinity to Fc as compared to a wild-type counterpart. Alternatively, the Fc receptor may be a functional variant of a wild-type counterpart, which carry one or more mutations (e.g., up to 10 amino acid residue substitutions including 1, 2, 3, 4, 5, 6, 7, 8, 9, or 10 mutations) that alter the binding affinity to the Fc portion of an Ig molecule. In some instances, the mutation may alter the glycosylation pattern of the Fc receptor and thus the binding affinity to Fc.

Table 2 lists a number of exemplary polymorphisms in Fc receptor extracellular domains see, e.g., (Kim et al., Journal of Molecular Evolution, 53(1): 1-9 (2001)) which may be used in any of the methods or constructs described herein:

TABLE 2
Exemplary Polymorphisms in Fc Receptors

| | Amino Acid Number | | | | | | | | | |
|---|---|---|---|---|---|---|---|---|---|---|
| | 19 | 48 | 65 | 89 | 105 | 130 | 134 | 141 | 142 | 158 |
| FCR10 | R | S | D | I | D | G | F | Y | T | V |
| P08637 | R | S | D | I | D | G | F | Y | I | F |
| S76824 | R | S | D | I | D | G | F | Y | I | V |
| J04162 | R | N | D | V | D | D | F | H | I | V |
| M31936 | S | S | N | I | D | D | F | H | I | V |
| M24854 | S | S | N | I | E | D | S | H | I | V |
| X07934 | R | S | N | I | D | D | F | H | I | V |
| X14356 (FcgRII) | N | N | N | S | E | S | S | S | I | I |
| M31932 (FcgRI) | S | T | N | R | E | A | F | T | I | G |
| X06948 (FcaeI) | R | S | E | S | Q | S | E | S | I | V |

Fc receptors are classified based on the isotype of the antibody to which it is able to bind. For example, F-gamma receptors (FcR) generally bind to IgG antibodies, such as one or more subtype thereof (i.e., IgG1, IgG2, IgG3, IgG4); Fe-alpha receptors (FR) generally bind to IgA antibodies; and Fe-epsilon receptors (FcεR) generally bind to IgE antibodies. In some embodiments, the Fc receptor is an FcγR, an FcαR, or an FcεR. Examples of FcγR include, without limitation, CD64A, CD64B, CD64C, CD32A, CD32B, CD16A, and CD16B. An example of an FcαR is FcαR1/CD89. Examples of FcεR include, without limitation, FcεRI and FcεRII/CD23. Table 3 lists exemplary Fc receptors for use in constructing the ACTR polypeptides described herein and their binding activity to corresponding Fc domains:

TABLE 3
Exemplary Fc Receptors

| | Principal | |
|---|---|---|
| Receptor name | antibody ligand | Affinity for ligand |
| FcγRI (CD64) | IgG1 and IgG3 | High (Kd ~10⁻⁹ M) |
| FcγRIIA (CD32) | IgG | Low (Kd >10⁻⁷ M) |
| FcγRIIB1 (CD32) | IgG | Low (Kd >10⁻⁷ M) |
| FcγRIIB2 (CD32) | IgG | Low (Kd >10⁻⁷ M) |
| FcγRIIIA (CD16a) | IgG | Low (Kd >10⁻⁶ M) |
| FcγRIIIB (CD16b) | IgG | Low (Kd >10⁻⁶ M) |
| FcεRI | IgE | High (Kd ~10⁻¹⁰ M) |
| FcεRII (CD23) | IgE | Low (Kd >10⁻⁷ M) |
| FcαRI (CD89) | IgA | Low (Kd >10⁻⁶ M) |
| Fcα/μR | IgA and IgM | High for IgM, Mid for IgA |
| FcRn | IgG | |

Selection of the ligand binding domain of an Fc receptor for use in the ACTR polypeptides described herein will be apparent to one of skill in the art. For example, it may depend on factors such as the isotype of the antibody to which binding of the Fc receptor is desired and the desired affinity of the binding interaction.

In some examples, the Fc binding domain is the extracellular ligand-binding domain of CD16, which may incorporate a naturally occurring polymorphism that may modulate affinity for Fc. In some examples, the Fc binding domain is the extracellular ligand-binding domain of CD16 incorporating a polymorphism at position 158 (e.g., valine or phenylalanine). In some embodiments, the Fc binding domain is produced under conditions that alter its glycosylation state and its affinity for Fc.

The amino acid sequences of human CD16A F158 (SEQ ID NO: 75) and CD16A V158 (SEQ ID NO: 76) variants are provided herewith with the F158 and V158 residue in bold/underlined.

CD16A F158 (F158 bold/underlined)
(SEQ ID NO: 75)
MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLFGSKNVSSETVNITITQGLAVSTIS
SFFPPGYQVSFCLVMVLLFAVDTGLYFSVKTNIRSSTRDWKDHKFKWRKD
PQDK
CD16A V158 (V158 bold/underlined)
(SEQ ID NO: 76)
MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLVGSKNVSSETVNITITQGLAVSTIS
SFFPPGYQVSFCLVMVLLFAVDTGLYFSVKTNIRSSTRDWKDHKFKWRKD
PQDK

In some embodiments, the Fc binding domain is the extracellular ligand-binding domain of CD16 incorporating modifications that render the ACTR polypeptide specific for a subset of IgG antibodies. For example, mutations that increase or decrease the affinity for an IgG subtype (e.g., IgG1) may be incorporated.

Any of the Fc binding domains described herein may have a suitable binding affinity for the Fc portion of a therapeutic antibody. As used herein, “binding affinity” refers to the apparent association constant or K_{A}. The K_{A} is the reciprocal of the dissociation constant, K_{D}. The extracellular ligand-binding domain of an Fc receptor domain of the ACTR polypeptides described herein may have a binding affinity K_{d} of at least 10⁻⁵, 10⁻⁶, 10⁻⁷, 10⁻⁸, 10⁻⁹, 10⁻¹⁰ M or lower for the Fc portion of antibody. In some embodiments, the Fc binding domain has a high binding affinity for an antibody, isotype(s) of antibodies, or subtype(s) thereof, as compared to the binding affinity of the Fc binding domain to another antibody, isotype(s) of antibodies, or subtypes(s) thereof. In some embodiments, the extracellular ligand-binding domain of an Fc receptor has specificity for an antibody, isotype(s) of antibodies, or subtype(s) thereof, as compared to binding of the extracellular ligand-binding domain of an Fc receptor to another antibody, isotype(s) of antibodies, or subtypes(s) thereof.

Other Fc binding domains as known in the art may also be used in the ACTR constructs described herein including, for example, those described in WO2015/058018A1 and WO2018/140960, the relevant disclosures of each of which are incorporated by reference for the purpose and subject matter referenced herein.

### (ii) Extracellular Antigen Binding Domains

The CAR polypeptides described herein comprise an extracellular antigen binding domain, which re-directs the specificity of immune cells (e.g., NK, αβ T or γδ T cells) expressing the CAR polypeptide. As used herein, “an extracellular antigen binding domain” refers to a peptide or polypeptide having binding specificity to a target antigen of interest, which can be a naturally occurring antigen associated with a medical condition (e.g., expressed in a disease, condition or cell type). Non-limiting examples of the extracellular antigen binding domains are tumor antigens, pathogenic antigens and immune cells specific to an autoantigen (Gubin et al., J Clin Invest, 125(9): 3413-3421 (2015); Linnemann et al., Nat Med, 21(1): 81-85 (2015)). Respective diseases and/or conditions to be treated include tumors, inflammatory conditions and auto-immune disorders. In some embodiments, the antigen is selectively expressed or overexpressed on cells of the disease or condition, e.g., the tumor or pathogenic cells, as compared to normal or non-targeted cells or tissues.

In some embodiments the extracellular antigen binding domain of binds to a tumor antigen, which is associated with a hematologic or solid tumor.

Non-limiting examples of hematologic tumor extracellular binding domains are domains of CD19, CD20, CD22, Kappa-chain, CD30, CD123, CD33, LeY, CD138, CD5, BCMA, CD7, CD40, and IL-1RAP. Non-limiting examples of solid tumor extracellular binding domains are domains of GD2, GPC3, FOLR (e.g., FOLR1 or FOLR2), HER2, EphA2, EFGRVIII, IL13RA2, VEGFR2, ROR1, NKG2D, EpCAM, CEA, Mesothelin, MUC1, CLDN18.2, CD171, CD133, PSCA, cMET, EGFR, PSMA, FAP, CD70, MUC16, L1-CAM, B7H3, and CAIX. In other instances, Tumor antigens include tumor antigens derived from cancers that are characterized by tumor-associated antigen expression, such as HER2 expression. Antigens may include epitopic regions or epitopic peptides derived from genes mutated in tumor cells or from genes transcribed at different levels in tumor cells compared to normal cells (e.g., survivin, mutated Ras, bcr/abl rearrangement, HER2, mutated or wild-type p53).

Next, the antigenic moiety may be conjugated to a therapeutic agent that targets a disease-associated antigen. The extracellular antigen binding domain as described herein does not comprise an extracellular domain of an Fc receptor, and may not bind to the Fc portion of an immunoglobulin. An extracellular domain that does not bind to an Fc fragment means that the binding activity between the two is not detectable using a conventional assay or only background or biologically insignificant binding activity is detected using the conventional assay.

In some instances, the extracellular antigen binding domain of any CAR polypeptides described herein is a peptide or polypeptide capable of binding to a cell surface antigen (e.g., a native and mutated tumor antigen), or an antigen (or a fragment thereof) that is in a complex with a major histocompatibility complex and may be presented on the cell surface of an antigen-presenting cell. Such an extracellular antigen binding domain may be a single-chain antibody fragment (scFv), which may be derived from an antibody that binds the target cell surface antigen with a high binding affinity. Table 4 lists exemplary cell-surface target antigens and exemplary antibodies binding to such.

TABLE 4
Exemplary Cell Surface Target Antigen and Exemplary Antibodies Binding to such

| | | | Exemplary |
|---|---|---|---|
| Exemplary target | Exemplary | Exemplary target | antibodies and Fc- |
| antigens | antibodies | antigens | fusion agents |
| CD137 (4-1BB) | utomilumab | CD74 | milatuzumab |
| Trophoblast | naptumomab | HLA-DR | IMMU-114 |
| glycoprotein (5T4) | estafenatox | | |
| Adenosine A2a | anti-A2aR mAbs | Hsp70 | mi-TUMEXtx |
| receptor (A2aR) | | | |
| Alk-1 protein kinase | ascrinvacumab | Hsp 90 | ZSG-102 |
| (ACVRL1) | | | |
| ADAM-10 | 8C7 | ICAM-1 | BI-505 |
| (ADAM10) | | | |
| TACE (ADAM17) | MEDI-3622 | Inducible T-cell co- | GSK-3359609 |
| | | stimulator (ICOS) | |
| ADAM-28 | GFC-201 | Immunoglobulin kappa | KappaMab |
| (ADAM28) | | (Ig kappa) | |
| CD156; | MAB-1031 | Immunoglobulin antigen | LambdaMab |
| Immunoglobulin G1; | | (Ig lambda) | |
| Immunoglobulin G2 | | | |
| (ADAM8) | | | |
| ADAM-9 (ADAM9) | AEX-6003 | IL-6 receptor (IL-6R) | tocilizumab |
| Anterior gradient | agtuzumab | IL-7 receptor (IL-7R) | anti-IL7R mAbs |
| protein 2 homolog | | | |
| (AGR2) | | | |
| Anaplastic | KTN-0125 | IL-13 receptor alpha 1 | ASLAN-004 |
| lymphoma kinase | | subunit (IL13RA1) | |
| (ALK) | | | |
| Angiopoietin ligand- | vanucizumab | IL-13 receptor alpha 2 | anti-IL13RA2 |
| 2 (Ang-2); Vascular | | subunit (IL13RA2) | mAbs |
| endothelial growth | | | |
| factor-A (VEGF-A) | | | |
| Lactadherin (Anti- | TriAb (11D10) | IL-1 receptor accessory | CAN-04 |
| idiotype) | | protein (IL1RAP) | |
| Tumor necrosis | BION-1301 | IL-2 receptor beta (IL2R | Mikbeta1 |
| factor ligand 13 | | beta) | |
| (APRIL) | | | |
| Aspartate beta- | PAN-622 | Immunoglobulin like | BAY-1905254 |
| hydroxylase (ASPH) | | domain receptor 2 | |
| | | (ILDR2) | |
| Axl tyrosine kinase | BA-3011 | Integrin alpha-X/beta-1 | anti-Integrin a10b1 |
| (AXL) | | (Integrin a10b1) | mAbs |
| CD276 antigen (B7- | BVD m276; | Integrin alpha-3/beta-1 | BCMab-1 |
| H3) | hu8H9 | (Integrin a3b1) | |
| V-set domain- | FPA-150 | Integrin alpha-6/beta-4 | 90Y-ITGA6B4 |
| containing T-cell | | (Integrin a6b4) | |
| activation inhibitor 1 | | | |
| (VTCN1; also B7- | | | |
| H4) | | | |
| B-cell activating | blisibimod | Integrin alpha-9 (Integrin | GND-001 |
| factor; (BAFF; also | | a9) | |
| TNFSF13B and | | | |
| CD257) | | | |
| B-cell activating | VAY736 | CD49b (Integrin alpha 2) | Vatelizumab |
| factor receptor; | | | |
| (BAFF-R; also | | | |
| TNFSF13C and | | | |
| CD268) | | | |
| BAG molecular | anti-BAG3 | CD49c (Integrin alpha 3) | anti-CD49c mAbs |
| chaperone regulator | mAbs | | |
| 3 (BAG3) | | | |
| Basigin (BSG; | cHAb18 | CD49d; (Integrin alpha | anti-CD49d mAbs |
| CD147) | | 4) | |
| B-cell maturation | SEA-BCMA | CD51 | abituzumab |
| antigen (BCMA; | | | |
| also TNFRSF17) | | | |
| ADP ribosyl | OX-001 | CD29 (integrin beta 1) | OS-2966 |
| cyclase-2 (BST1) | | | |
| B and T lymphocyte | 40E4 | CD61 (Integrin beta 3) | anti-CD61 mAbs |
| attenuator (BTLA) | | | |
| Complement C5a | neutrazumab | Jagged-1 | anti-Jagged-1 |
| receptor (C5aR) | | | mAbs |
| CACNA2D1 | anti- | Kidney-associated | AB-3A4 |
| calcium channel | CACNA2D1 | antigen 1 (KAAG1) | |
| subunit | mAbs | | |
| (CACNA2D1) | | | |
| Carbonic anhydrase- | G250 | Potassium channel | Y-4 |
| IX (CAIX) | | subfamily K member 9 | |
| | | (KCNK9) | |
| Calreticulin (CALR) | Anti-CALR | KIR2DL1/2L3 | lirilumab |
| | mAbs | | |
| Caveolin 1 (CAV1) | anti-CAV1 | tyrosine-protein kinase | CDX-0158 |
| | mAbs | kit (KIT) | |
| Carbonic anhydrase- | 177Lu-6A10- | L1CAM | anti-L1CAM mAbs |
| XII (CAXII) | Fab; anti-CAXII | | |
| | mAbs | | |
| CCR2 chemokine | plozalizumab | Death receptor 5 (DR5) | APOMAB |
| receptor (CCR2) | | | |
| CCR3 chemokine | anti-CCR3 | CD223 (LAG3) | relatlimab |
| receptor (CCR3) | mAbs | | |
| CCR4 chemokine | mogamulizumab | Lewis Y | hu3S193; MB311 |
| receptor (CCR4) | | | |
| CCR5 chemokine | PRO 140; | Zinc transporter | SGN-LIV1 |
| receptor (CCR5) | CCR5mAb004 | SLC39A6 (LIV1) | |
| CCR7 chemokine | anti-CCR7 | Lysyl oxidase-like | AB-0023 |
| receptor (CCR7) | mAbs | protein 2 (LOXL2) | |
| CCR9 chemokine | anti-CCR9 | Leucine rich repeat- | ABBV-085 |
| receptor (CCR9) | mAbs | containing protein 15 | |
| | | (LRRC15) | |
| Interleukin-3 | CSL362; | Leucine rich repeat- | ARGX-115 |
| receptor alpha | KHK2823 | containing protein 32 | |
| (IL3RA; CD123) | | (LRRC32) | |
| Aminopeptidase N | MI-130110 | Lymphocyte antigen 75 | MEN-1309 |
| (CD13) | | (LY75) | |
| Prominin 1 (CD133) | anti-CD133 | Ly6/PLAUR domain- | BAY-1129980 |
| | mAbs | containing protein 3 | |
| | | (LYPD3) | |
| Syndecan-1 (CD138) | indatuximab | Melanoma associated | LxC-002 |
| | ravtansine | antigen (MAGE peptide | |
| | | presented in MHC) | |
| CD160 | ELB-021 | Matriptase (ST14) | anti-ST14 mAbs |
| Activated leukocyte | CX-2009 | MICA/B | IPH4301 |
| cell adhesion | | | |
| molecule (CD166) | | | |
| B-lymphocyte | MOR208 | MIF/HLA-A2 (MIF | RL21A |
| antigen CD19 | | peptide presented in | |
| | | MHC) | |
| B-lymphocyte | rituximab; | Anti-mullerian hormone | GM-102 |
| antigen CD20 | obinituzumab; | II (MHR2) | |
| | ocaratuzumab | | |
| Membrane glycol- | samalizumab | MMP1/HLA (MMP1 | Anti-MMP1/HLA |
| protein OX2 CD200 | | peptide presented in | mAbs |
| | | MHC1) | |
| CD22 | epratuzumab | Metalloprotease-9 | andecaliximab |
| | | (MMP9) | |
| Immunoglobulin | lumiliximab | Mesothelin (MSLN) | MORAb-009 |
| epsilon Fc receptor | | | |
| II (CD23) | | | |
| Signal transducer | anti-CD24 mAbs | Mucin 1 (MUC1) | PankoMab-GEX |
| CD24 | | | |
| IL-2 receptor alpha | 90Y-daclizumab | Mucin 13 (MUC13) | anti-MUC13 mAbs |
| subunit CD25 | | | |
| CD27 | varilumab | Endomucin (MUC14) | anti-MUC14 mAbs |
| CD28 | theralizumab | Mucin 16 (MUC16) | sofituzumab |
| CD3 | Muromonab- | Cell surface glycoprotein | AA98 |
| | CD3 (OKT3) | MUC18 (CD146) | |
| CD30 | brentuximab | Mucin 5AC (MUC5AC) | ensituximab |
| | vedotin | | |
| Immunoglobulin | BI-1206 | N-glycolyl GM3 | 99mTc-labeled |
| gamma Fc receptor | | (NeuGcGM3) | 14F7 |
| IIB (CD32B) | | | |
| CD33 | lintuzumab | Sodium-dependent | XMT-1536 |
| | | phosphate transport | |
| | | protein 2B (SLC34A2) | |
| CD37 | otlertuzumab | Nucleolin (NCL) | anti-nucleolin |
| | | | mAbs |
| ADP ribosyl | daratumumab | Nectin-4 | enfortumab vedotin |
| cyclase-1 (CD38) | | | |
| CD39 | OREG-103 | Neurofibromin (NF1) | anti-neurofibromin |
| | | | mAbs |
| CD4 | IT-1208 | NGcGM3 ganglioside | racotumomab |
| CD40 | lucatumumab | NKG2A | monalizumab |
| CD43 | leukotuximab | non-POU domain- | PAT-LM1 |
| | | containing octamer- | |
| | | binding protein (NONO) | |
| CD44 | RG7356 | Notch-1 | brontictuzumab |
| CD45 | 131I-BC8 | CD73 | oleclumab |
| Membrane cofactor | AugmAb | Netrin-1 (NTN1) | NP-137 |
| protein (CD46) | | | |
| CD47 | Hu5F9-G4 | OX-40 | PF-04518600 |
| CD52 | alemtuzumab | P2X purinoceptor 7 | BIL-010t |
| | | (P2RX7) | |
| CD55 | PAT-SC1 | FGF receptor (pan | MM-161 |
| | | FGFR) | |
| Neural cell adhesion | IMGN-901 | Integrin (Pan integrin) | NOD201 |
| molecule 1; (CD56) | | | |
| T-cell differentiation | itolizumab | P-cadherin, also | PCA-062 |
| antigen CD6 | | cadherin-3 (CDH3) | |
| CD70 | SGN-70 | Programmed cell death | pembrolizumab |
| | | protein 1 (PD-1) | |
| CD79b | polatuzumab | Programmed cell death | avelumab; Euchloe |
| | vedotin | ligand 1 (PD-L1) | H12 |
| CD8 | anti-CD8 mAbs | Programmed cell death | rHIgM12B7 |
| | | ligand 2 (PD-L2) | |
| CD80 | galiximab | PDGF receptor alpha | olaratumumab |
| | | (PDGFRA) | |
| CD98 | IGN-523 | Placenta specific protein | anti-PLAC1 mAbs |
| | | 1 (PLAC1) | |
| CD99 | NV-103 | PR1/HLA (PR1 peptide | anti-PR1/HLA |
| | | in MHC) | mAbs |
| Cadherin-1 (CDH1) | anti-CDH1 | Prolactin receptor PRLR | ABBV-176 |
| | mAbs | | |
| Cadherin-17 | anti-CDH17 | Phosphatidylserine | anti- |
| (CDH17) | mAbs | | phosphatidylserine |
| | | | mAbs |
| Cadherin 19 | anti-CDH19 | Prostate stem cell antigen | anti-PSCA mAbs |
| (CDH19) | mAbs | (PSCA) | |
| Cadherin-6 (CDH6) | HKT-288 | Glutamate | ATL-101 |
| | | carboxypeptidase II | |
| | | (PSMA) | |
| CD66a (CEACAM1) | CM-24 | Parathyroid hormone- | CAL |
| | | related protein (PTH-rP) | |
| CD66e (CEACAM5) | IMMU-130 | Tyrosine-protein kinase- | cofetuzumab |
| | | like 7 (PTK7) | pelidotin |
| CD66c; CD66e | NEO-201 | Protein tyrosine | PRL3-zumab |
| (CEACAM5/6) | | phosphatase IVA3 | |
| | | (PTP4A3) | |
| Claudin 18 (Claudin | IMAB362 | Poliovirus receptor | COM-701 |
| 18.2) | | related immunoglobulin | |
| | | domain containing | |
| | | (PVRIG) | |
| Claudin 6 | IMAB027 | Receptor activator of | denosumab |
| | | nuclear factor kappa- | |
| | | B ligand (RANKL) | |
| SLAM family | elotuzumab | Recepteur d'origine | anti-RON mAbs |
| member 7 (CS1) | | nantais (RON) | |
| colony stimulating | cabiralizumab | Tyrosine-protein kinase | cirmtuzumab |
| factor-1 receptor | | transmembrane receptor | |
| (CSF1R) | | ROR1 (ROR1); also | |
| | | NTRKR1 | |
| Cytotoxic T- | ipilumumab | Tyrosine-protein kinase | BA-3021 |
| lymphocyte protein- | | transmembrane receptor | |
| 4 (CTLA4) | | ROR2 (ROR2); also | |
| | | NTRKR2 | |
| Coxsackievirus and | anti-CXADR | R-spondin-3 (RSPO3) | rosmantuzumab |
| adenovirus receptor | mAbs | | |
| (CXADR) | | | |
| CXCR2 chemokine | anti-CXCR2 | Sphingosine-1-phosphate | EDD7H9 |
| receptor | mAbs | receptor 3 (S1PR3) | |
| CXCR3 chemokine | anti-CXCR3 | Surface Antigen In | IGN-786 |
| receptor | mAbs | Leukemia (SAIL) | |
| CXCR4 chemokine | ulocuplumab | Semaphorin-4D | VX-15 |
| receptor | | (SEMA4D) | |
| CXCR5 chemokine | STI-B030X | carbohydrate antigen 19- | MVT-1075 |
| receptor | | 9 (CA 19-9) | |
| CXCR7 chemokine | anti-CXCR7 | Sialyl Thomsen nouveau | anti-STn mAbs |
| receptor | mAbs | antigen (STn) | |
| DCLK1 | anti-DCLK1 | Sialic acid-binding Ig- | AK-002 |
| | mAbs | like lectin 8 (Siglec-8) | |
| Dickkopf-related | BHQ-880 | Sialic acid-binding Ig- | anti-Siglec-9 mAbs |
| protein 1 (DKK1) | | like lectin 9 (Siglec-9) | |
| DLK1 | ADCT-701 | Signal Regulatory | OSE-172 |
| | | Protein Alpha (SIRPA) | |
| Delta-like protein | SC16LD6.5 | CD48; also SLAM | SGN-CD48A |
| ligand 3 (DLL3) | | family member 2 | |
| | | (SLAMF2) | |
| Delta-like protein | navicixizumab | CD352; SLAM family | SGN-CD352A |
| ligand 4 (DLL4); | | member 6 (SLAMF6) | |
| VEGF (VEGF) | | | |
| Dipeptidyl | YSCMA | Neutral amino acid | KM-8094 |
| peptidase-4 (DPP4), | | transporter B0 (SLC1A5) | |
| (also CD26) | | | |
| Death receptor-3 | PTX-35 | Somatostatin 2 receptor | XmAb-18087 |
| (DR3) | | (SSTR2) | |
| TRAIL-1 receptor | HuYON007 | Stabilin 1 (STAB1) | FP-1305 |
| (DR4) | MultYbody | | |
| TRAIL-1 receptor; | DR4/DR5 | Metalloreductase | 89Zr-DFO- |
| TRAIL-2 receptor | Surrobody | (STEAP1) | MSTP2109A |
| (DR4/DR5) | | | |
| TRAIL-2 receptor | DS-8273 | Survivin | anti-survivin mAbs |
| (DR5) | | | |
| EGF-like protein 6 | anti-EGFL6 | TAG-72 | 90Y-IDEC-159 |
| (EGFL6) | mAbs | | |
| Epidermal growth | cetuximab; | T cell receptor (TCR) | anti-TCR mAbs |
| factor receptor | Sym004; | | |
| (EGFR) | nimotuzumab | | |
| Epidermal growth | ABT-806 | Endosialin (TEM1) | ontuxizumab |
| factor receptor vIII | | | |
| (EGFRvIII) | | | |
| Epithelial membrane | ONCR-201 | Anthrax toxin receptor 1 | anti-TEM8 mAbs |
| protein 2 (EMP2) | | (ANTXR1); also TEM8 | |
| Endoglin | carotuximab | Tissue factor (TF) | MORAb-066 |
| Ectonucleotide | AGS-16C3F | Transforming growth | anti-TGFBR2 |
| pyrophosphatase/ | | factor, beta receptor II | mAbs |
| phosphodiesterase | | TGF-beta type II | |
| family member 3 | | (TGFBR2) | |
| (ENPP3) | | | |
| Prostaglandin E₂ | anti-PTGER2 | Thomsen-Friedenreich | JAA-F11 |
| receptor 2 | mAbs | Antigen | |
| (PTGER2) | | | |
| Prostaglandin E₂ | anti-PTGER4 | T cell immunoreceptor | BMS-986207 |
| receptor 4 | mAbs | with Ig and ITIM | |
| (PTGER4) | | domains (TIGIT) | |
| EpCAM | oportuzumab | Hepatitis A virus cellular | CDX-014 |
| | monatox | receptor 1 (HAVCR1); | |
| | | also TIM-1 | |
| Ephrin type-A | MEDI-547 | Hepatitis A virus cellular | MBG453 |
| receptor 2 (EphA2) | | receptor 2 (HAVCR2); | |
| | | also TIM-3 | |
| Ephrin type-A | KB004 | Toll-like receptor 2 | OPN-305 |
| receptor 3 (EphA3) | | (TLR-2) | |
| Fibroblast activation | F19 | Toll-like receptor 4 | anti-TLR4 mAbs |
| protein (FAP) | | (TLR-4) | |
| CD95 (FAS) | asunercept | Transmembrane 4 L6 | anti-TM4SF1 |
| | | family member 1 | mAbs |
| | | (TM4SF1) | |
| Fc receptor like | RG-6160 | Tumor necrosis factor | anti-TNFR2 mAbs |
| protein 5 (FCRL5) | | receptor 2 (TNFR2) | |
| FGF receptor 1 | FP-1039 | CD71 | anti-CD71 mAbs |
| (FGFR1) | | | |
| FGF receptor 2b | FPA-144 | Triggering receptor | anti-TREM1 mAbs |
| (FGFR2b) | | expressed on myeloid | |
| | | cells 1 (TREM1) | |
| FGF receptor 3 | B-701 | Tumor-associated | DS-1062 |
| (FGFR3) | | calcium signal transducer | |
| | | 2 (Trop-2) | |
| fms-like tyrosine | Flysyn | TWEAK Receptor | MRT-101 |
| kinase 3 (FLT3) | | (TWEAKR) | |
| Folate receptor alpha | farletuzumab; | Tyrosine-protein kinase | ELB-031 |
| (FOLR1) | IMGN853; | receptor TYRO3 | |
| | KHK2805 | (TYRO3) | |
| Folate receptor beta | anti-FOLR beta | Urokinase receptor | MNPR-101 |
| (FOLR2) | mAbs | (uPAR) | |
| Frizzled-1; Frizzled- | vantictumab | VEGF-2 (VEGFR2) | ramucirumab |
| 2; Frizzled-5; | | | |
| Frizzled-7; Frizzled- | | | |
| 8; (FZD1, 2, 5, 7, 8) | | | |
| Follistatin-like | anti-FSTL1 | Vimentin | pritumumab |
| protein 1 (FSTL1) | mAbs | | |
| Fucosyl-GM1 | BMS-986012 | V-domain Ig suppressor | JNJ-61610588 |
| | | of T cell activation | |
| | | (VISTA) | |
| Frizzled-10 (FZD10) | OTSA-101 | Integrin alpha-4/beta-1 | natalizumab |
| GCSF-R (Also, | CSL324 | Immunoglobulin iota | anti-VPREB1 |
| CD114 and CSFR3) | | chain (VPREB1) | mAbs |
| Galectin 3 binding | MP-1959 | Wilms tumor protein | ESK1 |
| protein (LGALS3) | | (WT1/HLA); WT1 | |
| | | peptide presented in | |
| | | MHC | |
| Guanylate cyclase | TAK-164 | Glypican-3 (GPC3) | codrituzumab |
| 2C (GUCY2C) | | | |
| GD2 | dinutuximab | Transmembrane | CDX-011 |
| | | glycoprotein NMB | |
| | | (GPNMB) | |
| GD3 | PF-06688992 | Leucine-rich repeat- | BNC-101 |
| | | containing G-protein | |
| | | coupled receptor 5 | |
| | | (LGR5) | |
| glucocorticoid- | BMS-986156 | G-protein coupled | JNJ-64407564 |
| induced TNFR- | | receptor family C group | |
| related protein | | 5 member D (GPRC5D) | |
| (GITR) | | | |
| glucocorticoid- | EU-102 | Ferritin | Ferritarg P |
| induced TNFR- | | | |
| related protein | | | |
| ligand (GITRL) | | | |
| premelanocyte | anti-PMEL | Erbb2 tyrosine kinase | trastuzumab; |
| protein (PMEL) | mAbs | (HER2) | pertuzumab; |
| | | | margetuximab |
| Cell surface A33 | Anti-GPA33 | Erbb3 tyrosine kinase | patritumab |
| antigen (GPA33) | mAbs | (HER3) | |
| Glypican-1 (GPC1) | MIL-38 | Globo H | OBI-888 |

The extracellular antigen binding domain may comprise an antigen binding fragment (e.g., an scFv) derived from any of the antibodies listed in Table 4 depending upon the target antigen of interest. In some embodiments, the antigen binding fragment (e.g., an scFv) may comprise the same heavy chain and light chain complementarity determining regions (CDRs) as the antibodies listed in Table 4 depending upon the target antigen of interest. In some examples, the antigen binding fragment (e.g., an scFv) may comprise the same heavy chain variable region (VH) and light chain variable region VL as the antibodies listed in Table 4 depending upon the target antigen of interest.

In other embodiments, the extracellular antigen binding domain of any of the CAR polypeptides described herein may be specific to a pathogenic antigen, such as a bacterial antigen, a viral antigen, or a fungal antigen. Some examples are provided below: influenza virus neuraminidase, hemagglutinin, or M2 protein, human respiratory syncytial virus (RSV) F glycoprotein or G glycoprotein, herpes simplex virus glycoprotein gB, gC, gD, or gE, Chlamydia MOMP or PorB protein, Dengue virus core protein, matrix protein, or glycoprotein E, measles virus hemagglutinin, herpes simplex virus type 2 glycoprotein gB, poliovirus I VP1, envelope glycoproteins of HIV 1, hepatitis B core antigen or surface antigen, diptheria toxin, Streptococcus 24M epitope, Gonococcal pilin, pseudorabies virus g50 (gpD), pseudorabies virus II (gpB), pseudorabies virus III (gpC), pseudorabies virus glycoprotein H, pseudorabies virus glycoprotein E, coronavirus polypeptides, transmissible gastroenteritis glycoprotein 195, transmissible gastroenteritis matrix protein, human papilloma virus E6 or E7, or human hepatitis C virus glycoprotein E1 or E2.

In addition, the extracellular antigen binding domain of the CAR polypeptide described herein may be specific to a tag conjugated to a therapeutic agent, which targets an antigen associated with a disease or disorder (e.g., a tumor antigen or a pathogenic antigen as described herein). In some instances, the tag conjugated to the therapeutic agent can be antigenic and the extracellular antigen binding domain of the CAR polypeptide can be an antigen-binding fragment (e.g., scFv) of an antibody having high binding affinity and/or specificity to the antigenic tag. Exemplary antigenic tags include, but are not limited to, biotin, avidin, a fluorescent molecule (e.g., GFP, YRP, luciferase, or RFP), Myc, Flag, His (e.g., poly His such as 6×His), HA (hemeagglutinin), GST, MBP (maltose binding protein), KLH (keyhole limpet hemocyanins), trx, T7, HSV, VSV (e.g., VSV-G), Glu-Glu, V5, e-tag, S-tag, KT3, E2, Au1, Au5, and/or thioredoxin.

In other instances, the tag conjugated to the therapeutic agent is a member of a ligand-receptor pair and the extracellular antigen binding domain comprises the other member of the ligand-receptor pair or a fragment thereof that binds the tag. For example, the tag conjugated to the therapeutic agent can be biotin and the extracellular antigen binding domain of the CAR polypeptide can comprise a biotin-binding fragment of avidin. See, e.g., (Urbanska et al., Cancer Res, 72(7): 1844-1852 (2012); Lohmueller et al., Oncoimmunology, 7(1): e1368604 (2017)). Other examples include anti-Tag CAR, in which the extracellular antigen binding domain is a scFv fragment specific to a protein tag, such as FITC (Tamada et al., Clin Cancer Res, 18(23): 6436-6445 (2012); Kim et al., J Am Chem Soc, 137(8): 2832-2835 (2015); Cao et al., Angew Chem Int 25 Ed Engl, 55(26): 7520-7524 (2016); Ma et al., Proc Natl Acad Sci USA, 113(4): E450-458 (2016)), PNE (Rodgers et al., Proc Natl Acad Sci USA, 113(4): E459-468 (2016)), La-SS-B (Cartellieri et al., Blood Cancer J, 6(8): e458 (2016)), Biotin (Lohmueller, Ham et al., Oncoimmunology, 7(1): e1368604 (2017)) and Leucine-Zipper (Cho et al., Cell, 173(6): 1426-1438.e1411 (2018)). Selection of the antigen binding domain for use in the CAR polypeptides described herein will be apparent to one of skill in the art. For example, it may depend on factors such as the type of target antigen and the desired affinity of the binding interaction.

The extracellular antigen binding domain of any of the CAR polypeptides described herein may have suitable binding affinity for a target antigen (e.g., any one of the targets described herein) or antigenic epitopes thereof. As used herein, “binding affinity” refers to the apparent association constant or K_{A} or the K_{D}. The K_{A} is the reciprocal of the dissociation constant (K_{D}). The extracellular antigen binding domain for use in the CAR polypeptides described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Higher affinity binding of an extracellular antigen binding domain for a first antigen relative to a second antigen can be indicated by a higher K_{A} (or a smaller numerical value K_{D}) for binding the first antigen than the K_{A} (or numerical value K_{D}) for binding the second antigen. In such cases, the extracellular antigen binding domain has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 100,000-fold.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH 7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

$$[ Bound ] = [ Free ] / ( Kd + [ Free ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

### B. Transmembrane Domain

The transmembrane domain of the chimeric receptor polypeptides (e.g., ACTR polypeptides or CAR polypeptides) described herein can be in any form known in the art. As used herein, a “transmembrane domain” refers to any protein structure that is thermodynamically stable in a cell membrane, preferably a eukaryotic cell membrane. A transmembrane domain compatible for use in the chimeric receptor polypeptides used herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three-dimensional structure of the transmembrane domain. For example, transmembrane domains may form an alpha helix, a complex of more than one alpha helices, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type II depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type II membrane proteins have multiple membrane-spanning segments and may be further sub-classified based on the number of transmembrane segments and the location of N- and C-terminus.

In some embodiments, the transmembrane domain of the chimeric receptor polypeptide described herein is derived from a Type I single-pass membrane protein. Preferably, the transmembrane domain is of a membrane protein selected from the group consisting of CD8α, CD8β, 4-1BB/CD137, CD27, CD28, CD34, CD4, FcεRIγ, CD16A, OX40/CD134, CD3ζ, CD3ε, CD3γ, CD3δ, TCRα, TCRβ, TCRζ, CD32, CD64, CD45, CD5, CD9, CD22, CD37, CD80, CD86, CD40, CD40UCD154, VEGFR2, FAS, FGFR2B, CD2, IL15, IL15R, IL21, DNAM-1, 2B4, NKG2D, NKp44 and NKp46. In some embodiments, the transmembrane domain is from a membrane protein selected from the following: CD8a, CD8b, 4-1BB, CD28, CD34, CD4, FcεRIγ, CD16A, OX40, CD3z, CD3e, CD3g, CD3d, TCRα, CD32, CD64, VEGFR2, FAS, FGFR2B, DNAM-1, 2B4, NKG2D, NKp44 and NKp46. In some examples, the transmembrane domain is of CD8 (e.g., the transmembrane domain is of CD8α). In some examples, the transmembrane domain is of 4-1BB/CD137. In other embodiments, the transmembrane domain is of CD28. In other embodiments, the transmembrane domain is of NKG2D, NKp44 or NKp46. In other examples, the transmembrane domain is of CD34. In yet other examples, the transmembrane domain is not derived from human CD8a. In some embodiments, the transmembrane domain of the chimeric receptor polypeptide is a single-pass alpha helix.

The amino acid sequences of exemplary transmembrane domains are provided in Table 5:

TABLE 5
Exemplary Transmembrane Domains

| Trans- | | |
|---|---|---|
| membrane | | |
| domain | Sequences | SEQ ID NO. |
| CD8α | DIYIWAPLAGTCGVLLLSLVITLYC | SEQ ID NO: 18 |
| 4-1BB/ | DIISFFLALTSTALLFLLFFLTLRF | SEQ ID NO: 19 |
| CD137 | SVV | |
| CD28 | DFWVLVVVGGVLACYSLLVTVAFII | SEQ ID NO: 20 |
| | FWVRS | |
| CD34 | DLIALVTSGALLAVLGITGYFLMNR | SEQ ID NO: 21 |
| Designed | DLLAALLALLAALLALLAALLARSK | SEQ ID NO: 22 |
| hydro- | | |
| phobic | | |
| CD4 | DMALIVLGGVAGLLLFIGLGIFFCV | SEQ ID NO: 23 |
| | R | |
| FCεRIγ | DLCYILDAILFLYGIVLTLLYCRLK | SEQ ID NO: 24 |
| Designed | DLLLILLGVLAGVLATLAALLARSK | SEQ ID NO: 25 |
| hydro- | | |
| phobic, | | |
| predicted | | |
| dimeri- | | |
| zation | | |
| CD8β | DITLGLLVAGVLVLLVSLGVAIHLC | SEQ ID NO: 26 |
| CD16α | DVSFCLVMVLLFAVDTGLYFSVKTN | SEQ ID NO: 27 |
| OX40/ | DVAAILGLGLVLGLLGPLAILLALY | SEQ ID NO: 28 |
| CD134 | | |
| CD3ε | DVMSVATIVIVDICITGGLLLLVYY | SEQ ID NO: 29 |
| | WSKN | |
| CD3ζ | DLCYLLDGILFIYGVILTALFLRVK | SEQ ID NO: 30 |
| CD3γ | DGFLFAEIVSIFVLAVGVYFIAGQD | SEQ ID NO: 31 |
| CD3δ | DGIIVTDVIATLLLALGVFCFAGHE | SEQ ID NO: 32 |
| | T | |
| TCR-α | DVIGFRILLLKVAGFNLLMTLRLW | SEQ ID NO: 33 |
| CD32 | DIIVAVVIATAVAAIVAAVVALIYC | SEQ ID NO: 34 |
| | RK | |
| CD64 | DVLFYLAVGIMFLVNTVLWVTIRKE | SEQ ID NO: 35 |
| VEGFR2 | DIIILVGTAVIAMFFWLLLVIILRT | SEQ ID NO: 36 |
| FAS | DLGWLCLLLLPIPLIVWVKRK | SEQ ID NO: 37 |
| FGFR2B | DIAIYCIGVFLIACMVVTVILCRMK | SEQ ID NO: 38 |
| CD8 + 4aa | FACDIYIWAPLAGTCGVLLLSLVIT | SEQ ID NO: 39 |
| | LYC | |

Transmembrane domains from multi-pass membrane proteins may also be compatible for use in the chimeric receptor polypeptides described herein. Multi-pass membrane proteins may comprise a complex alpha helical structure (e.g., at least 2, 3, 4, 5, 6, 7 or more alpha helices) or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. In some instances, the reverse orientation of such a native transmembrane protein may be constructed for efficient orientation of the chimeric receptor polypeptide (e.g., CAR) within the immune cell membrane. Either one or multiple helices passes from a multi-pass membrane protein can be used for constructing the chimeric receptor polypeptide described herein.

Transmembrane domains for use in the chimeric receptor polypeptides described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906B1 and WO 2000/032776A2, the relevant disclosures of each of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5, or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, hydrophobic or hydrophilic characteristics of a protein or protein segment, can be assessed by any method known in the art including, for example, the Kyte and Doolittle hydropathy analysis.

### C. Co-Stimulatory Signaling Domains

Many immune cells (e.g., NK or T cells) require co-stimulation, in addition to stimulation of an antigen-specific signal, to promote cell proliferation, differentiation and survival, as well as to activate effector functions of the cell. The term “co-stimulatory signaling domain”, as used herein, refers to at least a fragment of a co-stimulatory signaling protein that mediates signal transduction within a cell to induce an immune response such as an effector function (a secondary signal). As known in the art, activation of immune cells such as T cells often require two signals: (1) the antigen specific signal (primary signal) triggered by the engagement of T cell receptor (TCR) and antigenic peptide/MHC complexes presented by antigen presenting cells, which typically is driven by CD3ζ as a component of the TCR complex; and (ii) a co-stimulatory signal (secondary signal) triggered by the interaction between a co-stimulatory receptor and its ligand. A co-stimulatory receptor transduces a co-stimulatory signal (secondary signal) as an addition to the TCR-triggered signaling and modulates responses mediated by immune cells, such as T cells, NK cells, macrophages, neutrophils, or eosinophils.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the chimeric receptor polypeptides described herein. The type(s) of co-stimulatory signaling domain is selected based on factors such as the type of the immune cells in which the chimeric receptor polypeptides would be expressed (e.g., T cells, NK cells, macrophages, neutrophils, or eosinophils) and the desired immune effector function (e.g., ADCC). Accordingly, it is one embodiment that the chimeric receptor polypeptide of the genetically engineered immune cell comprises the at least one co-stimulatory signaling domain. Examples of co-stimulatory signaling domains for use in the chimeric receptor polypeptides may be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFRSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/FNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4/CD2525, RELTiTNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP10, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2D, NKG2C, NKp30, NKp44, NKp46 and JAMAL. In certain embodiments, the chimeric receptor polypeptides may contain a CD28 co-stimulatory signaling domain or a 4-1BB (CD137) co-stimulatory signaling domain. In some embodiments, at least one co-stimulatory signaling domain is selected from the group consisting of 4-1BB, CD28, CD8α, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL or any variant thereof.

Also within the scope of the present disclosure are functional variants of any of the co-stimulatory signaling domains described herein, such that the co-stimulatory signaling domain is capable of modulating the immune response of the immune cell. In some embodiments, the co-stimulatory signaling domains comprises up to 10 amino acid residue mutations (e.g., 1, 2, 3, 4, 5, or 8) such as amino acid substitutions, deletions, or additions as compared to a wild-type counterpart. Such co-stimulatory signaling domains comprising one or more amino acid variations (e.g., amino acid substitutions, deletions, or additions) may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the chimeric receptor polypeptide. In some embodiments, the mutations are substitution of a lysine at each of positions 186 and 187 with a glycine residue of the CD28 co-stimulatory domain, referred to as a CD28_{LL→GG} variant.

Therefore, a suitable variant of CD28 is the CD28_{LL→GG} variant.

Additional mutations can be made in co-stimulatory signaling domains that may enhance or reduce co-stimulatory activity of the domain will be evident to one of ordinary skill in the art. In some embodiments, the co-stimulatory signaling domain is selected from the group of 4-1BB, CD28, OX40, and CD28_{LL→GG} variant. In one embodiment, the at least one co-stimulatory signaling domains is a CD28 co-stimulatory signaling domain or a 4-1BB co-stimulatory signaling domain.

In some embodiments, the chimeric receptor polypeptides may contain a single co-stimulatory domain such as, for example, a CD27 co-stimulatory domain, a CD28 co-stimulatory domain, a 4-1BB co-stimulatory domain, an ICOS co-stimulatory domain, an OX40 co-stimulatory domain, an OX40L co-stimulatory domain, a 2B4 co-stimulatory domain, a GITR co-stimulatory domain, a NKG2D co-stimulatory domain, a NKp30 co-stimulatory domain, a NKp44co-stimulatory domain, a NKp46 co-stimulatory domain, a DAP10 co-stimulatory domain, a DAP12 co-stimulatory domain, a DNAM1 co-stimulatory domain, a LFA-1 co-stimulatory domain, a HVEM co-stimulatory domain or a JAMAL co-stimulatory domain.

Selection of the type(s) of co-stimulatory signaling domains may be based on factors such as the type of host cells to be used with the chimeric receptor polypeptides (e.g., αβ T, γδ T or NK cells) and the desired immune effector function.

In some embodiments, the chimeric receptor polypeptides may comprise more than one co-stimulatory signaling domain (e.g., 2, 3, or more). In one embodiment, the chimeric receptor polypeptide comprises at least two co-stimulatory signaling domains. In one preferred embodiment, the chimeric receptor polypeptide comprises two co-stimulatory signaling domains. In some embodiments, the chimeric receptor polypeptide comprises two or more of the same co-stimulatory signaling domains, for example, two copies of the co-stimulatory signaling domain of CD28. In some embodiments, the chimeric receptor polypeptide comprises two or more co-stimulatory signaling domains from different co-stimulatory proteins, such as any two or more co-stimulatory proteins described herein. In some embodiments, the chimeric receptor polypeptide may comprise two or more co-stimulatory signaling domains from different co-stimulatory receptors, such as any two or more co-stimulatory receptors described herein, for example, CD28 and 4-1BB, CD28 and CD27, CD28 and ICOS, CD28_{LL→GG} variant and 4-1BB, CD28 and OX40, or CD28_{LL→GG} variant and OX40. In some embodiments, the two co-stimulatory signaling domains are CD28 and 4-1BB. In some embodiments, the two co-stimulatory signaling domains are CD28_{LL→GG} variant and 4-1BB. In some embodiments, the two co-stimulatory signaling domains are CD28 and OX40. In some embodiments, the two co-stimulatory signaling domains are CD28_{LL→GG} variant and OX40. In some embodiments, the chimeric receptor polypeptides described herein may contain a combination of a CD28 and ICOSL. In some embodiments, the chimeric receptor polypeptide described herein may contain a combination of CD28 and CD27. In certain embodiments, the 4-1BB co-stimulatory domain is located N-terminal to the CD28 or CD28_{LL→GG} variant co-stimulatory signaling domain.

In some embodiments, one of the co-stimulatory signaling domains is a CD28 co-stimulatory signaling domain and the other co-stimulatory domain is selected from the group consisting of a CD8α, 4-1BB, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain. In some embodiments, one of the co-stimulatory signaling domains is a CD8α co-stimulatory signaling domain and the other co-stimulatory domain is selected from the group consisting of a CD28, 4-1BB, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain. In some embodiments, one of the co-stimulatory signaling domains is a 4-1BB co-stimulatory signaling domain and the other co-stimulatory domain is selected from the group consisting of a CD8α, CD28, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain.

In some embodiments, the chimeric receptor polypeptides described herein do not comprise a co-stimulatory signaling domain.

The amino acid sequences of exemplary co-stimulatory domains are provided in Table 6.

TABLE 6
Exemplary Co-Stimulatory Domains

| Co-stimulatory | | |
|---|---|---|
| domain | Sequences | SEQ ID NO. |
| 4-1BB/CD137 | KRGRKKLLYIFKQPFMRPVQTTQEEDGCSCR | SEQ ID NO: 40 |
| | FPEEEEGGCEL | |
| CD28 | RSKRSRLLHSDYMNMTPRRPGPTRKHYQPYA | SEQ ID NO: 41 |
| | PPRDFAAYRS | |
| OX40/CD134 | ALYLLRRDQRLPPDAHKPPGGGSFRTPIQEE | SEQ ID NO: 42 |
| | QADAHSTLAKI | |
| OX40L/CD252 | ERVQPLEENVGNAARPRFERNK | SEQ ID NO: 43 |
| ICOS | KKKYSSSVHDPNGEYMFMRAVNTAKKSRLTD | SEQ ID NO: 44 |
| | VTL | |
| CD27 | QRRKYRSNKGESPVEPAEPCHYSCPREEEGS | SEQ ID NO: 45 |
| | TIPIQEDYRKPEPACSP | |
| GITR | QLGLHIWQLRSQCMWPRETQLLLEVPPSTED | SEQ ID NO: 46 |
| | ARSCQFPEEERGERSAEEKGRLGDLWV | |
| HVEM | CVKRRKPRGDVVKVIVSVQRKRQEAEGEATV | SEQ ID NO: 47 |
| | IEALQAPPDVTTVAVEETIPSFTGRSPNH | |
| TIM1 | KKYFFKKEVQQLSVSFSSLQIKALQNAVEKE | SEQ ID NO: 48 |
| | VQAEDNIYIENSLYATD | |
| LFA1/CD11a | DIYIWAPLAGTCGVLLLSLVITLYCYKVGFF | SEQ ID NO: 49 |
| | KRNLKEKMEAGRGVPNGIPAEDSEQLASGQE | |
| | AGDPGCLKPLHEKDSESGGGKD | |
| CD2 | DIYIWAPLAGTCGVLLLSLVITLYCKRKKQR | SEQ ID NO: 50 |
| | SRRNDEELETRAHRVATEERGRKPHQIPAST | |
| | PQNPATSQHPPPPPGHRSQAPSHRPPPPGHR | |
| | VQHQPQKRPPAPSGTQVHQQKGPPLPRPRVQ | |
| | PKPPHGAAENSLSPSSN | |
| CD28 + CD27 | RSKRSRLLHSDYMNMTPRRPGPTRKHYQPYA | SEQ ID NO: 51 |
| | PPRDFAAYRSQRRKYRSNKGESPVEPAEPCH | |
| | YSCPREEEGSTIPIQEDYRKPEPACSP | |
| CD28+ OX40 | RSKRSRLLHSDYMNMTPRRPGPTRKHYQPYA | SEQ ID NO: 52 |
| | PPRDFAAYRSRRDQRLPPDAHKPPGGGSFRT | |
| | PIQEEQADAHSTLAKI | |
| 4-1BB + CD28 | KRGRKKLLYIFKQPFMRPVQTTQEEDGCSCR | SEQ ID NO: 53 |
| | FPEEEEGGCELRSKRSRLLHSDYMNMTPRRP | |
| | GPTRKHYQPYAPPRDFAAYRS | |
| CD28 + 4-1BB | RSRLLHSDYMNMTPRRPGPTRKHYQPYAPPR | SEQ ID NO: 54 |
| | DFAAYRSKRGRKKLLYIFKQPFMRPVQTTQE | |
| | EDGCSCRFPEEEEGGCEL | |
| CD28 + ICOS | RSKRSRLLHSDYMNMTPRRPGPTRKHYQPYA | SEQ ID NO: 55 |
| | PPRDFAAYRSKKKYSSSVHDPNGEYMFMRAV | |
| | NTAKKSRLTDVTL | |
| CD28LL to GG | RSKRSRGGHSDYMNMTPRRPGPTRKHYQPYA | SEQ ID NO: 56 |
| mutant | PPRDFAAYRS | |
| CD28LL to GG | RSKRSRGGHSDYMNMTPRRPGPTRKHYQPYA | SEQ ID NO: 57 |
| mutant + 4-1BB | PPRDFAAYRSKRGRKKLLYIFKQPFMRPVQT | |
| | TQEEDGCSCRFPEEEEGGCEL | |
| DAP10 | CARPRRSPAQEDGKVYINMPGRG | SEQ ID NO: 58 |
| DNAM-1 | NRRRRRERRDLFTESWDTQKAPNNYRSPIST | SEQ ID NO: 59 |
| | SQPTNQSMDDTREDIYVNYPTFSRRPKTRV | |
| NKp30 | GSTVYYQGKCLTWKGPRRQLPAVVPAPLPPP | SEQ ID NO: 60 |
| | CGSSAHLLPPVPGG | |
| NKp44 | WWGDIWWKTMMELRSLDTQKATCHLQQVTDL | SEQ ID NO: 61 |
| | PWTSVSSPVEREILYHTVARTKISDDDDEHT | |
| | L | |

Alternatively, any of the chimeric receptor polypeptide may be free of any co-stimulatory signaling domain.

### D. Cytoplasmic Signaling Domain

Any cytoplasmic signaling domain can be used to create the chimeric receptor polypeptides described herein (e.g., ACTR polypeptides or CAR polypeptides). Such a cytoplasmic domain may be any signaling domain involved in triggering cell signaling (primary signaling) that leads to immune cell proliferation and/or activation. The cytoplasmic signaling domain as described herein is not a co-stimulatory signaling domain, which, as known in the art, relays a co-stimulatory or secondary signal for fully activating immune cells (e.g., CAR-T).

The cytoplasmic signaling domain described herein may comprise an immunoreceptor tyrosine-based activation motif (ITAM) domain (e.g., at least one iTAM domain, at least two iTAM domains, or at least three iTAM domains) or may be ITAM free. An “iTAM,” as used herein, is a conserved protein motif that is generally present in the tail portion of signaling molecules expressed in many immune cells. The motif may comprise two repeats of the amino acid sequence YxxL/I separated by 6-8 amino acids, wherein each x is independently any amino acid, producing the conserved motif YxxL/Ix₍₆₋₈₎YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Examples of ITAMs for use in the chimeric receptor polypeptides comprised within the cytoplasmic signaling domain, without limitation may be: CD3γ, CD3ε, CD3δ, each containing a single ITAM motif while each ζ chain contains 3 distinct ITAM domains (ζa, ζb and ζc. The number and ITAM sequences are also important in the design of CARs (Bettini et al., J Immunol, 199(5): 1555-1560 (2017); Jayaraman et al., EBioMedicine, 58: 102931 (2020)).

In some embodiments, the cytoplasmic signaling domain is of CD3ζ or FcεR1γ. The amino acid sequence of one exemplary cytoplasmic signaling domain of human CD3z is provided below:

(SEQ ID NO: 66)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In other examples, cytoplasmic signaling domain is not derived from human CD3ζ. In yet other examples, the cytoplasmic signaling domain is not derived from an Fc receptor, when the extracellular Fc-binding domain of the same chimeric receptor polypeptide is derived from CD16A.

In one specific embodiment, several signaling domains can be fused together for additive or synergistic effect. Non-limiting examples of useful additional signaling domains include part or all of one or more of TCRζ chain, CD28, OX40/CD134, 4-1BB/CD137, FcεRIγ, ICOS/CD278, IL2Rα/CD122, IL-2Rγ/CD132, and CD40.

In other embodiments, the cytoplasmic signaling domain described herein is free of the ITAM motif. Examples include, but are not limited to, the cytoplasmic signaling domain of Jak/STAT, Toll-interleukin receptor (TIR), and tyrosine kinase.

E. Hinge domain

In some embodiments, the chimeric receptor polypeptides such as ACTR polypeptides or CAR polypeptides described herein further comprise a hinge domain that is located between the extracellular ligand-binding domain and the transmembrane domain. A hinge domain is an amino acid segment that is generally found between two domains of a protein and may allow for flexibility of the protein and movement of one or both domains relative to one another. Any amino acid sequence that provides such flexibility and movement of the extracellular ligand-binding domain relative to the transmembrane domain of the chimeric receptor polypeptide can be used.

Hinge domains of any protein known in the art to comprise a hinge domain are compatible for use in the chimeric receptor polypeptides described herein. In some embodiments, the hinge domain is at least a portion of a hinge domain of a naturally occurring protein and confers flexibility to the chimeric receptor polypeptide. In one embodiment the chimeric receptor polypeptide comprises a hinge domain, which is a hinge domain selected from the list of CD28, CD16A, CD8, IgG, murine CD8a, and DAP12. In some embodiments, the hinge domain is of CD8 (e.g., the hinge domain is of CD8α). In some embodiments, the hinge domain is a portion of the hinge domain of CD8, e.g., a fragment containing at least 15 (e.g., 20, 25, 30, 35, or 40) consecutive amino acids of the hinge domain of CD8. In some embodiments, the hinge domain is of CD28. In some embodiments, the hinge domain is a portion of CD28, e.g., a fragment containing at least 15 (e.g., 20, 25, 30, 35, or 40) consecutive amino acids of the hinge domain of CD28. The hinge domain and/or the transmembrane domain may be linked to additional amino acids (e.g., 15 aa, 10-aa, 8-aa, 6-aa, or 4-aa) at the N-terminal portion, at the C-terminal portion, or both. Examples can be found, e.g., in Ying et al., Nat Med, 25(6): 947-953 (2019).

In some embodiments, the hinge domain is of CD16A receptor, for example, the whole hinge domain of a CD16A receptor or a portion thereof, which may consist of up to 40 consecutive amino acid residues of the CD16A receptor (e.g., 20, 25, 30, 35, or 40). Such a chimeric receptor polypeptide (e.g., an ACTR polypeptide) may contain no hinge domain from a different receptor (a non-CD16A receptor). In some cases, the chimeric receptor polypeptide described herein may be free of a hinge domain from any non-CD16A receptor. In some instances, such a chimeric receptor polypeptide may be free of any hinge domain.

Hinge domains of IgG antibodies, such as an IgG, IgA, IgM, IgE, or IgD antibodies, are also compatible for use in the chimeric receptor polypeptides described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody, preferably IgG1 and IgG4. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptor polypeptides described herein. In some embodiments, the hinge domain between the C-terminus of the extracellular target-binding domain and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more. In some embodiments, the hinge domain is (Gly₄Ser)ₙ, wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In certain embodiments, n can be an integer greater than 60. In some embodiments, the hinge domain is (Gly₄Ser)₃. In some embodiments, the hinge domain is (Gly₄Ser)₆. In some embodiments, the hinge domain is (Gly₄Ser)₉. In some embodiments, the hinge domain is (Gly₄Ser)₁₂. In some embodiments, the hinge domain is (Gly₄Ser)₁₅. In some embodiments, the hinge domain is (Gly₄Ser)₃₀. In some embodiments, the hinge domain is (Gly₄Ser)₄₅. In some embodiments, the hinge domain is (Gly₄Ser)₆₀.

In other embodiments, the hinge domain is an extended recombinant polypeptide (XTEN), which is an unstructured polypeptide consisting of hydrophilic residues of varying lengths (e.g., 10-80 amino acid residues). Amino acid sequences of XTEN peptides will be evident to one of skill in the art and can be found, for example, in U.S. Pat. No. 8,673,860, the relevant disclosures of which are incorporated by reference herein. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

Any of the hinge domains used for making the chimeric receptor polypeptide as described herein may contain up to 250 amino acid residues. In some instances, the chimeric receptor polypeptide may contain a relatively long hinge domain, for example, containing 150-250 amino acid residues (e.g., 150-180 amino acid residues, 180-200 amino acid residues, or 200-250 amino acid residues). In other instances, the chimeric receptor polypeptide may contain a medium sized hinge domain, which may contain 60-150 amino acid residues (e.g., 60-80, 80-100, 100-120, or 120-150 amino acid residues). In some instances, the hinge domain may be a flexible linker consisting of glycine and serine amino acids having a length between 15 and 60 amino acids, preferably composed of Gly₄Ser units, especially one of the linkers of SEQ ID NO: 15 to SEQ ID NO: 17. Alternatively, the chimeric receptor polypeptide may contain a short hinge domain, which may contain less than 60 amino acid residues (e.g., 1-30 amino acids or 31-60 amino acids). In some embodiments, a chimeric receptor polypeptide (e.g., an ACTR polypeptide) described herein contains no hinge domain or no hinge domain from a non-CD16A receptor. The amino acid sequences of exemplary hinge domains are provided in Table 7.

TABLE 7
Exemplary Hinge Domains

| Hinge domain | Sequences | SEQ ID NO. |
|---|---|---|
| CD8α hinge | TTTPAPRPPTPAPTIASQPLSLRPEACRPAAGGAVHTR | SEQ ID NO: 4 |
| domain | GLDFAC | |
| IgG1 (hinge- | EPKSCDKTHTCPPCPAPELLGGPSVFLFPPKPKDTLMI | SEQ ID NO: 5 |
| CH2-CH3) | SRTPEVTCVVVDVSHEDPEVKFNWYVDGVEVHNAKTKP | |
| | REEQYNSTYRVVSVLTVLHQDWLNGKEYKCKVSNKALP | |
| | APIEKTISKAKGQPREPQVYTLPPSRDELTKNQVSLTC | |
| | LVKGFYPSDIAVEWESNGQPENNYKTTPPVLDSDGSFF | |
| | LYSKLTVDKSRWQQGNVFSCSVMHEALHNHYTQKSLSL | |
| | SPGK | |
| IgG1 (hinge- | EPKSCDKTHTCPGQPREPQVYTLPPSRDELTKNQVSLT | SEQ ID NO: 6 |
| CH3) | CLVKGFYPSDIAVEWESNGQPENNYKTTPPVLDSDGSF | |
| | FLYSKLTVDKSRWQQGNVFSCSVMHEALHNHYTQKSLS | |
| | LSPGK | |
| IgG1 hinge | EPKSCDKTHTCP | SEQ ID NO: 7 |
| domain | | |
| IgG4 hinge | ESKYGPPCPPCP | SEQ ID NO: 8 |
| domain | | |
| CD8α-Fragment | TTTPAPRPPTPAPTIASQPLSLRPEAFAC | SEQ ID NO: 9 |
| 1 (30aa) | | |
| CD8α-Fragment | TTTPAPRPPTPFAC | SEQ ID NO: 10 |
| 2 (15aa) | | |
| CD28 | IEVMYPPPYLDNEKSNGTIIHVKGKHLCPSPLFPGPSK | SEQ ID NO: 11 |
| | P | |
| CD28 (39 aa) | IEVMYPPPYLDNEKSNGTIIHVKGKHLCPSPLFPGPSK | SEQ ID NO: 12 |
| | P | |
| CD28 (26 aa) | KSNGTIIHVKGKHLCPSPLFPGPSKP | SEQ ID NO: 13 |
| CD28 (16 aa) | GKHLCPSPLFPGPSKP | SEQ ID NO: 14 |
| (Gly₄Ser)ₓ₆ | GGGGSGGGGSGGGGSGGGGSGGGGSGGGGS | SEQ ID NO: 15 |
| (30aa) | | |
| (Gly₄Ser)ₓ₉ | GGGGSGGGGSGGGGSGGGGSGGGGSGGGGSGGGGSGGG | SEQ ID NO: 16 |
| (45aa) | GSGGGGS | |
| (Gly₄Ser)ₓ₁₂ | GGGGSGGGGSGGGGSGGGGSGGGGSGGGGSGGGGSGGG | SEQ ID NO: 17 |
| (60aa) | GSGGGGSGGGGSGGGGSGGGGS | |

### F. Signal Peptide

In some embodiments, the chimeric receptor polypeptide (e.g., ACTR polypeptide or CAR polypeptide) may also comprise a signal peptide (also known as a signal sequence) at the N-terminus of the polypeptide. In general, signal sequences are peptide sequences that target a polypeptide to the desired site in a cell. In some embodiments, the signal sequence targets the chimeric receptor polypeptide to the secretory pathway of the cell and will allow for integration and anchoring of the chimeric receptor polypeptide into the lipid bilayer. Signal sequences including signal sequences of naturally occurring proteins or synthetic, non-naturally occurring signal sequences that are compatible for use in the chimeric receptor polypeptides described herein will be evident to one of skill in the art. In some embodiments, the signal sequence from CD8α (SEQ ID NO: 1). In some embodiments, the signal sequence is from CD28 (SEQ ID NO: 2). In other embodiments, the signal sequence is from the murine kappa chain. In yet other embodiments, the signal sequence is from CD16. See Table 8 below.

TABLE 8
Exemplary Signal Peptides

| Signal | | |
|---|---|---|
| Peptide | Sequences | SEQ ID NO. |
| CD8a | MALPVTALLLPLALLLHAARP | SEQ ID NO: 1 |
| CD28 | MLRLLLALNLFPSIQVTG | SEQ ID NO: 2 |
| Murine | METDTLLLWVLLLWVPGSTG | SEQ ID NO: 3 |
| k chain | | |

In some instances, any of the chimeric receptor polypeptides disclosed herein may further comprise a protein tag, examples of which are provided in Table 9 below.

TABLE 9
Exemplary Protein Tags

| Protein Tag | Sequences | SEQ ID NO. |
|---|---|---|
| 2x V5 Tag | GKPIPNPLLGLDSTGK | SEQ ID NO: 62 |
| | PIPNPLLGLDST | |
| 6xHis Tag | HHHHHH | SEQ ID NO: 63 |
| 2xFlag Tag | DYKDDDDKDYKDDDDK | SEQ ID NO: 64 |
| 3xHA Tag | YPYDVPDYAYPYDVPD | SEQ ID NO: 65 |
| | YAYPYDVPDYA | |

### G. Examples of ACTR Polypeptides

Exemplary ACTR constructs for use with the methods and compositions described herein may be found, for example, in the instant description and figures or may be found in WO2016/040441A1, WO2017/161333, and WO2018/140960, each of which is incorporated by reference herein for this purpose. The ACTR polypeptides described herein may comprise a CD16A extracellular domain with binding affinity and specificity for the Fc portion of an IgG molecule, a transmembrane domain, and a CD3ζ cytoplasmic signaling domain. In some embodiments, the ACTR polypeptides may further include one or more co-stimulatory signaling domains, one of which may be a CD28 co-stimulatory signaling domain or a 4-1BB co-stimulatory signaling domain. The ACTR polypeptides are configured such that, when expressed on a host cell, the extracellular ligand-binding domain is located extracellularly for binding to a target molecule and the CD3ζ cytoplasmic signaling domain. The co-stimulatory signaling domain may be located in the cytoplasm for triggering activation and/or effector signaling.

In some embodiments, an ACTR polypeptide as described herein may comprise, from N-terminus to C-terminus, the Fc binding domain such as a CD16A extracellular domain, the transmembrane domain, the optional one or more co-stimulatory domains (e.g., a CD28 co-stimulatory domain, a 4-1BB co-stimulatory signaling domain, an OX40 co-stimulatory signaling domain, a CD27 co-stimulatory signaling domain, or an ICOS co-stimulatory signaling domain), and the CD3ζ cytoplasmic signaling domain.

Alternatively or in addition, the ACTR polypeptides described herein may contain two or more co-stimulatory signaling domains, which may link to each other or be separated by the cytoplasmic signaling domain. The extracellular Fc binder, transmembrane domain, optional co-stimulatory signaling domain(s), and cytoplasmic signaling domain in an ACTR polypeptide may be linked to each other directly, or via a peptide linker. In some embodiments, any of the ACTR polypeptides described herein may comprise a signal sequence at the N-terminus.

Table 10 provides exemplary ACTR polypeptides described herein. These exemplary constructs have, from N-terminus to C-terminus in order, the signal sequence, the Fc binding domain (e.g., an extracellular domain of an Fc receptor), the hinge domain, and the transmembrane, while the positions of the optional co-stimulatory domain and the cytoplasmic signaling domain can be switched.

TABLE 10
Exemplary Components of ACTR Polypeptides.

| | | Extracellular | | | | |
|---|---|---|---|---|---|---|
| | | domain | Hinge | | Co- | Cytoplasmic |
| | Signal | of Fc | domain | Transmembrane | stimulatory | Signaling |
| # | Sequence | receptor (a) | (e) | domain (b) | domain (d) | domain (c) |
| 1 | CD8α | CD16A- | CD8α | CD8α | 4-1BB | CD3ζ |
| | | V158 | | | | |
| 2 | CD8α | CD16A- | CD8α | 4-1BB | 4-1BB | CD3ζ |
| | | V158 | | | | |
| 3 | CD8α | CD16A- | CD8α | CD28 | 4-1BB | CD3ζ |
| | | V158 | | | | |
| 4 | CD8α | CD16A- | CD8α | CD34 | 4-1BB | CD3ζ |
| | | V158 | | | | |
| 5 | CD8α | CD16A- | CD8α | Designed | 4-1BB | CD3ζ |
| | | V158 | | hydrophobic | | |
| 6 | CD8α | CD32A | CD8α | CD8α | 4-1BB | CD3ζ |
| 7 | CD8α | CD16A- | CD8α | CD8α | CD28 | CD3ζ |
| | | V158 | | | | |
| 8 | CD8α | CD16A- | CD8α | CD8α | OX40 | CD3ζ |
| | | V158 | | | | |
| 9 | CD8α | CD16A- | CD8α | CD8α | CD28 + | CD3ζ |
| | | V158 | | | 4-1BB | |
| 10 | CD8α | CD16A- | None | CD8α | 4-1BB | CD3ζ |
| | | V158 | | | | |
| 11 | CD8α | CD16A- | XTEN | CD8α | 4-1BB | CD3ζ |
| | | V158 | | | | |
| 12 | CD8α | CD16A- | CD8α | CD8α | CD28 LL to | CD3ζ |
| | | V158 | | | GG mutant | |
| 13 | CD8α | CD16A- | CD8α | CD8α | CD28 LL to | CD3ζ |
| | | V158 | | | GG mutant + | |
| | | | | | 4-1BB | |
| 14 | CD8α | CD16A- | CD8α | CD4 | 4-1BB | CD3ζ |
| | | V158 | | | | |
| 15 | CD8α | CD16A- | CD8α | CD4 | CD28 LL to | CD3ζ |
| | | V158 | | | GG mutant + | |
| | | | | | 4-1BB | |
| 16 | CD8α | CD16A- | CD8α | FcεRIγ | 4-1BB | CD3ζ |
| | | V158 | | | | |
| 17 | CD8α | CD16A- | CD8α | designed | 4-1BB | CD3ζ |
| | | V158 | | hydrophobic, | | |
| | | | | predicted | | |
| | | | | dimerization | | |
| 18 | CD8α | CD16A- | CD8α | CD8β | 4-1BB | CD3ζ |
| | | V158 | | | | |
| 19 | CD8α | CD16A- | CD8α | C16α | 4-1BB | CD3ζ |
| | | V158 | | | | |
| 20 | CD8α | CD16A- | CD8α | OX40 | 4-1BB | CD3ζ |
| | | V158 | | | | |
| 21 | CD8α | CD16A- | CD8α | CD3ζ | 4-1BB | CD3ζ |
| | | V158 | | | | |
| 22 | CD8α | CD16A- | CD8α | CD3ε | 4-1BB | CD3ζ |
| | | V158 | | | | |
| 23 | CD8α | CD16A- | CD8α | CD3γ | 4-1BB | CD3ζ |
| | | V158 | | | | |
| 24 | CD8α | CD16A- | CD8α | CD3δ | 4-1BB | CD3ζ |
| | | V158 | | | | |
| 25 | CD8α | CD16A- | CD8α | TCR-α | 4-1BB | CD3ζ |
| | | V158 | | | | |
| 26 | CD8α | CD16A- | CD8α | CD32 | 4-1BB | CD3ζ |
| | | V158 | | | | |
| 27 | CD8α | CD16A- | CD8α | CD64 | 4-1BB | CD3ζ |
| | | V158 | | | | |
| 28 | CD8α | CD16A- | CD8α | VEGFR2 | 4-1BB | CD3ζ |
| | | V158 | | | | |
| 29 | CD8α | CD16A- | CD8α | FAS | 4-1BB | CD3ζ |
| | | V158 | | | | |
| 30 | CD8α | CD16A- | CD8α | FGFR2B | 4-1BB | CD3ζ |
| | | V158 | | | | |
| 31 | CD8α | CD16A- | CD8α | CD8α | 4-1BB | CD3? |
| | | F158 | | | | |
| 32 | CD8α | CD64A | CD8α | CD8α | 4-1BB | CD3ζ |
| 33 | CD8α | CD16A- | IgG1 (hinge- | CD8α | 4-1BB | CD3ζ |
| | | V158 | CH2-CH3) | | | |
| 34 | CD8α | CD16A- | IgG1 (hinge- | CD8α | 4-1BB | CD3ζ |
| | | V158 | CH3) | | | |
| 35 | CD8α | CD16A- | IgG1 (hinge) | CD8α | 4-1BB | CD3ζ |
| | | V158 | | | | |
| 36 | CD8α | CD16A- | CD8α | CD8α | 4-1BB | CD3ζ |
| | | V158 | fragment 1 | | | |
| | | | (30 aa) | | | |
| 37 | CD8α | CD16A- | CD8α | CD8α | 4-1BB | CD3ζ |
| | | V158 | fragment 2 | | | |
| | | | (15 aa) | | | |
| 38 | CD8α | CD16A- | (Gly₄Ser)₃ | CD8α | 4-1BB | CD3ζ |
| | | V158 | | | | |
| 39 | CD8α | CD16A- | (Gly₄Ser)₆ | CD8α | 4-1BB | CD3c |
| | | V158 | | | | |
| 40 | CD8α | CD16A- | (Gly₄Ser)₉ | CD8α | 4-1BB | CD3ζ |
| | | V158 | | | | |
| 41 | CD8α | CD16A- | (Gly₄Ser)₁₂ | CD8α | 4-1BB | CD3ζ |
| | | V158 | | | | |
| 42 | CD8α | CD16A- | XTEN | CD8α | 4-1BB | CD3ζ |
| | | V158 | (60 aa) | | | |
| 43 | CD8α | CD16A- | XTEN | CD8α | 4-1BB | CD3ζ |
| | | V158 | (30 aa) | | | |
| 44 | CD8α | CD16A- | XTEN | CD8α | 4-1BB | CD3ζ |
| | | V158 | (15 aa) | | | |
| 45 | CD28 | CD16A- | CD8α | CD8α | 4-1BB | CD3ζ |
| | | V158 | | | | |
| 46 | Murine κ | CD16A- | CD8α | CD8α | 4-1BB | CD3ζ |
| | chain | V158 | | | | |
| 47 | CD16 | CD16A- | CD8α | CD8α | 4-1BB | CD3ζ |
| | | V158 | | | | |
| 48 | CD8α | CD16A- | CD8α | CD8α | ICOS | CD3ζ |
| | | V158 | | | | |
| 49 | CD8α | CD16A- | CD8α | CD8α | CD27 | CD3ζ |
| | | V158 | | | | |
| 50 | CD8α | CD16A- | CD8α | CD8α | GITR | CD3ζ |
| | | V158 | | | | |
| 51 | CD8α | CD16A- | CD8α | CD8α | HVEM | CD3 |
| | | V158 | | | | |
| 52 | CD8α | CD16A- | CD8α | CD8α | TIM1 | CD3ζ |
| | | V158 | | | | |
| 53 | CD8α | CD16A- | CD8α | CD8α | LFA1 | CD3ζ |
| | | V158 | | | (CD11a) | |
| 54 | CD8α | CD16A- | CD8α | CD8α | CD2 | CD3ζ |
| | | V158 | | | | |
| 55 | CD8α | CD16A- | CD8α | FcεR1γ | 4-1BB | FcεR1γ |
| | | V158 | | | | |
| 56 | CD8α | CD16A- | CD8α | CD8α | 4-1BB | FcεR1γ |
| | | V158 | | | | |
| 57 | CD8α | CD16A- | CD28 | CD28 | CD28 | CD3ζ |
| | | V158 | (e.g., 39 aa) | | | |
| 58 | CD8α | CD16A- | none | CD8 | CD28 | CD3ζ |
| | | V158 | | | | |
| 59 | CD8α | CD16A- | CD8 | CD8 | CD28 + | CD3ζ |
| | | V158 | | | CD27 | |
| 60 | CD8α | CD16A- | CD8 | CD8 | CD28 + | CD3ζ |
| | | V158 | | | OX40 | |
| 61 | CD8α | CD16A- | CD8 | CD8 | 4-1BB + | CD3ζ |
| | | V158 | | | CD28 | |
| 62 | CD8α | CD16A- | CD28 | CD28 | CD28 + | CD3c |
| | | V158 | | | 4-1BB | |
| 63 | CD8α | CD16A- | CD28 | CD28 | 4-1BB | CD3ζ |
| | | V158 | | | | |
| 64 | CD8α | CD16A- | CD8 | CD8 | CD27 | CD3ζ |
| | | V158 | | | | |
| 65 | CD8α | CD16A- | CD8 | CD8 | CD28 | CD3ζ |
| | | V158 | | | | |
| 66 | CD8α | CD16A- | CD8 | CD8 | ICOS | CD3ζ |
| | | V158 | | | | |
| 67 | CD8α | CD16A- | CD8 | CD8 | OX40 | CD3ζ |
| | | V158 | | | | |
| 68 | CD8α | CD16A- | CD8 | CD8 | CD28 and | CD3ζ |
| | | V158 | | | ICOS | |
| 69 | CD8α | CD16A- | none | CD8 | 4-1BB | CD3ζ |
| | | V158 | | | | |
| 70 | CD8α | CD16A- | none | CD8 | CD27 | CD3ζ |
| | | V158 | | | | |
| 71 | CD8α | CD16A- | none | CD8 | ICOS | CD3ζ |
| | | V158 | | | | |
| 72 | CD8α | CD16A- | none | CD8 | OX40 | CD3? |
| | | V158 | | | | |
| 73 | CD8α | CD16A- | none | CD8 + 4 aa | 4-1BB | CD3ζ |
| | | V158 | | | | |
| 74 | CD8α | CD16A- | none | CD8 + 4 aa | CD28 | CD3ζ |
| | | V158 | | | | |
| 75 | CD8α | CD16A- | CD8 | CD28 | CD28 | CD3? |
| | | V158 | | | | |
| 76 | CD8α | CD16A- | CD28 | CD28 | CD28 | CD3ζ |
| | | V158 | (26 aa) | | | |
| 77 | CD8α | CD16A- | CD28 | CD28 | CD28 | CD3ζ |
| | | V158 | (16 aa) | | | |
| 78 | CD8α | CD16A- | none | CD28 | CD28 | CD3ζ |
| | | V158 | | | | |
| 79 | CD8α | CD16A- | CD8 | CD8 | 41BB | CD3ζ |
| | | V158 | | | | |
| 80 | CD8α | CD16A- | CD28 (39 | CD8 | CD28 | CD3ζ |
| | | V158 | aa) | | | |

### H. Examples of CAR Polypeptides

Exemplary CAR polypeptides for use with the methods and compositions described herein may be found, for example, in the instant description and figures or as those known in the art. The CAR polypeptides described herein may comprise an extracellular domain comprising a single-chain antibody fragment (scFv) with binding affinity and specificity for an antigen of interest (e.g., those listed in Table 4, a transmembrane domain (e.g, those listed in Table 5), preferably a CD8a transmembrane domain), a co-stimulatory domain (e.g., those listed in Table 6) and a CD3ζ cytoplasmic signaling domain. In some embodiments, the CAR polypeptide may further comprise a hinge domain (e.g., those listed in Table 7).

In specific examples, a CAR polypeptide described herein may comprise (i) a CD28 co-stimulatory domain or a 4-1BB co-stimulatory domain; and (ii) a CD28 transmembrane domain, a CD28 hinge domain, or a combination thereof. In further specific examples, a CAR polypeptide described herein may comprise (i) a CD28 co-stimulatory domain or a 4-1BB co-stimulatory domain, (ii) a CD8α transmembrane domain, a CD8α hinge domain, or a combination thereof. In some embodiments, the CAR polypeptides may further include one or more co-stimulatory signaling domains, one of which may be a CD28 co-stimulatory signaling domain or a 4-1BB co-stimulatory signaling domain. In other examples, a CAR polypeptide described herein may comprise (i) a CD28 co-stimulatory domain or a 4-1BB co-stimulatory domain, (ii) a CD28 transmembrane domain, a CD8α hinge domain, or a combination thereof.

In an exemplary embodiment, the CAR polypeptide comprises (i) a CD8α hinge domain (ii) a CD8α transmembrane domain, (iii) a CD28 co-stimulatory domain or a 4-1BB co-stimulatory domain, (iv) a CD3ζ cytoplasmic signaling domain or a combination thereof. In other embodiments, the CAR polypeptide comprising two co-stimulatory domains further comprises (i) a CD8α or CD28 hinge domain (ii) a CD8α or CD28 transmembrane domain, (iii) a CD28 co-stimulatory domain or a 4-1BB co-stimulatory domain, (iv) a OX40L co-stimulatory domain or a 2B4 co-stimulatory domain or a DAP10 co-stimulatory domain or a DNAM-1 co-stimulatory domain or a NKG2D co-stimulatory domain or a NKp30 co-stimulatory domain or a NKp44 co-stimulatory domain or a NKp46 co-stimulatory domain or a JAMAL co-stimulatory domain, (v) a CD3ζ cytoplasmic signaling domain or a combination thereof. In another exemplary embodiment, the CAR polypeptide comprising two co-stimulatory domains further comprises (i) a CD8α hinge domain (ii) a CD28 transmembrane domain, (iii) a CD28 co-stimulatory domain or a 4-1BB co-stimulatory domain, (iv) a OX40L co-stimulatory domain or a 2B4 co-stimulatory domain or a DAP10 co-stimulatory domain or a DNAM-1 co-stimulatory or JAMAL co-stimulatory domain, (v) a CD3ζ cytoplasmic signaling domain or a combination thereof.

In another exemplary embodiment, the CAR polypeptide comprising two co-stimulatory domains further comprises (i) a CD8α hinge domain (ii) a CD28 transmembrane domain or a NKp44 transmembrane domain or a NKG2D transmembrane domain or a NKp46 transmembrane domain, (iii) a CD28 co-stimulatory domain or a 4-1BB co-stimulatory domain or a 2B4 co-stimulatory domain or a DAP10 co-stimulatory, (iv) an OX40L co-stimulatory domain or a 2B4 co-stimulatory domain or a DAP10 co-stimulatory domain or a DAP12 or a DNAM-1 co-stimulatory or JAMAL co-stimulatory domain, (v) a CD3ζ cytoplasmic signaling domain or DAP12 cytoplasmic signaling domain or 2B4 cytoplasmic signaling domain or a combination thereof. In an exemplary embodiment, the CAR polypeptide comprises (i) a CD8α hinge domain (ii) a CD28 transmembrane domain, (iii) a CD28 co-stimulatory domain or a 4-1BB co-stimulatory domain, (iv) an OX40L co-stimulatory domain or an OX40 co-stimulatory domain, (v) a CD3ζ cytoplasmic signaling domain or a combination thereof.

For example, the CAR polypeptide may comprise an amino acid sequence selected from SEQ ID NO: 78 or SEQ ID NO: 79 provided below.

The CAR polypeptides are configured such that, when expressed on a host cell (e.g., T or NK cell), the extracellular antigen-binding domain is located extracellularly for binding to a target molecule (e.g., a tumor antigen) and the CD3ζ cytoplasmic signaling domain. The co-stimulatory signaling domain may be located in the cytoplasm for triggering activation and/or effector signaling.

In some embodiments, a CAR polypeptide as described herein may comprise, from N-terminus to C-terminus, the extracellular antigen binding domain, the transmembrane domain, the optional one or more co-stimulatory domains (e.g., a CD28 co-stimulatory domain, a 4-1BB co-stimulatory signaling domain, an OX40L co-stimulatory signaling domain an OX40 co-stimulatory signaling domain, a CD27 co-stimulatory signaling domain, a 2B4 co-stimulatory signaling domain or an ICOS co-stimulatory signaling domain), and the CD3ζ cytoplasmic signaling domain.

Alternatively or in addition, the CAR polypeptides described herein may contain two or more co-stimulatory signaling domains, which may link to each other or be separated by the cytoplasmic signaling domain. The extracellular antigen binding domain, transmembrane domain, optional co-stimulatory signaling domain(s), and cytoplasmic signaling domain in a CAR polypeptide may be linked to each other directly, or via a peptide linker. In some embodiments, any of the CAR polypeptides described herein may comprise a signal sequence at the N-terminus.

Tables 11-13 provide exemplary CAR polypeptides for CAR-αβ T cells, CAR-NK cells and CAR-γδ T cells described herein. These exemplary constructs have, from N-terminus to C-terminus in order, the signal sequence, the antigen binding domain (e.g., an scFv fragment targeting an antigen such as a tumor antigen or a pathogenic antigen), the hinge domain, and the transmembrane, while the positions of the optional co-stimulatory domain(s) and the cytoplasmic signaling domain can be switched.

TABLE 11
Exemplary CAR Constructs for Expression in αβ T Cells

| | | Antigen | Hinge | Transmembrane | Co-stim. | Co-stim. | Cytoplasmic |
|---|---|---|---|---|---|---|---|
| CAR | Signal | binding | domain | domain | domain | domain | signaling |
| # | Sequence | domain | (e) | (b) | (d1) | (d2) | domain (c) |
| 1 | CD8α | scFv | CD8α | CD8α | 4-1BB | none | CD3ζ |
| 2 | CD8α | scFv | CD8α | CD28 | 4-1BB | none | CD3ζ |
| 3 | CD8α | scFv | CD8α | 4-1BB | 4-1BB | none | CD3ζ |
| 4 | CD8α | scFv | CD8α | CD4 | 4-1BB | none | CD3ζ |
| 5 | CD8α | scFv | CD8α | FcεRIγ | 4-1BB | none | CD3ζ |
| 6 | CD8α | scFv | CD8α | CD8β | 4-1BB | none | CD3ζ |
| 7 | CD8α | scFv | CD8α | C16α | 4-1BB | none | CD3ζ |
| 8 | CD8α | scFv | CD8α | OX40 | 4-1BB | none | CD3ζ |
| 9 | CD8α | scFv | CD8α | CD3ζ | 4-1BB | none | CD3ζ |
| 10 | CD8α | scFv | CD8α | CD3δ | 4-1BB | none | CD3ζ |
| 11 | CD8α | scFv | CD8α | CD3γ | 4-1BB | none | CD3ζ |
| 12 | CD8α | scFv | CD8α | CD3δ | 4-1BB | none | CD3ζ |
| 13 | CD8α | scFv | CD8α | TCR-α | 4-1BB | none | CD3ζ |
| 14 | CD8α | scFv | CD8α | CD32 | 4-1BB | none | CD3ζ |
| 15 | CD8α | scFv | CD8α | CD64 | 4-1BB | none | CD3ζ |
| 16 | CD8α | scFv | CD8α | VEGFR2 | 4-1BB | none | CD3ζ |
| 17 | CD8α | scFv | CD8α | FAS | 4-1BB | none | CD3ζ |
| 18 | CD8α | scFv | CD8α | FGFR2B | 4-1BB | none | CD3ζ |
| 19 | CD8α | scFv | CD8α | CD8α | CD28 | none | CD3ζ |
| 20 | CD8α | scFv | CD8α | CD8α | 4-1BB | none | CD3ζ |
| 21 | CD8α | scFv | CD8α | CD8α | ICOS | none | CD3ζ |
| 22 | CD8α | scFv | CD8α | CD8α | CD27 | none | CD3ζ |
| 23 | CD8α | scFv | CD8α | CD8α | GITR | none | CD3ζ |
| 24 | CD8α | scFv | CD8α | CD8α | HVEM | none | CD3ζ |
| 25 | CD8α | scFv | CD8α | CD8α | TIM1 | none | CD3ζ |
| 26 | CD8α | scFv | CD8α | CD8α | LFA1 | none | CD3ζ |
| 27 | CD8α | scFv | CD8α | CD8α | CD2 | none | CD3ζ |
| 28 | CD8α | scFv | CD8α | CD8α | OX40 | none | CD3ζ |
| 29 | CD8α | scFv | CD8α | CD8α | OX40L | none | CD3ζ |
| 30 | CD8α | scFv | CD8α | CD28 | CD28 | none | CD3ζ |
| 31 | CD8α | scFv | CD8α | CD28 | 4-1BB | none | CD3ζ |
| 32 | CD8α | scFv | CD8α | CD28 | ICOS | none | CD3ζ |
| 33 | CD8α | scFv | CD8α | CD28 | CD27 | none | CD3ζ |
| 34 | CD8α | scFv | CD8α | CD28 | GITR | none | CD3ζ |
| 35 | CD8α | scFv | CD8α | CD28 | HVEM | none | CD3ζ |
| 36 | CD8α | scFv | CD8α | CD28 | TIM1 | none | CD3ζ |
| 37 | CD8α | scFv | CD8α | CD28 | LFA1 | none | CD3ζ |
| 38 | CD8α | scFv | CD8α | CD28 | CD2 | none | CD3ζ |
| 39 | CD8α | scFv | CD8α | CD28 | OX40 | none | CD3ζ |
| 40 | CD8α | scFv | CD8α | CD28 | OX40L | none | CD3ζ |
| 41 | CD8α | scFv | CD8α | 4-1BB | CD28 | none | CD3ζ |
| 42 | CD8α | scFv | CD8α | 4-1BB | 4-1BB | none | CD3ζ |
| 43 | CD8α | scFv | CD8α | 4-1BB | ICOS | none | CD3ζ |
| 44 | CD8α | scFv | CD8α | 4-1BB | CD27 | none | CD3ζ |
| 45 | CD8α | scFv | CD8α | 4-1BB | GITR | none | CD3ζ |
| 46 | CD8α | scFv | CD8α | 4-1BB | HVEM | none | CD3ζ |
| 47 | CD8α | scFv | CD8α | 4-1BB | TIM1 | none | CD3ζ |
| 48 | CD8α | scFv | CD8α | 4-1BB | LFA1 | none | CD3ζ |
| 49 | CD8α | scFv | CD8α | 4-1BB | CD2 | none | CD3ζ |
| 50 | CD8α | scFv | CD8α | 4-1BB | OX40 | none | CD3ζ |
| 51 | CD8α | scFv | CD8α | 4-1BB | OX40L | none | CD3ζ |
| 52 | CD8α | scFv | CD8α | CD8α | CD28 | 4-1BB | CD3ζ |
| 53 | CD8α | scFv | CD8α | CD8α | CD28 | CD27 | CD3ζ |
| 54 | CD8α | scFv | CD8α | CD8α | CD28 | OX40 | CD3ζ |
| 55 | CD8α | scFv | CD8α | CD8α | CD28 | ICOS | CD3ζ |
| 56 | CD8α | scFv | CD8α | CD8α | CD28 | OX40L | CD3ζ |
| 57 | CD8α | scFv | CD8α | CD8α | 4-1BB | CD27 | CD3ζ |
| 58 | CD8α | scFv | CD8α | CD8α | 4-1BB | OX40 | CD3ζ |
| 59 | CD8α | scFv | CD8α | CD8α | 4-1BB | ICOS | CD3ζ |
| 60 | CD8α | scFv | CD8α | CD8α | 4-1BB | OX40L | CD3ζ |
| 61 | CD8α | scFv | CD8α | CD28 | CD28 | 4-1BB | CD3ζ |
| 62 | CD8α | scFv | CD8α | CD28 | CD28 | CD27 | CD3ζ |
| 63 | CD8α | scFv | CD8α | CD28 | CD28 | OX40 | CD3ζ |
| 64 | CD8α | scFv | CD8α | CD28 | CD28 | ICOS | CD3ζ |
| 65 | CD8α | scFv | CD8α | CD28 | CD28 | OX40L | CD3ζ |
| 66 | CD8α | scFv | CD8α | CD28 | 4-1BB | CD27 | CD3ζ |
| 67 | CD8α | scFv | CD8α | CD28 | 4-1BB | OX40 | CD3ζ |
| 68 | CD8α | scFv | CD8α | CD28 | 4-1BB | ICOS | CD3ζ |
| 69 | CD8α | scFv | CD8α | CD28 | 4-1BB | OX40L | CD3ζ |
| 70 | CD8α | scFv | CD8α | 4-1BB | CD28 | 4-1BB | CD3ζ |
| 71 | CD8α | scFv | CD8α | 4-1BB | CD28 | CD27 | CD3ζ |
| 72 | CD8α | scFv | CD8α | 4-1BB | CD28 | OX40 | CD3ζ |
| 73 | CD8α | scFv | CD8α | 4-1BB | CD28 | ICOS | CD3ζ |
| 74 | CD8α | scFv | CD8α | 4-1BB | CD32 | OX40L | CD3ζ |
| 75 | CD8α | scFv | CD8α | 4-1BB | 4-1BB | CD27 | CD3ζ |
| 76 | CD8α | scFv | CD8α | 4-1BB | 4-1BB | OX40 | CD3ζ |
| 77 | CD8α | scFv | CD8α | 4-1BB | 4-1BB | ICOS | CD3ζ |
| 78 | CD8α | scFv | CD8α | 4-1BB | 4-1BB | OX40L | CD3ζ |

TABLE 12
Exemplary CAR Constructs for Expressin in NK Cells

| | | Antigen | Hinge | | Co-stim. | Co-stim. | Cytoplasmic |
|---|---|---|---|---|---|---|---|
| | Signal | binding | domain | Transmembrane | domain | domain | Signaling |
| CAR # | Sequence | domain | (e) | domain (b) | (d1) | (d2) | domain (c) |
| 1 | CD8α | scFv | CD8α | CD8α | 4-1BB | none | CD3ζ |
| 2 | CD8α | scFv | CD8α | CD8β | 4-1BB | none | CD3ζ |
| 3 | CD8α | scFv | CD8α | CD28 | 4-1BB | none | CD3ζ |
| 4 | CD8α | scFv | CD8α | 4-1BB | 4-1BB | none | CD3ζ |
| 5 | CD8α | scFv | CD8α | CD4 | 4-1BB | none | CD3ζ |
| 6 | CD8α | scFv | CD8α | FcεRIγ | 4-1BB | none | CD3ζ |
| 7 | CD8α | scFv | CD8α | CD8β | 4-1BB | none | CD3ζ |
| 8 | CD8α | scFv | CD8α | C16α | 4-1BB | none | CD3ζ |
| 9 | CD8α | scFv | CD8α | OX40 | 4-1BB | none | CD3ζ |
| 10 | CD8α | scFv | CD8α | CD3ζ | 4-1BB | none | CD3ζ |
| 11 | CD8α | scFv | CD8α | CD3ε | 4-1BB | none | CD3ζ |
| 12 | CD8α | scFv | CD8α | CD3γ | 4-1BB | none | CD3ζ |
| 13 | CD8α | scFv | CD8α | CD3δ | 4-1BB | none | CD3ζ |
| 14 | CD8α | scFv | CD8α | TCR-α | 4-1BB | none | CD3ζ |
| 15 | CD8α | scFv | CD8α | CD32 | 4-1BB | none | CD3ζ |
| 16 | CD8α | scFv | CD8α | CD64 | 4-1BB | none | CD3ζ |
| 17 | CD8α | scFv | CD8α | VEGFR2 | 4-1BB | none | CD3ζ |
| 18 | CD8α | scFv | CD8α | FAS | 4-1BB | none | CD3ζ |
| 19 | CD8α | scFv | CD8α | FGFR2B | 4-1BB | none | CD3ζ |
| 20 | CD8α | scFv | CD8α | NKG2D | 4-1BB | none | CD3ζ |
| 21 | CD8α | scFv | CD8α | NKp44 | 4-1BB | none | CD3ζ |
| 22 | CD8α | scFv | CD8α | NKp46 | 4-1BB | none | CD3ζ |
| 23 | CD8α | scFv | CD8α | CD8α | CD28 | none | CD3ζ |
| 24 | CD8α | scFv | CD8α | CD8α | 4-1BB | none | CD3ζ |
| 25 | CD8α | scFv | CD8α | CD8α | DAP10 | none | CD3ζ |
| 26 | CD8α | scFv | CD8α | CD8α | DAP12 | none | CD3ζ |
| 27 | CD8α | scFv | CD8α | CD8α | 2B4 | none | CD3ζ |
| 28 | CD8α | scFv | CD8α | CD8α | CD27 | none | CD3ζ |
| 29 | CD8α | scFv | CD8α | CD8α | CD2 | none | CD3ζ |
| 30 | CD8α | scFv | CD8α | CD8α | OX40 | none | CD3ζ |
| 31 | CD8α | scFv | CD8α | CD8α | OX40L | none | CD3ζ |
| 32 | CD8α | scFv | CD8α | CD8α | DNAM-1 | none | CD3ζ |
| 33 | CD8α | scFv | CD8α | CD8α | NKp30 | none | CD3ζ |
| 34 | CD8α | scFv | CD8α | CD8α | NKp44 | none | CD3ζ |
| 35 | CD8α | scFv | CD8α | CD8α | NKp46 | none | CD3ζ |
| 36 | CD8α | scFv | CD8α | CD8α | ICOS | none | CD3ζ |
| 37 | CD8α | scFv | CD8α | CD8α | NKG2D | none | CD3ζ |
| 38 | CD8α | scFv | CD8α | CD8α | JAMAL | none | CD3ζ |
| 39 | CD8α | scFv | CD8α | CD8α | GITR | none | CD3ζ |
| 40 | CD8α | scFv | CD8α | CD8α | HVEM | none | CD3ζ |
| 41 | CD8α | scFv | CD8α | CD8α | TIM1 | none | CD3ζ |
| 42 | CD8α | scFv | CD8α | CD8α | LFA1 | none | CD3ζ |
| 43 | CD8α | scFv | CD8α | CD28 | CD28 | none | CD3ζ |
| 44 | CD8α | scFv | CD8α | CD28 | 4-1BB | none | CD3ζ |
| 45 | CD8α | scFv | CD8α | CD28 | DAP10 | none | CD3ζ |
| 46 | CD8α | scFv | CD8α | CD28 | DAP12 | none | CD3ζ |
| 47 | CD8α | scFv | CD8α | CD28 | 2B4 | none | CD3ζ |
| 48 | CD8α | scFv | CD8α | CD28 | CD27 | none | CD3ζ |
| 49 | CD8α | scFv | CD8α | CD28 | CD2 | none | CD3ζ |
| 50 | CD8α | scFv | CD8α | CD28 | OX40 | none | CD3ζ |
| 51 | CD8α | scFv | CD8α | CD28 | OX40L | none | CD3ζ |
| 52 | CD8α | scFv | CD8α | CD28 | DNAM-1 | none | CD3ζ |
| 53 | CD8α | scFv | CD8α | CD28 | NKp30 | none | CD3ζ |
| 54 | CD8α | scFv | CD8α | CD28 | NKp44 | none | CD3ζ |
| 55 | CD8α | scFv | CD8α | CD28 | NKp46 | none | CD3ζ |
| 56 | CD8α | scFv | CD8α | CD28 | ICOS | none | CD3ζ |
| 57 | CD8α | scFv | CD8α | CD28 | NKG2D | none | CD3ζ |
| 58 | CD8α | scFv | CD8α | CD28 | JAMAL | none | CD3ζ |
| 59 | CD8α | scFv | CD8α | CD28 | GITR | none | CD3ζ |
| 60 | CD8α | scFv | CD8α | CD28 | HVEM | none | CD3ζ |
| 61 | CD8α | scFv | CD8α | CD28 | TIM1 | none | CD3ζ |
| 62 | CD8α | scFv | CD8α | CD28 | LFA1 | none | CD3ζ |
| 63 | CD8α | scFv | CD8α | 4-1BB | CD28 | none | CD3ζ |
| 64 | CD8α | scFv | CD8α | 4-1BB | 4-1BB | none | CD3ζ |
| 65 | CD8α | scFv | CD8α | 4-1BB | DAP10 | none | CD3ζ |
| 66 | CD8α | scFv | CD8α | 4-1BB | DAP12 | none | CD3ζ |
| 67 | CD8α | scFv | CD8α | 4-1BB | 2B4 | none | CD3ζ |
| 68 | CD8α | scFv | CD8α | 4-1BB | CD27 | none | CD3ζ |
| 69 | CD8α | scFv | CD8α | 4-1BB | CD2 | none | CD3ζ |
| 70 | CD8α | scFv | CD8α | 4-1BB | OX40 | none | CD3ζ |
| 71 | CD8α | scFv | CD8α | 4-1BB | OX40L | none | CD3ζ |
| 72 | CD8α | scFv | CD8α | 4-1BB | DNAM-1 | none | CD3ζ |
| 73 | CD8α | scFv | CD8α | 4-1BB | NKp30 | none | CD3ζ |
| 74 | CD8α | scFv | CD8α | 4-1BB | NKp44 | none | CD3ζ |
| 75 | CD8α | scFv | CD8α | 4-1BB | NKp46 | none | CD3ζ |
| 76 | CD8α | scFv | CD8α | 4-1BB | ICOS | none | CD3ζ |
| 77 | CD8α | scFv | CD8α | 4-1BB | NKG2D | none | CD3ζ |
| 78 | CD8α | scFv | CD8α | 4-1BB | JAMAL | none | CD3ζ |
| 79 | CD8α | scFv | CD8α | 4-1BB | GITR | none | CD3ζ |
| 80 | CD8α | scFv | CD8α | 4-1BB | HVEM | none | CD3ζ |
| 81 | CD8α | scFv | CD8α | 4-1BB | TIM1 | none | CD3ζ |
| 82 | CD8α | scFv | CD8α | 4-1BB | LFA1 | none | CD3ζ |
| 83 | CD8α | scFv | CD8α | CD8α | CD28 | 4-1BB | CD3ζ |
| 84 | CD8α | scFv | CD8α | CD8α | CD28 | DAP10 | CD3ζ |
| 85 | CD8α | scFv | CD8α | CD8α | CD28 | DAP12 | CD3ζ |
| 86 | CD8α | scFv | CD8α | CD8α | CD28 | CD27 | CD3ζ |
| 87 | CD8α | scFv | CD8α | CD8α | CD28 | CD2 | CD3ζ |
| 88 | CD8α | scFv | CD8α | CD8α | CD28 | OX40 | CD3ζ |
| 89 | CD8α | scFv | CD8α | CD8α | CD28 | OX40L | CD3ζ |
| 90 | CD8α | scFv | CD8α | CD8α | CD28 | ICOS | CD3ζ |
| 91 | CD8α | scFv | CD8α | CD8α | CD28 | DNAM-1 | CD3ζ |
| 92 | CD8α | scFv | CD8α | CD8α | CD28 | NKp30 | CD3ζ |
| 93 | CD8α | scFv | CD8α | CD8α | CD28 | NKp44 | CD3ζ |
| 94 | CD8α | scFv | CD8α | CD8α | CD28 | NKp46 | CD3ζ |
| 95 | CD8α | scFv | CD8α | CD8α | CD28 | NKG2D | CD3ζ |
| 96 | CD8α | scFv | CD8α | CD8α | CD28 | JAMAL | CD3ζ |
| 97 | CD8α | scFv | CD8α | CD8α | CD28 | 2B4 | CD3ζ |
| 98 | CD8α | scFv | CD8α | CD8α | 4-1BB | CD28 | CD3ζ |
| 99 | CD8α | scFv | CD8α | CD8α | 4-1BB | DAP10 | CD3ζ |
| 100 | CD8α | scFv | CD8α | CD8α | 4-1BB | DAP12 | CD3ζ |
| 101 | CD8α | scFv | CD8α | CD8α | 4-1BB | CD27 | CD3ζ |
| 102 | CD8α | scFv | CD8α | CD8α | 4-1BB | CD2 | CD3ζ |
| 103 | CD8α | scFv | CD8α | CD8α | 4-1BB | OX40 | CD3ζ |
| 104 | CD8α | scFv | CD8α | CD8α | 4-1BB | OX40L | CD3ζ |
| 105 | CD8α | scFv | CD8α | CD8α | 4-1BB | ICOS | CD3ζ |
| 106 | CD8α | scFv | CD8α | CD8α | 4-1BB | DNAM-1 | CD3ζ |
| 107 | CD8α | scFv | CD8α | CD8α | 4-1BB | NKp30 | CD3ζ |
| 108 | CD8α | scFv | CD8α | CD8α | 4-1BB | NKp44 | CD3ζ |
| 109 | CD8α | scFv | CD8α | CD8α | 4-1BB | NKp46 | CD3ζ |
| 110 | CD8α | scFv | CD8α | CD8α | 4-1BB | NKG2D | CD3ζ |
| 111 | CD8α | scFv | CD8α | CD8α | 4-1BB | JAMAL | CD3ζ |
| 112 | CD8α | scFv | CD8α | CD8α | 4-1BB | 2B4 | CD3ζ |
| 113 | CD8α | scFv | CD8α | CD8α | 2B4 | 4-1BB | CD3ζ |
| 114 | CD8α | scFv | CD8α | CD8α | 2B4 | CD28 | CD3ζ |
| 115 | CD8α | scFv | CD8α | CD8α | 2B4 | DAP10 | CD3ζ |
| 116 | CD8α | scFv | CD8α | CD8α | 2B4 | DAP12 | CD3ζ |
| 117 | CD8α | scFv | CD8α | CD8α | 2B4 | CD27 | CD3ζ |
| 118 | CD8α | scFv | CD8α | CD8α | 2B4 | CD2 | CD3ζ |
| 119 | CD8α | scFv | CD8α | CD8α | 2B4 | OX40 | CD3ζ |
| 120 | CD8α | scFv | CD8α | CD8α | 2B4 | OX40L | CD3ζ |
| 121 | CD8α | scFv | CD8α | CD8α | 2B4 | ICOS | CD3ζ |
| 122 | CD8α | scFv | CD8α | CD8α | 2B4 | DNAM-1 | CD3ζ |
| 123 | CD8α | scFv | CD8α | CD8α | 2B4 | NKp30 | CD3ζ |
| 124 | CD8α | scFv | CD8α | CD8α | 2B4 | NKp44 | CD3ζ |
| 125 | CD8α | scFv | CD8α | CD8α | 2B4 | NKp46 | CD3ζ |
| 126 | CD8α | scFv | CD8α | CD8α | 2B4 | NKG2D | CD3ζ |
| 127 | CD8α | scFv | CD8α | CD8α | 2B4 | JAMAL | CD3ζ |
| 128 | CD8α | scFv | CD8α | CD28 | CD28 | 4-1BB | CD3ζ |
| 129 | CD8α | scFv | CD8α | CD28 | CD28 | DAP10 | CD3ζ |
| 130 | CD8α | scFv | CD8α | CD28 | CD28 | DAP12 | CD3ζ |
| 131 | CD8α | scFv | CD8α | CD28 | CD28 | CD27 | CD3ζ |
| 132 | CD8α | scFv | CD8α | CD28 | CD28 | CD2 | CD3ζ |
| 133 | CD8α | scFv | CD8α | CD28 | CD28 | OX40 | CD3ζ |
| 134 | CD8α | scFv | CD8α | CD28 | CD28 | OX40L | CD3ζ |
| 135 | CD8α | scFv | CD8α | CD28 | CD28 | ICOS | CD3ζ |
| 136 | CD8α | scFv | CD8α | CD28 | CD28 | DNAM-1 | CD3ζ |
| 137 | CD8α | scFv | CD8α | CD28 | CD28 | NKp30 | CD3ζ |
| 138 | CD8α | scFv | CD8α | CD28 | CD28 | NKp44 | CD3ζ |
| 139 | CD8α | scFv | CD8α | CD28 | CD28 | NKp46 | CD3ζ |
| 140 | CD8α | scFv | CD8α | CD28 | CD28 | NKG2D | CD3ζ |
| 141 | CD8α | scFv | CD8α | CD28 | CD28 | JAMAL | CD3ζ |
| 142 | CD8α | scFv | CD8α | CD28 | CD28 | 2B4 | CD3ζ |
| 143 | CD8α | scFv | CD8α | CD28 | 4-1BB | CD28 | CD3ζ |
| 144 | CD8α | scFv | CD8α | CD28 | 4-1BB | DAP10 | CD3ζ |
| 145 | CD8α | scFv | CD8α | CD28 | 4-1BB | DAP12 | CD3ζ |
| 146 | CD8α | scFv | CD8α | CD28 | 4-1BB | CD27 | CD3ζ |
| 147 | CD8α | scFv | CD8α | CD28 | 4-1BB | CD2 | CD3ζ |
| 148 | CD8α | scFv | CD8α | CD28 | 4-1BB | OX40 | CD3ζ |
| 149 | CD8α | scFv | CD8α | CD28 | 4-1BB | OX40L | CD3ζ |
| 150 | CD8α | scFv | CD8α | CD28 | 4-1BB | ICOS | CD3ζ |
| 151 | CD8α | scFv | CD8α | CD28 | 4-1BB | DNAM-1 | CD3ζ |
| 152 | CD8α | scFv | CD8α | CD28 | 4-1BB | NKp30 | CD3ζ |
| 153 | CD8α | scFv | CD8α | CD28 | 4-1BB | NKp44 | CD3ζ |
| 154 | CD8α | scFv | CD8α | CD28 | 4-1BB | NKp46 | CD3ζ |
| 155 | CD8α | scFv | CD8α | CD28 | 4-1BB | NKG2D | CD3ζ |
| 156 | CD8α | scFv | CD8α | CD28 | 4-1BB | JAMAL | CD3ζ |
| 157 | CD8α | scFv | CD8α | CD28 | 4-1BB | 2B4 | CD3ζ |
| 158 | CD8α | scFv | CD8α | CD28 | 2B4 | CD28 | CD3ζ |
| 159 | CD8α | scFv | CD8α | CD28 | 2B4 | DAP10 | CD3ζ |
| 160 | CD8α | scFv | CD8α | CD28 | 2B4 | DAP12 | CD3ζ |
| 161 | CD8α | scFv | CD8α | CD28 | 2B4 | CD27 | CD3ζ |
| 162 | CD8α | scFv | CD8α | CD28 | 2B4 | CD2 | CD3ζ |
| 163 | CD8α | scFv | CD8α | CD28 | 2B4 | OX40 | CD3ζ |
| 164 | CD8α | scFv | CD8α | CD28 | 2B4 | OX40L | CD3ζ |
| 165 | CD8α | scFv | CD8α | CD28 | 2B4 | ICOS | CD3ζ |
| 166 | CD8α | scFv | CD8α | CD28 | 2B4 | DNAM-1 | CD3ζ |
| 167 | CD8α | scFv | CD8α | CD28 | 2B4 | NKp30 | CD3ζ |
| 168 | CD8α | scFv | CD8α | CD28 | 2B4 | NKp44 | CD3ζ |
| 169 | CD8α | scFv | CD8α | CD28 | 2B4 | NKp46 | CD3ζ |
| 170 | CD8α | scFv | CD8α | CD28 | 2B4 | NKG2D | CD3ζ |
| 171 | CD8α | scFv | CD8α | CD28 | 2B4 | JAMAL | CD3ζ |
| 172 | CD8α | scFv | CD8α | CD28 | 2B4 | 2B4 | CD3ζ |
| 173 | CD8α | scFv | CD8α | 4-1BB | CD28 | 4-1BB | CD3ζ |
| 174 | CD8α | scFv | CD8α | 4-1BB | CD28 | DAP10 | CD3ζ |
| 175 | CD8α | scFv | CD8α | 4-1BB | CD28 | DAP12 | CD3ζ |
| 176 | CD8α | scFv | CD8α | 4-1BB | CD28 | CD27 | CD3ζ |
| 177 | CD8α | scFv | CD8α | 4-1BB | CD28 | CD2 | CD3ζ |
| 178 | CD8α | scFv | CD8α | 4-1BB | CD28 | OX40 | CD3ζ |
| 179 | CD8α | scFv | CD8α | 4-1BB | CD28 | OX40L | CD3ζ |
| 180 | CD8α | scFv | CD8α | 4-1BB | CD28 | ICOS | CD3ζ |
| 181 | CD8α | scFv | CD8α | 4-1BB | CD28 | DNAM-1 | CD3ζ |
| 182 | CD8α | scFv | CD8α | 4-1BB | CD28 | NKp30 | CD3ζ |
| 183 | CD8α | scFv | CD8α | 4-1BB | CD28 | NKp44 | CD3ζ |
| 184 | CD8α | scFv | CD8α | 4-1BB | CD28 | NKp46 | CD3ζ |
| 185 | CD8α | scFv | CD8α | 4-1BB | CD28 | NKG2D | CD3ζ |
| 186 | CD8α | scFv | CD8α | 4-1BB | CD28 | JAMAL | CD3ζ |
| 187 | CD8α | scFv | CD8α | 4-1BB | CD28 | 2B4 | CD3ζ |
| 188 | CD8α | scFv | CD8α | 4-1BB | 4-1BB | DAP10 | CD3ζ |
| 189 | CD8α | scFv | CD8α | 4-1BB | 4-1BB | DAP12 | CD3ζ |
| 190 | CD8α | scFv | CD8α | 4-1BB | 4-1BB | CD27 | CD3ζ |
| 191 | CD8α | scFv | CD8α | 4-1BB | 4-1BB | CD2 | CD3ζ |
| 192 | CD8α | scFv | CD8α | 4-1BB | 4-1BB | OX40 | CD3ζ |
| 193 | CD8α | scFv | CD8α | 4-1BB | 4-1BB | OX40L | CD3ζ |
| 194 | CD8α | scFv | CD8α | 4-1BB | 4-1BB | ICOS | CD3ζ |
| 195 | CD8α | scFv | CD8α | 4-1BB | 4-1BB | DNAM-1 | CD3ζ |
| 196 | CD8α | scFv | CD8α | 4-1BB | 4-1BB | NKp30 | CD3ζ |
| 197 | CD8α | scFv | CD8α | 4-1BB | 4-1BB | NKp44 | CD3ζ |
| 198 | CD8α | scFv | CD8α | 4-1BB | 4-1BB | NKp46 | CD3ζ |
| 199 | CD8α | scFv | CD8α | 4-1BB | 4-1BB | NKG2D | CD3ζ |
| 200 | CD8α | scFv | CD8α | 4-1BB | 4-1BB | JAMAL | CD3ζ |
| 201 | CD8α | scFv | CD8α | 4-1BB | 4-1BB | 2B4 | CD3ζ |
| 202 | CD8α | scFv | CD8α | 4-1BB | 2B4 | DAP10 | CD3ζ |
| 203 | CD8α | scFv | CD8α | 4-1BB | 2B4 | DAP12 | CD3ζ |
| 204 | CD8α | scFv | CD8α | 4-1BB | 2B4 | CD27 | CD3ζ |
| 205 | CD8α | scFv | CD8α | 4-1BB | 2B4 | CD2 | CD3ζ |
| 206 | CD8α | scFv | CD8α | 4-1BB | 2B4 | OX40 | CD3ζ |
| 207 | CD8α | scFv | CD8α | 4-1BB | 2B4 | OX40L | CD3ζ |
| 208 | CD8α | scFv | CD8α | 4-1BB | 2B4 | ICOS | CD3ζ |
| 209 | CD8α | scFv | CD8α | 4-1BB | 2B4 | DNAM-1 | CD3ζ |
| 210 | CD8α | scFv | CD8α | 4-1BB | 2B4 | NKp30 | CD3ζ |
| 211 | CD8α | scFv | CD8α | 4-1BB | 2B4 | NKp44 | CD3ζ |
| 212 | CD8α | scFv | CD8α | 4-1BB | 2B4 | NKp46 | CD3ζ |
| 213 | CD8α | scFv | CD8α | 4-1BB | 2B4 | NKG2D | CD3ζ |
| 214 | CD8α | scFv | CD8α | 4-1BB | 2B4 | JAMAL | CD3ζ |
| 215 | CD8α | scFv | CD8α | 4-1BB | 2B4 | 2B4 | CD3ζ |
| 216 | CD8α | scFv | CD8α | NKG2D | CD28 | CD28 | CD3ζ |
| 217 | CD8α | scFv | CD8α | NKG2D | CD28 | DAP10 | CD3ζ |
| 218 | CD8α | scFv | CD8α | NKG2D | CD28 | DAP12 | CD3ζ |
| 219 | CD8α | scFv | CD8α | NKG2D | CD28 | CD27 | CD3ζ |
| 220 | CD8α | scFv | CD8α | NKG2D | CD28 | CD2 | CD3ζ |
| 221 | CD8α | scFv | CD8α | NKG2D | CD28 | OX40 | CD3ζ |
| 222 | CD8α | scFv | CD8α | NKG2D | CD28 | OX40L | CD3ζ |
| 223 | CD8α | scFv | CD8α | NKG2D | CD28 | ICOS | CD3ζ |
| 224 | CD8α | scFv | CD8α | NKG2D | CD28 | DNAM-1 | CD3ζ |
| 225 | CD8α | scFv | CD8α | NKG2D | CD28 | NKp30 | CD3ζ |
| 226 | CD8α | scFv | CD8α | NKG2D | CD28 | NKp44 | CD3ζ |
| 227 | CD8α | scFv | CD8α | NKG2D | CD28 | NKp46 | CD3ζ |
| 228 | CD8α | scFv | CD8α | NKG2D | CD28 | NKG2D | CD3ζ |
| 229 | CD8α | scFv | CD8α | NKG2D | CD28 | JAMAL | CD3ζ |
| 230 | CD8α | scFv | CD8α | NKG2D | CD28 | 2B4 | CD3ζ |
| 231 | CD8α | scFv | CD8α | NKG2D | 4-1BB | DAP10 | CD3ζ |
| 232 | CD8α | scFv | CD8α | NKG2D | 4-1BB | DAP12 | CD3ζ |
| 233 | CD8α | scFv | CD8α | NKG2D | 4-1BB | CD27 | CD3ζ |
| 234 | CD8α | scFv | CD8α | NKG2D | 4-1BB | CD2 | CD3ζ |
| 235 | CD8α | scFv | CD8α | NKG2D | 4-1BB | OX40 | CD3ζ |
| 236 | CD8α | scFv | CD8α | NKG2D | 4-1BB | OX40L | CD3ζ |
| 237 | CD8α | scFv | CD8α | NKG2D | 4-1BB | ICOS | CD3ζ |
| 238 | CD8α | scFv | CD8α | NKG2D | 4-1BB | DNAM-1 | CD3ζ |
| 239 | CD8α | scFv | CD8α | NKG2D | 4-1BB | NKp30 | CD3ζ |
| 240 | CD8α | scFv | CD8α | NKG2D | 4-1BB | NKp44 | CD3ζ |
| 241 | CD8α | scFv | CD8α | NKG2D | 4-1BB | NKp46 | CD3ζ |
| 242 | CD8α | scFv | CD8α | NKG2D | 4-1BB | NKG2D | CD3ζ |
| 243 | CD8α | scFv | CD8α | NKG2D | 4-1BB | JAMAL | CD3ζ |
| 244 | CD8α | scFv | CD8α | NKG2D | 4-1BB | 2B4 | CD3ζ |
| 245 | CD8α | scFv | CD8α | NKG2D | 2B4 | CD28 | CD3ζ |
| 246 | CD8α | scFv | CD8α | NKG2D | 2B4 | DAP10 | CD3ζ |
| 247 | CD8α | scFv | CD8α | NKG2D | 2B4 | DAP12 | CD3ζ |
| 248 | CD8α | scFv | CD8α | NKG2D | 2B4 | CD27 | CD3ζ |
| 249 | CD8α | scFv | CD8α | NKG2D | 2B4 | CD2 | CD3ζ |
| 250 | CD8α | scFv | CD8α | NKG2D | 2B4 | OX40 | CD3ζ |
| 251 | CD8α | scFv | CD8α | NKG2D | 2B4 | OX40L | CD3ζ |
| 252 | CD8α | scFv | CD8α | NKG2D | 2B4 | ICOS | CD3ζ |
| 253 | CD8α | scFv | CD8α | NKG2D | 2B4 | DNAM-1 | CD3ζ |
| 254 | CD8α | scFv | CD8α | NKG2D | 2B4 | NKp30 | CD3ζ |
| 255 | CD8α | scFv | CD8α | NKG2D | 2B4 | NKp44 | CD3ζ |
| 256 | CD8α | scFv | CD8α | NKG2D | 2B4 | NKp46 | CD3ζ |
| 257 | CD8α | scFv | CD8α | NKG2D | 2B4 | NKG2D | CD3ζ |
| 258 | CD8α | scFv | CD8α | NKG2D | 2B4 | JAMAL | CD3ζ |

TABLE 13
Exemplary CAR Constructs for Expression in γδ T Cells

| | | Antigen | Hinge | Transmembr | Co-stim. | Co-stim. | Cytoplasmic |
|---|---|---|---|---|---|---|---|
| CAR | Signal | binding | domain | ane domain | domain | domain | Signaling |
| # | Sequence | domain | (e) | (b) | (d1) | (d2) | domain (c) |
| 1 | CD8α | scFv | CD8α | CD8α | 4-1BB | none | CD3ζ |
| 2 | CD8α | scFv | CD8α | CD28 | 4-1BB | none | CD3ζ |
| 3 | CD8α | scFv | CD8α | 4-1BB | 4-1BB | none | CD3ζ |
| 4 | CD8α | scFv | CD8α | CD4 | 4-1BB | none | CD3ζ |
| 5 | CD8α | scFv | CD8α | FcεRIγ | 4-1BB | none | CD3ζ |
| 6 | CD8α | scFv | CD8α | CD8β | 4-1BB | none | CD3ζ |
| 7 | CD8α | scFv | CD8α | C16α | 4-1BB | none | CD3ζ |
| 8 | CD8α | scFv | CD8α | OX40 | 4-1BB | none | CD3ζ |
| 9 | CD8α | scFv | CD8α | CD3ζ | 4-1BB | none | CD3ζ |
| 10 | CD8α | scFv | CD8α | CD3δ | 4-1BB | none | CD3ζ |
| 11 | CD8α | scFv | CD8α | CD3γ | 4-1BB | none | CD3ζ |
| 12 | CD8α | scFv | CD8α | CD3δ | 4-1BB | none | CD3ζ |
| 13 | CD8α | scFv | CD8α | TCR-α | 4-1BB | none | CD3ζ |
| 14 | CD8α | scFv | CD8α | CD32 | 4-1BB | none | CD3ζ |
| 15 | CD8α | scFv | CD8α | CD64 | 4-1BB | none | CD3ζ |
| 16 | CD8α | scFv | CD8α | VEGFR2 | 4-1BB | none | CD3ζ |
| 17 | CD8α | scFv | CD8α | FAS | 4-1BB | none | CD3ζ |
| 18 | CD8α | scFv | CD8α | FGFR2B | 4-1BB | none | CD3ζ |
| 19 | CD8α | scFv | CD8α | CD8α | CD2 | none | CD3ζ |
| 20 | CD8α | scFv | CD8α | CD8α | CD28 | none | CD3ζ |
| 21 | CD8α | scFv | CD8α | CD8α | CD27 | none | CD3ζ |
| 22 | CD8α | scFv | CD8α | CD8α | ICOS | none | CD3ζ |
| 23 | CD8α | scFv | CD8α | CD8α | JAMAL | none | CD3ζ |
| 24 | CD8α | scFv | CD8α | CD8α | OX40 | none | CD3ζ |
| 25 | CD8α | scFv | CD8α | CD8α | OX40L | none | CD3ζ |
| 26 | CD8α | scFv | CD8α | CD8α | NKG2D | none | CD3ζ |
| 27 | CD8α | scFv | CD8α | CD8α | CD46 | none | CD3ζ |
| 28 | CD8α | scFv | CD8α | CD8α | DNAM-1 | none | CD3ζ |
| 29 | CD8α | scFv | CD8α | CD8α | NKp30 | none | CD3ζ |
| 30 | CD8α | scFv | CD8α | CD8α | NKp44 | none | CD3ζ |
| 31 | CD8α | scFv | CD8α | CD8α | DAP10 | none | CD3ζ |
| 32 | CD8α | scFv | CD8α | CD28 | CD2 | none | CD3ζ |
| 33 | CD8α | scFv | CD8α | CD28 | 4-1BB | none | CD3ζ |
| 34 | CD8α | scFv | CD8α | CD28 | CD28 | none | CD3ζ |
| 35 | CD8α | scFv | CD8α | CD28 | CD27 | none | CD3ζ |
| 36 | CD8α | scFv | CD8α | CD28 | ICOS | none | CD3ζ |
| 37 | CD8α | scFv | CD8α | CD28 | JAMAL | none | CD3ζ |
| 38 | CD8α | scFv | CD8α | CD28 | OX40 | none | CD3ζ |
| 39 | CD8α | scFv | CD8α | CD28 | OX40L | none | CD3ζ |
| 40 | CD8α | scFv | CD8α | CD28 | NKG2D | none | CD3ζ |
| 41 | CD8α | scFv | CD8α | CD28 | CD46 | none | CD3ζ |
| 42 | CD8α | scFv | CD8α | CD28 | DNAM-1 | none | CD3ζ |
| 43 | CD8α | scFv | CD8α | CD28 | NKp30 | none | CD3ζ |
| 44 | CD8α | scFv | CD8α | CD28 | NKp44 | none | CD3ζ |
| 45 | CD8α | scFv | CD8α | CD28 | DAP10 | none | CD3ζ |
| 46 | CD8α | scFv | CD8α | 4-1BB | CD2 | none | CD3ζ |
| 47 | CD8α | scFv | CD8α | 4-1BB | 4-1BB | none | CD3ζ |
| 48 | CD8α | scFv | CD8α | 4-1BB | CD28 | none | CD3ζ |
| 49 | CD8α | scFv | CD8α | 4-1BB | CD27 | none | CD3ζ |
| 50 | CD8α | scFv | CD8α | 4-1BB | ICOS | none | CD3ζ |
| 51 | CD8α | scFv | CD8α | 4-1BB | JAMAL | none | CD3ζ |
| 52 | CD8α | scFv | CD8α | 4-1BB | OX40 | none | CD3ζ |
| 53 | CD8α | scFv | CD8α | 4-1BB | OX40L | none | CD3ζ |
| 54 | CD8α | scFv | CD8α | 4-1BB | NKG2D | none | CD3ζ |
| 55 | CD8α | scFv | CD8α | 4-1BB | CD46 | none | CD3ζ |
| 56 | CD8α | scFv | CD8α | 4-1BB | DNAM-1 | none | CD3ζ |
| 57 | CD8α | scFv | CD8α | 4-1BB | NKp30 | none | CD3ζ |
| 58 | CD8α | scFv | CD8α | 4-1BB | NKp44 | none | CD3ζ |
| 59 | CD8α | scFv | CD8α | 4-1BB | DAP10 | none | CD3ζ |
| 60 | CD8α | scFv | CD8α | CD8α | CD28 | 4-1BB | CD3ζ |
| 61 | CD8α | scFv | CD8α | CD8α | CD28 | CD2 | CD3ζ |
| 62 | CD8α | scFv | CD8α | CD8α | CD28 | CD27 | CD3ζ |
| 63 | CD8α | scFv | CD8α | CD8α | CD28 | ICOS | CD3ζ |
| 64 | CD8α | scFv | CD8α | CD8α | CD28 | JAMAL | CD3ζ |
| 65 | CD8α | scFv | CD8α | CD8α | CD28 | OX40 | CD3ζ |
| 66 | CD8α | scFv | CD8α | CD8α | CD28 | OX40L | CD3ζ |
| 67 | CD8α | scFv | CD8α | CD8α | CD28 | NKG2D | CD3ζ |
| 68 | CD8α | scFv | CD8α | CD8α | CD28 | CD46 | CD3ζ |
| 69 | CD8α | scFv | CD8α | CD8α | CD28 | DNAM-1 | CD3ζ |
| 70 | CD8α | scFv | CD8α | CD8α | CD28 | NKp30 | CD3ζ |
| 71 | CD8α | scFv | CD8α | CD8α | CD28 | NKp44 | CD3ζ |
| 72 | CD8α | scFv | CD8α | CD8α | CD28 | DAP10 | CD3ζ |
| 73 | CD8α | scFv | CD8α | CD8α | 4-1BB | CD2 | CD3ζ |
| 74 | CD8α | scFv | CD8α | CD8α | 4-1BB | CD27 | CD3ζ |
| 75 | CD8α | scFv | CD8α | CD8α | 4-1BB | ICOS | CD3ζ |
| 76 | CD8α | scFv | CD8α | CD8α | 4-1BB | JAMAL | CD3ζ |
| 77 | CD8α | scFv | CD8α | CD8α | 4-1BB | OX40 | CD3ζ |
| 78 | CD8α | scFv | CD8α | CD8α | 4-1BB | OX40L | CD3ζ |
| 79 | CD8α | scFv | CD8α | CD8α | 4-1BB | NKG2D | CD3ζ |
| 80 | CD8α | scFv | CD8α | CD8α | 4-1BB | CD46 | CD3ζ |
| 81 | CD8α | scFv | CD8α | CD8α | 4-1BB | DNAM-1 | CD3ζ |
| 82 | CD8α | scFv | CD8α | CD8α | 4-1BB | NKp30 | CD3ζ |
| 83 | CD8α | scFv | CD8α | CD8α | 4-1BB | NKp44 | CD3ζ |
| 84 | CD8α | scFv | CD8α | CD8α | 4-1BB | DAP10 | CD3ζ |
| 85 | CD8α | scFv | CD8α | CD28 | CD28 | 4-1BB | CD3ζ |
| 86 | CD8α | scFv | CD8α | CD28 | CD28 | CD2 | CD3ζ |
| 87 | CD8α | scFv | CD8α | CD28 | CD28 | CD27 | CD3ζ |
| 88 | CD8α | scFv | CD8α | CD28 | CD28 | ICOS | CD3ζ |
| 89 | CD8α | scFv | CD8α | CD28 | CD28 | JAMAL | CD3ζ |
| 90 | CD8α | scFv | CD8α | CD28 | CD28 | OX40 | CD3ζ |
| 91 | CD8α | scFv | CD8α | CD28 | CD28 | OX40L | CD3ζ |
| 92 | CD8α | scFv | CD8α | CD28 | CD28 | NKG2D | CD3ζ |
| 93 | CD8α | scFv | CD8α | CD28 | CD28 | CD46 | CD3ζ |
| 94 | CD8α | scFv | CD8α | CD28 | CD28 | DNAM-1 | CD3ζ |
| 95 | CD8α | scFv | CD8α | CD28 | CD28 | NKp30 | CD3ζ |
| 96 | CD8α | scFv | CD8α | CD28 | CD28 | NKp44 | CD3ζ |
| 97 | CD8α | scFv | CD8α | CD28 | CD28 | DAP10 | CD3ζ |
| 98 | CD8α | scFv | CD8α | CD28 | 4-1BB | CD2 | CD3ζ |
| 99 | CD8α | scFv | CD8α | CD28 | 4-1BB | CD27 | CD3ζ |
| 100 | CD8α | scFv | CD8α | CD28 | 4-1BB | ICOS | CD3ζ |
| 101 | CD8α | scFv | CD8α | CD28 | 4-1BB | JAMAL | CD3ζ |
| 102 | CD8α | scFv | CD8α | CD28 | 4-1BB | OX40 | CD3ζ |
| 103 | CD8α | scFv | CD8α | CD28 | 4-1BB | OX40L | CD3ζ |
| 104 | CD8α | scFv | CD8α | CD28 | 4-1BB | NKG2D | CD3ζ |
| 105 | CD8α | scFv | CD8α | CD28 | 4-1BB | CD46 | CD3ζ |
| 106 | CD8α | scFv | CD8α | CD28 | 4-1BB | DNAM-1 | CD3ζ |
| 107 | CD8α | scFv | CD8α | CD28 | 4-1BB | NKp30 | CD3ζ |
| 108 | CD8α | scFv | CD8α | CD28 | 4-1BB | NKp44 | CD3ζ |
| 109 | CD8α | scFv | CD8α | CD28 | 4-1BB | DAP10 | CD3ζ |
| 110 | CD8α | scFv | CD8α | 4-1BB | CD28 | CD2 | CD3ζ |
| 111 | CD8α | scFv | CD8α | 4-1BB | CD28 | CD27 | CD3ζ |
| 112 | CD8α | scFv | CD8α | 4-1BB | CD28 | ICOS | CD3ζ |
| 113 | CD8α | scFv | CD8α | 4-1BB | CD28 | JAMAL | CD3ζ |
| 114 | CD8α | scFv | CD8α | 4-1BB | CD28 | OX40 | CD3ζ |
| 115 | CD8α | scFv | CD8α | 4-1BB | CD28 | OX40L | CD3ζ |
| 116 | CD8α | scFv | CD8α | 4-1BB | CD28 | NKG2D | CD3ζ |
| 117 | CD8α | scFv | CD8α | 4-1BB | CD28 | CD46 | CD3ζ |
| 118 | CD8α | scFv | CD8α | 4-1BB | CD28 | DNAM-1 | CD3ζ |
| 119 | CD8α | scFv | CD8α | 4-1BB | CD28 | NKp30 | CD3ζ |
| 120 | CD8α | scFv | CD8α | 4-1BB | CD28 | NKp44 | CD3ζ |
| 121 | CD8α | scFv | CD8α | 4-1BB | CD28 | DAP10 | CD3ζ |
| 122 | CD8α | scFv | CD8α | 4-1BB | 4-1BB | CD2 | CD3ζ |
| 123 | CD8α | scFv | CD8α | 4-1BB | 4-1BB | CD27 | CD3ζ |
| 124 | CD8α | scFv | CD8α | 4-1BB | 4-1BB | ICOS | CD3ζ |
| 125 | CD8α | scFv | CD8α | 4-1BB | 4-1BB | JAMAL | CD3ζ |
| 126 | CD8α | scFv | CD8α | 4-1BB | 4-1BB | OX40 | CD3ζ |
| 127 | CD8α | scFv | CD8α | 4-1BB | 4-1BB | OX40L | CD3ζ |
| 128 | CD8α | scFv | CD8α | 4-1BB | 4-1BB | NKG2D | CD3ζ |
| 129 | CD8α | scFv | CD8α | 4-1BB | 4-1BB | CD46 | CD3ζ |
| 130 | CD8α | scFv | CD8α | 4-1BB | 4-1BB | DNAM-1 | CD3ζ |
| 131 | CD8α | scFv | CD8α | 4-1BB | 4-1BB | NKp30 | CD3ζ |
| 132 | CD8α | scFv | CD8α | 4-1BB | 4-1BB | NKp44 | CD3ζ |
| 133 | CD8α | scFv | CD8α | 4-1BB | 4-1BB | DAP10 | CD3ζ |

TABLE 14
Exemplary GPC3-Targeting CAR Constructs

| | Extracellular | | | Co- | |
|---|---|---|---|---|---|
| | domain | | Trans- | stimu- | Cytoplasmic |
| Signal | (antigen | Hinge | membrane | latory | Signaling |
| Sequence | binding) | domain | domain | domain | domain |
| CD8α | scFv (e.g., anti- | CD8α | CD8 | 4-1BB | CD3ζ |
| | GPC3 scFv) | | | | |
| CD8α | scFv (e.g., anti- | CD28 | CD28 | CD28 | CD3ζ |
| | GPC3 scFv) | | | | |

Amino acid sequences of an exemplary anti-GPC3 scFv for constructing anti-GPC CAR constructs, as well as exemplary anti-GPC3 CAR constructs comprising such are provided below:

Anti-GPC3scFv
(SEQ ID NO: 77)
DVVMTQSPLSLPVTPGEPASISCRSSQSLVHSNRNTYLHWYLQKPGQSPQ
LLIYKVSNRFSGVPDRFSGSGSGTDFTLKISRVEAEDVGVYYCSQNTHVP
PTFGQGTKLEIKRGGGGSGGGGSGGGGSQVQLVQSGAEVKKPGASVKVSC
KASGYTFTDYEMHWVRQAPGQGLEWMGALDPKTGDTAYSQKFKGRVTLTA
DKSTSTAYMELSSLTSEDTAVYYCTRFYSYTYWGQGTLVTVSS
Anti-GPC3-CAR1
(SEQ ID NO: 78)
MALPVTALLLPLALLLHAARPDVVMTQSPLSLPVTPGEPASISCRSSQSL
VHSNRNTYLHWYLQKPGQSPQLLIYKVSNRFSGVPDRESGSGSGTDFTLK
ISRVEAEDVGVYYCSQNTHVPPTFGQGTKLEIKRGGGGSGGGGSGGGGSQ
VQLVQSGAEVKKPGASVKVSCKASGYTFTDYEMHWVRQAPGQGLEWMGAL
DPKTGDTAYSQKFKGRVTLTADKSTSTAYMELSSLTSEDTAVYYCTRFYS
YTYWGQGTLVTVSSTTTPAPRPPTPAPTIASQPLSLRPEACRPAAGGAVH
TRGLDFACDIYIWAPLAGTCGVLLLSLVITLYCKRGRKKLLYIFKQPFMR
PVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR
Anti-GPC3-CAR2
(SEQ ID NO: 79)
MALPVTALLLPLALLLHAARPDVVMTQSPLSLPVTPGEPASISCRSSQSL
VHSNRNTYLHWYLQKPGQSPQLLIYKVSNRFSGVPDRESGSGSGTDFTLK
ISRVEAEDVGVYYCSQNTHVPPTFGQGTKLEIKRGGGGSGGGGSGGGGSQ
VQLVQSGAEVKKPGASVKVSCKASGYTFTDYEMHWVRQAPGQGLEWMGAL
DPKTGDTAYSQKFKGRVTLTADKSTSTAYMELSSLTSEDTAVYYCTRFYS
YTYWGQGTLVTVSSIEVMYPPPYLDNEKSNGTIIHVKGKHLCPSPLFPGP
SKPFWVLVVVGGVLACYSLLVTVAFIIFWVRSKRSRLLHSDYMNMTPRRP
GPTRKHYQPYAPPRDFAAYRSRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR

### I. TCR Polypeptides

The instant disclosure provides a modified immune cell (e.g., αβ T cells, γδ T cells or NKT cell) comprising an exogenous T cell receptor (TCR) on a target cell (e.g., recognizes peptide antigens presented on major histocompatibility (MHC) molecules associated with a disease such as cancer, see discussions herein). As used herein, a TCR complex is expressed on T or NKT cells and associates with a CD3 complex. Engagement of the TCR complex leads to T cell activation in the form of proliferation, cytokine secretion, and cytolytic activity. Provided herein are T cell receptor (TCR)-engineered immune cells (e.g., T and NKT cells) that can be used to improve cellular immunotherapy. For example, the disclosure provides modified immune cells containing a vector that encodes the chimeric polypeptide comprising (i) an extracellular single-chain variant fragment that specifically binds an epitope (e.g., tumor (neo)epitope or a tumor associated antigen). (ii) an intracellular activation domain, and (iii) a transmembrane linker coupling the extracellular single-chain variant fragment to the intracellular activation domain. Most typically, the first, second, and third segments are arranged such that the extracellular single-chain variant fragment, the intracellular activation domain, and the linker form a single chimeric polypeptide.

### (i) Peptide-MHC Complex

Major histocompatibility complex (MHC) refers to glycoproteins that deliver peptide antigens to a cell surface. Human MHC is referred to as human leukocyte antigen (HLA). MHC class I molecules are heterodimers having a membrane spanning α chain (with three α domains) and a non-covalently associated β2 microglobulin. MHC I consist of HLAs A, B and C and B2 microglobulin. MHC class II molecules are composed of two transmembrane glycoproteins, α and β, both of which span the membrane. Each chain has two domains. MHC II are heterodimers of several HLAs (HLA-DP, DQ, DR). MHC class I molecules deliver peptides originating in the cytosol to the cell surface, where a peptide-MHC complex is recognized by CD8⁺ T cells. MHC class II molecules deliver peptides originating in the vesicular system to the cell surface, where they are recognized by CD4⁺ T cells. TCRs can recognize a range of peptide antigens including tumor associated antigens (TAAs), cancer germline antigens (CGAs), and tumor specific antigens (TSAs) including viral antigens and neoantigens (enlisted in Table 1 of Shafer et al., Front Immunol, 13: 835762 (2022)).

### (ii) CD3 Complex

CD3, as used herein, refers to a multi-protein complex of six chains that is associated with antigen signaling in T cells. In mammals, the complex comprises a CD3 γ chain, a CD3 δ chain, two CD3 ε chains, and a homodimer of CD3 ζ chains. The CD3 γ, CD3 β, and CD3 ε chains are highly related cell surface proteins of the immunoglobulin superfamily containing a single immunoglobulin domain. The transmembrane regions of the CD3 γ, CD3 β, and CD3 ε chains are negatively charged, which is a characteristic that allows these chains to associate with the positively charged TCR chains. The intracellular tails of the CD3 γ, CD3 β, and CD3 ε chains each contain a single conserved motif known as an immunoreceptor tyrosine-based activation motif or ITAM, whereas each CD3ζ chain has three. Without wishing to be bound by theory, it is believed that the ITAMs are important for the signaling capacity of a TCR complex. CD3 as used in the present disclosure is preferably, the endogenous complex of the T or NKT cells used for the cellular therapy. Next, T or NKT cells may be from various animal species, including human, mouse, rat, or other mammals.

### (ii) TCR Complex

The TCR complex, as used herein, refers to a complex formed by the association of CD3 with TCR. TCR consists of two heterodimeric TCR chains, TCR α, TCR β (in αβ T cells) or TCR γ, TCR δ (in γδ T cells) and six CD3 chains which for a multiprotein complex that recognizes peptide antigens presented on MHC. The TCR complex forms the ligand binding site, and CD3 complex proteins mediate signaling and subsequent T cell activation. The TCRα is encoded by TRA gene, β chain encoded by TRB, γ chain encoded by TRG and δ chain encoded by TRD.

A component of a physiologic TCR complex refers to a TCR chain (i.e., TCR α, TCR β, TCR γ or TCR δ), a CD3 chain (i.e., CD3 γ, CD3 β, CD3 ε or CD3 ζ), or a complex formed by two or more TCR chains or CD3 chains (e.g., a complex of TCR α and TCR β, a complex of TCR γ and TCR δ, a complex of CD3 ε and CD3 δ, a complex of CD3 γ and CD3 ε, or a sub-TCR complex of TCR α, TCR β, CD3 γ, CD3 δ, and two CD3 ε chains). In some instances, the complex may be formed by two or more TCR chains or CD3 chains (e.g., a complex of TCR α and TCR β, a complex of TCR γ and TCR δ, a complex of CD3ε and CD3 δ, a complex of CD3 γ and CD3 ε, or a sub-TCR complex of TCR α, TCR β, CD3 γ, CD3 δ, and two CD3 ε chains).

The TCR is comprised of a heterodimeric TCR that are joined by disulfide bonds (α/β or γ/δ TCR) and forms a non-covalent multiprotein complex with the CD3 chains. The TCR chains are type I proteins consisting of an extracellular region, transmembrane region and a short cytoplasmic tail. The extracellular domain contains a hypervariable V region responsible for antigen recognition and a constant C region that is membrane proximal. The transmembrane and cytoplasmic domains form non-covalent interactions with the CD3 chains to stabilize the TCR complex and mediate downstream signaling (Kuhns et al., Immunol Rev, 250 (2012); Wucherpfennig et al., Cold Spring Harb Perspect Biol, 2: a005140 (2010)). A review of TCRs and their design is provided in Blankenstein et al., Curr Opin Immunol, 33: 112-9 (2015) (herein incorporated by reference for the subject matter and purpose referenced herein). Methods for producing engineered TCRs are described in, e.g., Bowerman et al., Mol Immunol, 46: 3000-8 (2009), the techniques of which are herein incorporated by reference. Furthermore, TCRs that bind a particular antigen may be isolated using a Vα or Vβ domain from a TCR that binds the antigen to screen a library of complementary Vα or Vβ domains, respectively. In certain embodiments, a TCR is found on the surface of T cells and associates with the CD3 complex. The source of a TCR as used in the present disclosure may be from various animal species, such as a human, mouse, rat, rabbit or other mammal.

The TCR complex further comprises an (a) extracellular domain; (b) transmembrane domain; and (c) Cytoplasmic domain. The extracellular domain may be derived either from a natural or from a recombinant source. Where the source is natural, the domain may be derived from any protein, but a membrane-bound or transmembrane protein. In one aspect the extracellular domain is capable of associating with the transmembrane domain. Non-limiting examples of an extracellular domain of particular use in this present disclosure may include that of α, β, γ, or δ chain of the TCR, or CD3 ε, CD3 γ, or CD3 δ, or in alternative embodiments, CD28, CD45, CD4, CD5, CD8, CD9, CD16, CD22, CD33, CD37, CD64, CD80, CD86, CD134, CD137, CD154.

In some embodiments, the antigen binding domain can comprise one member of an interacting pair, e.g., the antigen binding domain may be one member, or a fragment thereof, of an interacting pair comprising a receptor and a ligand. Either the receptor or ligand, or fragments thereof, may be referred to as the antigen binding domain. The other member which is not referred to as the antigen binding domain can comprise the epitope to which the antigen binding domain specifically binds.

The second antigen binding domain can be linked to any member of the TCR complex, and the TCR may be an α/β or γ/δ TCR. The second antigen binding domain can be linked to at least one of a TCR chain, a cluster of differentiation 3 (CD3) chain, or CD3 z chain. The second antigen binding domain can be linked to transmembrane receptor of a TCR, e.g., TCR δ, TCR γ, TCR α, or TCR β. The second antigen binding domain can be linked to a CD3 chain, e.g., CD3 ε, CD3 δ, or CD3 γ. The second antigen binding domain can be linked to CD3 z chain.

In some embodiments, a modified TCR complex comprises a first antigen binding domain fused to CD3 ε chain, and a second antigen binding domain fused to a CD3 δ chain. In some embodiments, a modified TCR complex comprises a first antigen binding domain fused to a CD3 δ chain, and a second antigen binding domain fused to a CD3 γ chain. In some embodiments, a modified TCR complex comprises a first antigen binding domain fused to a CD3 a chain or a CD3 β chain, and a second antigen binding domain fused to a CD3 ε chain. In some embodiments, a modified TCR complex comprises a first antigen binding domain fused to a CD3 β chain or a CD3 δ chain, and a second antigen binding domain fused to a CD3 ε chain. In some embodiments, a modified TCR complex comprises a first antigen binding domain fused to an α chain or a TCR γ chain, and a second antigen binding domain fused to a CD3 γ chain. In some embodiments, a modified TCR complex comprises a first antigen binding domain fused to a TCR β chain or a TCR δ chain, and a second antigen binding domain fused to a CD3 γ chain. In some embodiments, a modified TCR complex comprises a first antigen binding domain fused to a TCR α chain or a TCR γ chain, and a second antigen binding domain fused to a CD3 δ chain. In some embodiments, a modified TCR complex comprises a first antigen binding domain fused to a TCR β chain or a TCR δ chain, and a second antigen binding domain fused to a δ chain.

Next, the transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the TCR complex has bound to a target (i.e., peptide-MHC). Non-limiting examples of a transmembrane domain of particular use in this present disclosure may include α, β, γ or δ chain of the TCR, CD28, CD3 ε, CD3 γ, CD3 δ CD3 z, CD45, CD4, CD5, CD8, CD9, CD 16, CD22, CD33, CD37, CD64, CD80, CD86, CD134, CD137, CD154. The transmembrane domain can include one or more additional amino acids adjacent to the transmembrane region, e.g., one or more amino acid associated with the extracellular region of the protein from which the transmembrane was derived. In one aspect, the transmembrane domain is one that is associated with one of the other domains of the TCR used. In some instances, the transmembrane domain can be selected or modified by amino acid substitution to avoid binding of such domains to the transmembrane domains of the same or different surface membrane proteins, e.g., to minimize interactions with other members of the receptor complex.

In general, an extracellular domain and a transmembrane domain may be encoded by a single genomic sequence. In alternative embodiments, the sequence can be designed to comprise a transmembrane domain that is heterologous to the extracellular domain.

Optionally, a short oligo- or polypeptide linker, between 2 and 10 amino acids in length may form the linkage between the transmembrane domain and the cytoplasmic region of the TCR polypeptide. A glycine-serine doublet provides a particularly suitable linker (e.g., SEQ ID NO: 15-17).

The cytoplasmic domain of the TCR can include an intracellular domain. In some embodiments, the intracellular domain is from CD3 γ, CD3 δ, CD3 ε, TCR α, TCR β, TCR γ or TCR S. In some embodiments, the intracellular domain comprises a signaling domain, if the TCR complex contains CD3 γ, δ, ε polypeptides; TCR α, TCR β, TCR γ, and TCR δ subunits generally have short (e.g., 1-19 amino acids in length) intracellular domains and are generally lacking in a signaling domain. An intracellular signaling domain is generally responsible for activation of at least one of the normal effector functions of the immune cell in which the TCRs has been introduced. While the intracellular domains of TCR α, TCR β, TCR γ, and TCR δ do not have signaling domains, they are able to recruit proteins having a primary intracellular signaling domain described herein, e.g., CD3 z, which functions as an intracellular signaling domain.

In some instances, the TCR subunit comprises (i) at least a portion of a TCR extracellular domain, (ii) a TCR transmembrane domain, and (iii) a TCR intracellular domain, wherein at least two of (i), (ii), and (iii) are from the same TCR subunit. In some instances, the TCR extracellular domain comprises an extracellular domain or portion thereof of a TCR α chain, a TCR β chain, a TCR γ chain, a TCR δ chain, a CD3 ε TCR subunit, a CD3 γ TCR subunit, a CD3 δ TCR subunit and functional fragments thereof.

In some instances, the TCR subunit comprises a transmembrane domain comprising a transmembrane domain of a TCR α chain, a TCR β chain, a TCR γ chain, a TCR δ chain, a CD3 z TCR subunit, a CD3 ε TCR subunit, a CD3 γ TCR subunit, a CD3 δ TCR subunit, CD45, CD4, CD5, CD8, CD9, CD16, CD22, CD33, CD28, CD37, CD64, CD80, CD86, CD134, CD137, CD154 and functional fragments thereof.

In some instances, the TCR subunit comprises a TCR intracellular domain of CD3 ε, CD3 γ, CD3 δ, TCR α, TCR β, TCR γ, or TCR δ. In some embodiments, the intracellular domain comprises a stimulatory domain of a protein comprising an intracellular signaling domain of CD3 ε, CD3 γ, CD3 δ and functional fragments thereof.

In various embodiments of the aspects herein, a modified TCR complex comprises a TCR previously identified. In some cases, the TCR can be identified using whole-exomic sequencing. For example, a TCR can target a neoantigen or neoepitope that is identified by whole-exomic sequencing of a target cell. Alternatively, the TCR can be identified from autologous, allogenic, or xenogeneic repertoires.

A modified T cell receptor (TCR) complex can comprise a second antigen binding domain which exhibits binding to a second epitope. The second antigen binding domain can comprise any protein or molecule that can bind to an epitope. In some embodiments, the second antigen binding domain comprises a heterologous sequence exhibiting binding to the second epitope. Non-limiting examples of the second antigen binding domain of the TCR complex include, but are not limited to, a monoclonal antibody, a polyclonal antibody, a recombinant antibody, a human antibody, a humanized antibody, or a functional derivative, variant or fragment thereof, including, but not limited to, a Fab, a Fab′, a F (ab′) 2, an Fv, a single-chain Fv (scFv), minibody, a diabody, and a single-domain antibody such as a heavy chain variable domain (VH), a light chain variable domain (VL) and a variable domain (VH H) of camelid derived Nanobody. In some embodiments, the second antigen binding domain comprises a single-domain antibody (sdAb).

TCRs naturally have relatively low affinities due to thymic selection ranging around 1-100 μM. Amino acid substitutions in the variable regions can increase affinity into the low μM and nM ranges. Yeast and bacteriophage display methods have been used to generate very high affinity TCRs in the pM ranges. Increasing TCR affinity can also lead to a loss of specificity, however more nuanced structure-guided design approaches can be used to increase affinity without decreasing specificity. TCRs can also be engineered into single chain proteins containing variable regions from the TCRα and TCRβ chains fused by a peptide linker for antigen recognition domain and the CD3z domain to impart signaling. Additional costimulatory domains can also be included to improve signaling (Willemsen et al., Gene Ther, 7: 1369-77 (2000); Zhang et al., Cancer Gene Ther, 11: 487-96 (2004); Plaksin et al., J Immunol, 158: 2218-27 (1997); Chung et al., Proc Natl Acad Sci USA, 91: 12654-8 (1994)).

III. Hematopoietic Cells Expressing Factors that Redirect Glucose Metabolites and Optionally Chimeric Receptor Polypeptides

Provided herein are genetically engineered host cells (e.g., hematopoietic cells such as HSCs and immune cells, e.g., T cells or NK cells) expressing one or more of the factors that redirect glucose metabolites (e.g., redirect glucose metabolites out of the glycolysis pathway) as described herein. In some embodiments, the factors (e.g., polypeptides) are encoded by a transgene introduced into the host cells (e.g., exogenous to the host cells). The genetically engineered host cells may further express a chimeric receptor polypeptide (e.g., ACTR-expressing cells, e.g., ACTR T cells, CAR-expressing cells, e.g., CAR-T cells or TCR-expressing cells, e.g., TCR-T cells) as also described herein. In some embodiments, the host cells are hematopoietic cells or a progeny thereof. In some embodiments, the hematopoietic cells can be hematopoietic stem cells. In some embodiments, the genetically engineered immune cells can be natural killer (NK) cells, Natural Killer T (NKT) cells, monocytes/macrophages, neutrophils, eosinophils, αβ T or γδ T cells. In other embodiments, the host cells are immune cells, such as αβ T cells, γδ T cells (e.g, naive T cell, effector memory T cell, central memory T cell, a double negative T cell, an effector T cell, ThI cell, ThII cell, Th17 cell, a Th22 cell) or NK cells. In some embodiments, the immune cells are αβ T cells. In some embodiments, the immune cells are γδ T cells. In some embodiments, the γδ T cell is Vγ9δ2 T cell. In some embodiments, the γδ T cell is Vδ1 T cell. In some embodiments, the γδ T cell comprises Vγ9δ2 TCR, Vγ10/Vδ2 TCR, and/or Vγ2/Vδ2 TCR. In some embodiments, the immune cells are NK cells. In some embodiments, the immune cells are NKT cells.

In a preferred embodiment, the immune cell is an αβ T cell, and wherein the chimeric receptor polypeptide is a CAR polypeptide that comprises components as shown in Table 11 In another preferred embodiment, the immune cell is a NK cell, and wherein the chimeric receptor polypeptide is a CAR polypeptide that comprises components as shown in Table 12. In yet another preferred embodiment the immune cell is a γδ T cell, and wherein the chimeric receptor polypeptide is a CAR polypeptide that comprises components as shown in Table 13.

In some other embodiments, the genetically engineered immune cells described herein can be derived from a cell line, e.g., selected from NK-92, NK-92MI, YTS, and KHYG-1, preferably NK-92 cells. In other embodiments, the genetically engineered immune cells described herein can be derived from peripheral blood mononuclear cells (PBMC), hematopoietic stem cells (HSCs), cord blood stem cells (CBSCs) or induced pluripotent stem cells (iPSCs).

In some embodiments, the genetically engineered hematopoietic cells such as HSCs or immune cells (e.g., T cells or NK cells) may co-express any of the CAR constructs such as those disclosed herein with any of the factors that redirect glucose metabolites, such as a polypeptide that diverts or redirects glucose metabolites (e.g., PKM2, GFPT1, or TIGAR). In some embodiments, the CAR construct may comprise a co-stimulatory domain from 4-1BB or CD28 and the polypeptide that diverts or redirects glucose metabolites is PKM2, GFPT1, or TIGAR. The CAR construct may further comprise a hinge and transmembrane domain from CD8 (e.g., CD8α) or CD28.

In some examples, the genetically engineered hematopoietic cells such as HSCs or immune cells (e.g., T cells or NK cells) may be engineered to co-express any of the CAR constructs (e.g., the anti-GPC3 CAR disclosed herein) and TIGAR. In specific embodiments, the genetically engineered cells comprise T cells co-expressing the CAR and TIGAR. In other embodiments, the genetically engineered cells comprise NK cells co-expressing the CAR and TIGAR.

In other embodiments, the genetically engineered hematopoietic cells such as HSCs or immune cells (e.g., T cells or NK cells) may co-express any of the ACTR constructs such as those disclosed herein with any of the factors that redirect glucose metabolites, such as a polypeptide that diverts or redirects glucose metabolites (e.g., PKM2, GFPT1, or TIGAR). In some embodiments, the ACTR construct may comprise a co-stimulatory domain from 4-1BB or CD28 and the polypeptide that diverts or redirects glucose metabolites is PKM2, GFPT1, or TIGAR. The ACTR constructs may further comprise a hinge and transmembrane domain from CD8 or CD28.

In some examples, the genetically engineered hematopoietic cells such as HSCs or immune cells (e.g., T cells or NK cells) may be engineered to co-express any of the ACTR constructs (e.g., the CD16A-V158 ACTR disclosed herein) and TIGAR. In specific embodiments, the genetically engineered cells comprise T cells co-expressing the ACTR and TIGAR. In other embodiments, the genetically engineered cells comprise NK cells co-expressing the ACTR and TIGAR.

Alternatively, the genetically engineered host cells disclosed herein may not express any chimeric receptor polypeptides.

In some embodiments, the genetically engineered immune cells, which may overly express one or more factors that redirect glucose metabolites as disclosed herein, may be derived from tumor-infiltrating lymphocytes (TILs). Overexpression of the factor that redirects glucose metabolites may enhance the anti-tumor activity or the TILs in tumor microenvironment. In a specific embodiment TILs are selected that are reactive/target to a specific peptide presented an MHC complex.

The TILs and/or T cells expressing genetically modified TCRs may target a peptide-MHC complex, in which the peptide may be derived from a pathogen, a tumor antigen, or an autoantigen. Some examples are provided in Table 15 below.

Any of the CAR constructs disclosed herein or an antibody to be co-used with ACTR T cells may also target any of the peptide in such peptide/MHC complex.

In other embodiments, the genetically engineered hematopoietic cells such as HSCs or immune cells (e.g., T cells or NKT cells) may co-express TCR polypeptides with any of the factors that redirect glucose metabolites, such as a polypeptide that diverts or redirects glucose metabolites (e.g., PKM2, GFPT1, or TIGAR).

TABLE 15
Exemplary Peptide-MHC Targets

| Targets | Indications |
|---|---|
| NY-ESO-1 | Sarcoma, MM |
| MAGE-A10 | NSCLC, Bladder, HNSCC |
| MAGE-A4 | Sarcomas, others |
| PMEL | Melanoma |
| WT-1 | Ovarian |
| AFP | HCC |
| HPV-16 E6 | Cervical |
| HPV-16 E7 | Cervical |

In some embodiments, the host cells are immune cells, such as T cells or NK cells. In some embodiments, the immune cells are T cells. For example, the T cells can be CD4+ helper cells or CD8⁺ cytotoxic cells, or a combination thereof. Alternatively, or in addition, the T cells can be suppressive T cells such as Ts cells. In some embodiments, the immune cells are NK cells. In other embodiments, the immune cells can be derived cell lines, for example, NK-92 cells. The population of immune cells can be obtained from any source, such as peripheral blood mononuclear cells (PBMCs), bone marrow, or tissues such as spleen, lymph node, thymus, or tumor tissue. In one embodiment, lymphocytes are obtained from tumor tissue, i.e., tumor infiltrating lymphocytes (TILs). Alternatively, the immune cell population may be derived from stem cells, for example, hematopoietic stem cells (HSCs), cord blood stem cells and induced pluripotent stem cells (iPSCs). A source suitable for obtaining the type of host cells desired would be evident to one of skill in the art. In some examples, the immune cells can be a mixture of different types of T cells and/or NK cells as known in the art. For example, the immune cells can be a population of immune cells isolated from a suitable donor (e.g., a human patient). In a preferred embodiment, the population of immune cells is derived from PBMCs, which may be obtained from a patient (e.g., a human patient) who needs the treatment described herein. The type of host cells desired (e.g., T cells or NK cells) may be expanded within the population of cells obtained by co-incubating the cells with stimulatory molecules. As a non-limiting example, anti-CD3 and anti-CD28 antibodies may be used for expansion of T cells.

In further embodiments of the invention the genetically engineered immune cell comprises a nucleic acid or nucleic acid set, which collectively comprises a first nucleotide sequence encoding the factor that diverts or redirects glucose metabolites; and a second nucleotide sequence encoding the chimeric receptor polypeptide. In some instances, the factor that redirects glucose metabolites to be introduced into the host cells is identical to an endogenous protein of the host cell. Introducing additional copies of the coding sequences of the factor that redirects glucose metabolites into the host cell would enhance the expression level of the polypeptide (i.e., overly expressed) as relative to the native counterpart. In some instances, the factor that redirects glucose metabolites to be introduced into the host cells is heterologous to the host cell, i.e., does not exist or is not expressed in the host cell. Such a heterologous factor that redirects glucose metabolites may be a naturally-occurring protein not expressed in the host cell in nature (e.g., from a different species, or from a different cell type of the same species). Alternatively, the heterologous factor that redirects glucose metabolites may be a variant of a native protein, such as those described herein. In some examples, the exogenous (i.e., not native to the host cells) copy of the coding nucleic acid may exist extrachromosomally. In other examples, the exogenous copy of the coding sequence may be integrated into the chromosome of the host cell, and may be located at a site that is different from the native loci of the endogenous gene.

Such genetically engineered host cells have the capacity to have an enhanced rate of glycolysis and may, for example, have an enhanced capacity of taking glucose from the environment. Thus, these genetically engineered host cells may exhibit better growth and/or bioactivities under low glucose, low amino acid, low pH, and/or hypoxic conditions, for example in a tumor microenvironment.

The genetically engineered cells, when expressing a chimeric receptor polypeptide as disclosed herein, can recognize and inhibit target cells, either directly (e.g., by CAR-expressing immune cells or TCR-expressing T cells) or via an Fc-containing therapeutic agents such as an anti-tumor antibodies (e.g., by ACTR-expressing immune cells). Given their expected high proliferation rate, bioactivity, and/or survival rate in low glucose, low amino acid, low pH, and/or hypoxic environments (e.g., in a tumor microenvironment), the genetically engineered cells such as T cell, NKT and NK cells would be expected to have higher therapeutic efficacy relative to chimeric receptor polypeptide T, NKT or NK cells that do not express or express a lower level or less active form of the factor that redirects glucose metabolites.

To construct the immune cells that express any of the factors that redirect glucose metabolites and optionally the chimeric receptor polypeptide described herein, expression vectors for stable or transient expression of the factors that redirect glucose metabolites and/or the chimeric receptor polypeptide may be created via conventional methods as described herein and introduced into immune host cells. For example, nucleic acids encoding the factors that redirect glucose metabolites and/or the chimeric receptor polypeptides may be cloned into one or two suitable expression vectors, such as a viral vector or a non-viral vector in operable linkage to a suitable promoter. In some instances, each of the coding sequences for the chimeric receptor polypeptide and the factor that redirects glucose metabolites are on two separate nucleic acid molecules and can be cloned into two separate vectors, which may be introduced into suitable host cells simultaneously or sequentially. In other embodiments, the coding sequences for the chimeric receptor polypeptide and the factor that redirects glucose metabolites are on one nucleic acid molecule and can be cloned into one vector. Accordingly, it is one embodiment that the immune cell comprises the nucleic acid, which comprises both the first nucleotide sequence and the second nucleotide sequence. The coding sequences of the chimeric receptor polypeptide and the factor that redirects glucose metabolites may be in operable linkage to two distinct promoters such that the expression of the two polypeptides is controlled by different promoters. Alternatively, the coding sequences of the chimeric receptor polypeptide and the factor that redirects glucose metabolites may be in operably linkage to one promoter such that the expression of the two polypeptides is controlled by a single promoter. Suitable sequences may be inserted between the coding sequences of the two polypeptides so that two separate polypeptides can be translated from a single mRNA molecule. Such sequences, for example, IRES or ribosomal skipping site, are well known in the art. Accordingly, it is one embodiment that the nucleic acid further comprises a third nucleotide sequence located between the first nucleotide sequence and the second nucleotide sequence, wherein the third nucleotide sequence encodes a ribosomal skipping site, an internal ribosome entry site (IRES), or a promoter. Additional descriptions are provided below.

In further embodiments the nucleic acid or nucleic acid set is comprised within one or more viral vectors. The nucleic acids and the vector(s) may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the factor that redirects glucose metabolites and/or the chimeric receptor polypeptides. The synthetic linkers may contain nucleic acid sequences that correspond to a particular restriction site in the vector. The selection of expression vectors/plasmids/viral vectors would depend on the type of host cells for expression of the factor that redirects glucose metabolites and/or the chimeric receptor polypeptides, but should be suitable for integration and replication in eukaryotic cells.

A variety of promoters can be used for expression of the factor that redirects glucose metabolites and/or the chimeric receptor polypeptides described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, the human EF1-alpha promoter, or herpes simplex tk virus promoter. Additional promoters for expression of the factor that redirects glucose metabolites and/or the chimeric receptor polypeptides include any constitutively active promoter in an immune cell. Alternatively, any regulatable/inducible promoter may be used, such that its expression can be modulated within an immune cell. Suitable induction systems are known in the art, see, e.g., Kallunki et al. (Kallunki et al., Cells, 8(8): 796 (2019)).

Additionally, the vector may contain, for example, some or all of the following: a selectable marker gene, such as the neomycin gene or the kanamycin gene for selection of stable or transient transfectants in host cells; enhancer/promoter sequences from the immediate early gene of human CMV for high levels of transcription; intron sequences from the human EF1-alpha gene, transcription termination and RNA processing signals from SV40 for mRNA stability; SV40 or polyomavirus origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase or an inducible caspase such as iCasp9), and reporter gene(s) for assessing expression of the factor that redirects glucose metabolites and/or the chimeric receptor polypeptide.

In one specific embodiment, such vectors also include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Springer, C. J. (Suicide Gene Therapy: Methods and Reviews, Humana Press (2004)) and include, for example, the Herpes Simplex Virus (HSV) thymidine kinase (TK) gene, cytosine deaminase, purine nucleoside phosphorylase, nitroreductase, and caspases such as caspase 8.

Suitable vectors and methods for producing vectors containing transgenes are well known and available in the art. Examples of the preparation of vectors for expression of factors that redirect glucose metabolites and/or chimeric receptor polypeptides can be found, for example, in US 2014/0106449, herein incorporated in its entirety by reference.

Any of the vectors comprising a nucleic acid sequence that encodes a factor that redirects glucose metabolites and/or a chimeric receptor polypeptide described herein is also within the scope of the present disclosure. Such a vector, or the sequence encoding a factor that redirects glucose metabolites and/or a chimeric receptor polypeptide contained therein, may be delivered into host cells such as host immune cells by any suitable method. Methods of delivering vectors to immune cells are well known in the art and may include DNA electroporation, RNA electroporation, transfection using reagents such as liposomes, or viral transduction (e.g., retroviral transduction such as lentiviral transduction).

In some embodiments, the vectors for expression of the factors that redirect glucose metabolites and/or the chimeric receptor polypeptides are delivered to host cells by viral transduction (e.g., retroviral transduction such as lentiviral or gamma-retroviral transduction). Exemplary viral methods for delivery include, but are not limited to, recombinant retroviruses (see, e.g., WO 90/07936; WO 94/03622; WO 93/25698; WO 93/25234; WO 93/11230; WO 93/10218; and WO 91/02805; U.S. Pat. Nos. 5,219,740 and 4,777,127; GB 2,200,651; and EP 0345242), alphavirus-based vectors, and adeno-associated virus (AAV) vectors (see, e.g., WO 94/12649, WO 93/03769; WO 93/19191; WO 94/28938; WO 95/11984; and WO 95/00655). In some embodiments, the vectors for expression of the factors that redirect glucose metabolites and/or the chimeric receptor polypeptides are retroviruses. In some embodiments, the vectors for expression of the factors that redirect glucose metabolites and/or the chimeric receptor polypeptides are lentiviruses.

Examples of references describing retroviral transduction include Anderson et al., U.S. Pat. No. 5,399,346; (Mann et al., Cell, 33(1): 153-159 (1983)); U.S. Pat. Nos. 4,650,764; 4,980,289; (Markowitz et al., J Virol, 62(4): 1120-1124 (1988)); U.S. Pat. No. 5,124,263; WO 95/07358 and (Kuo et al., Blood, 82(3): 845-852 (1993)). WO 95/07358 describes high efficiency transduction of primary B lymphocytes. See also WO 2016/040441A1, all incorporated by reference herein for the purpose and subject matter referenced herein.

In examples in which the vectors encoding factors that redirect glucose metabolites and/or chimeric receptor polypeptides are introduced to the host cells using a viral vector, viral particles that are capable of infecting the immune cells and carry the vector may be produced by any method known in the art and can be found, for example in WO 91/02805A2, WO 98/09271A1, and U.S. Pat. No. 6,194,191. The viral particles are harvested from the cell culture supernatant and may be isolated and/or purified prior to contacting the viral particles with the immune cells.

In some embodiments, RNA molecules encoding any of the factors that redirect glucose metabolites and/or the chimeric receptor polypeptides as described herein may be prepared by a conventional method (e.g., in vitro transcription) and then introduced into suitable host cells, e.g., those described herein, via known methods, e.g., Rabinovich, Komarovskaya et al. (Human Gene Therapy, 17(10): 1027-1035 (2006)).

In some instances, the nucleic acid encoding a factor that redirects glucose metabolites and the nucleic acid encoding a suitable chimeric receptor polypeptide may be cloned into separate expression vectors, which may be introduced into suitable host cells concurrently or sequentially. For example, an expression vector (or an RNA molecule) for expressing the factor that redirects glucose metabolites may be introduced into host cells first and transfected host cells expressing the factor that redirects glucose metabolites may be isolated and cultured in vitro. An expression vector (or an RNA molecule) for expressing a suitable chimeric receptor polypeptide can then introduced into the host cells that express the factor that redirects glucose metabolites and transfected cells expressing both polypeptides can be isolated. In another example, expression vectors (or RNA molecules) each for expressing the factor that redirects glucose metabolites and the chimeric receptor polypeptide can be introduced into host cells simultaneously and transfected host cells expressing both polypeptides can be isolated via routine methodology.

In other instances, the nucleic acid encoding the factor that redirects glucose metabolites and the nucleic acid encoding the chimeric receptor polypeptide may be cloned into the same expression vector. Polynucleotides (including vectors in which such polynucleotides are operably linked to at least one regulatory element) for expression of the chimeric receptor polypeptide and factor that redirects glucose metabolites are also within the scope of the present disclosure. Non-limiting examples of useful vectors of the disclosure include viral vectors such as, e.g., retroviral vectors including gamma retroviral vectors and lentiviral vectors, and adeno-associated virus vectors (AAV vectors).

In some instances, the nucleic acid(s) encoding the factor that redirects glucose metabolites and/or the chimeric receptor polypeptide may be delivered into host cells via transposon (e.g., piggybac). In some instances, the encoding nucleic acid(s) may be delivered into host cells via gene editing, for example, by CRISPR, TALEN, zinc-finger nuclease (ZFN), or meganucleases.

In some instances, the nucleic acid described herein may comprise two coding sequences, one encoding a chimeric receptor polypeptide as described herein, and the other encoding a polypeptide capable of redirecting glucose out of the glycolysis pathway (i.e., a factor that redirects glucose metabolites). In some instances, recombinant TCRs comprising TCR chains (e.g., α and β TCR chains) are separated by a self-cleaving 2A peptide such as P2A or T2A. The nucleic acid comprising the two coding sequences described herein may be configured such that the polypeptides encoded by the two coding sequences can be expressed as independent (and physically separate) polypeptides. To achieve this goal, the nucleic acid described herein may contain a third nucleotide sequence located between the first and second coding sequences. This third nucleotide sequence may, for example, encode a ribosomal skipping site. A ribosomal skipping site is a sequence that impairs normal peptide bond formation. This mechanism results in the translation of additional open reading frames from one messenger RNA. This third nucleotide sequence may, for example, encode a P2A, T2A, or F2A peptide (see, for example, Kim, Lee et al. (PLoS One, 6(4): e18556 (2011)). See Table 16 below.

TABLE 16
Exemplary Ribosomal Skipping Peptides

| Ribosomal | | |
|---|---|---|
| Skipping | | |
| Site | Sequence | SEQ ID NO |
| P2A | ATNFSLLKQAGDVEENPGP | SEQ ID NO. 67 |
| T2A | EGRGSLLTCGDVEENPGP | SEQ ID NO. 82 |
| E2A | QCTNYALLKLAGDVESNPGP | SEQ ID NO. 83 |
| F2A | AVKQTLNFDLLKLAGDVESNPGP | SEQ ID NO. 84 |

In another embodiment, the third nucleotide sequence may encode an internal ribosome entry site (IRES). An IRES is an RNA element that allows translation initiation in an end-independent manner, also permitting the translation of additional open reading frames from one messenger RNA. Alternatively, the third nucleotide sequence may encode a promoter controlling the expression of the second polypeptide. The third nucleotide sequence may also encode more than one ribosomal skipping sequence, IRES sequence, additional promoter sequence, or a combination thereof.

The nucleic acid may also include additional coding sequences (including, but not limited to, fourth and fifth coding sequences) and may be configured such that the polypeptides encoded by the additional coding sequences are expressed as further independent and physically separate polypeptides. To this end, the additional coding sequences may be separated from other coding sequences by one or more nucleotide sequences encoding one or more ribosomal skipping sequences, IRES sequences, or additional promoter sequences.

An exemplar IRES sequence is provided below (SEQ ID NO: 85):

GAGGGCCCGGAAACCTGGCCCTGTCTTCTTGACGAGCATTCCTAGGGGTC
TTTCCCCTCTCGCCAAAGGAATGCAAGGTCTGTTGAATGTCGTGAAGGAA
GCAGTTCCTCTGGAAGCTTCTTGAAGACAAACAACGTCTGTAGCGACCCT
TTGCAGGCAGCGGAACCCCCCACCTGGCGACAGGTGCCTCTGCGGCCAAA
AGCCACGTGTATAAGATACACCTGCAAAGGCGGCACAACCCCAGTGCCAC
GTTGTGAGTTGGATAGTTGTGGAAAGAGTCAAATGGCTCTCCTCAAGCGT
ATTCAACAAGGGGCTGAAGGATGCCCAGAAGGTACCCCATTGTATGGGAT
CTGATCTGGGGCCTCGGTGCACATGCTTTACATGTGTTTAGTCGAGGTTA
AAAAAACGTCTAGGCCCCCCGAACCACGGGGACGTGGTTTTCCTTTGAAA
AACACGATGATAA

In some examples, the nucleic acid (e.g., an expression vector or an RNA molecule as described herein) may comprise coding sequences for both the factor that redirects glucose metabolites (e.g., those described herein) and a suitable chimeric receptor polypeptide, the two coding sequences, in any order, being separated by a third nucleotide sequence coding for a P2A peptide (e.g., SEQ ID NO: 67). As a result, two separate polypeptides, the factor that redirects glucose metabolites and the chimeric receptor, can be produced from such a nucleic acid, wherein the P2A portion SEQ ID NO: 67) is linked to the upstream polypeptide (encoded by the upstream coding sequence) and residue P from the P2A peptide is linked to the downstream polypeptide (encoded by the downstream coding sequence). In some examples, the chimeric receptor polypeptide is the upstream one and the factor that redirects glucose metabolites is the downstream one. In other examples, the factor that redirects glucose metabolites is the upstream one and the chimeric receptor polypeptide is the downstream one. In some embodiments, the nucleic acid (e.g., an expression vector or an RNA molecule as described herein) may comprise coding sequences for both the factor that redirects glucose metabolites (e.g., those described herein) and a suitable TCR, ACTR or CAR polypeptide, the two coding sequences, in any order, being separated by a third nucleotide sequence coding for a P2A peptide (e.g., SEQ ID NO: 67). As a result, two separate polypeptides, the factor that redirects glucose metabolites and the TCR, ACTR or CAR) can be produced from such a nucleic acid, wherein the P2A portion SEQ ID NO: 67 is linked to the upstream polypeptide (encoded by the upstream coding sequence) and residue P from the P2A peptide is linked to the downstream polypeptide (encoded by the downstream coding sequence). In some embodiments, the TCR, ACTR or CAR polypeptide is the upstream one and the factor that redirects glucose metabolites is the downstream one. In other embodiments, the factor that redirects glucose metabolites is the upstream one and the TCR, ACTR or CAR polypeptide is the downstream one.

In some examples, the nucleic acid described above may further encode a linker (e.g., a GSG linker) between two segments of the encoded sequences, for example, between the upstream polypeptide and the P2A peptide.

In specific examples, the nucleic acid described herein is configured such that it expresses two separate polypeptides in the host cell to which the nucleic acid is transfected: (i) the first polypeptide that contains, from the N-terminus to the C-terminus, a suitable CAR (e.g., enlisted in Tables 11-14 or SEQ ID NO: 78-SEQ ID NO: 79), a peptide linker (e.g., the GSG linker), and the ATNFSLLKQAGDVEENPG (SEQ ID NO: 67) segment derived from the P2A peptide; and (ii) a second polypeptide that contains, from the N-terminus to the C-terminus, the P residue derived from the P2A peptide and the factor that redirects glucose metabolites (e.g., any of SEQ ID NOs: SEQ ID NO: 68-SEQ ID NO: 74).

In specific examples, the nucleic acid described herein is configured such that it expresses two separate polypeptides in the host cell to which the nucleic acid is transfected: (i) the first polypeptide that contains, from the N-terminus to the C-terminus, a suitable ACTR (see Table 7, a peptide linker (e.g., the GSG linker), and the ATNFSLLKQAGDVEENPG (SEQ ID NO: 67) segment derived from the P2A peptide; and (ii) a second polypeptide that contains, from the N-terminus to the C-terminus, the P residue derived from the P2A peptide and the factor that redirects glucose metabolites (e.g., any of SEQ ID NO: 68-SEQ ID NO: 74). In some instances, additional polypeptides of interest may also be introduced into the host immune cells.

Following introduction into the host cells a vector encoding any of the factors that redirect glucose metabolites and/or the chimeric receptor polypeptides provided herein, or the nucleic acid encoding the chimeric receptor polypeptide and/or factor that redirects glucose metabolites (e.g., an RNA molecule), the cells may be cultured under conditions that allow for expression of the factors that redirect glucose metabolites and/or the chimeric receptor polypeptide. In examples in which the nucleic acid encoding the factors that redirect glucose metabolites and/or the chimeric receptor polypeptide is regulated by a regulatable promoter, the host cells may be cultured in conditions wherein the regulatable promoter is activated. In some embodiments, the promoter is an inducible promoter and the immune cells are cultured in the presence of the inducing molecule or in conditions in which the inducing molecule is produced. Determining whether the factor that redirects glucose metabolites and/or the chimeric receptor polypeptide is expressed will be evident to one of skill in the art and may be assessed by any known method, for example, detection of the factor that redirects glucose metabolites and/or the chimeric receptor polypeptide-encoding mRNA by quantitative reverse transcriptase PCR (qRT-PCR) or detection of the factor that redirects glucose metabolites and/or the chimeric receptor polypeptide protein by methods including Western blotting, fluorescence microscopy, and flow cytometry.

Alternatively, expression of the chimeric receptor polypeptide may take place in vivo after the immune cells are administered to a subject. As used herein, the term “subject” refers to any mammal such as a human, monkey, mouse, rabbit, or domestic mammal. For example, the subject may be a primate. In a preferred embodiment, the subject is human.

Alternatively, expression of a factor that redirects glucose metabolites and/or a chimeric receptor polypeptide in any of the immune cells disclosed herein can be achieved by introducing RNA molecules encoding the factors that redirect glucose metabolites and/or the chimeric receptor polypeptides. Such RNA molecules can be prepared by in vitro transcription or by chemical synthesis. The RNA molecules can then be introduced into suitable host cells such as immune cells (e.g., T or NK cells) by, e.g., electroporation. For example, RNA molecules can be synthesized and introduced into host immune cells following the methods described in Rabinovich, Komarovskaya et al. (Human Gene Therapy, 17(10): 1027-1035 (2006)) and WO 2013/040557.

In certain embodiments, a vector(s) or RNA molecule(s) comprising the factor that redirects glucose metabolites and/or the chimeric receptor polypeptide may be introduced to the host cells or immune cells in vivo. As a non-limiting example, this may be accomplished by administering a vector or RNA molecule encoding one or more factors that redirect glucose metabolites and/or one or more chimeric receptor polypeptides described herein directly to the subject (e.g., through intravenous administration), producing host cells comprising factors that redirect glucose metabolites and/or chimeric receptor polypeptides in vivo.

Methods for preparing host cells expressing any of the factors that redirect glucose metabolites and/or the chimeric receptor polypeptides described herein may also comprise activating the host cells ex vivo. Activating a host cell means stimulating a host cell into an activated state in which the cell may be able to perform effector functions. Methods of activating a host cell will depend on the type of host cell used for expression of the factors that redirect glucose metabolites and/or chimeric receptor polypeptides. For example, T cells may be activated ex vivo in the presence of one or more molecules including, but not limited to: an anti-CD3 antibody, an anti-CD28 antibody, IL-2, phytohemoagglutinin, engineered artificial stimulatory cells or particles, or a combination thereof. The engineered artificial stimulatory cells may be artificial antigen-presenting cells as known in the art. See, e.g., Neal, Bailey et al. (J Immunol Res Ther, 2(1): 68-79 (2017)) and Turtle and Riddell (Cancer journal (Sudbury, Mass.), 16(4): 374-381 (2010)), the relevant disclosures of each of which are hereby incorporated by reference for the purpose and subject matter referenced herein.

In other examples, NK cells may be activated ex vivo in the presence of one or more molecules such as a 4-1BB ligand, an anti-4-1BB antibody, IL-2, IL-15, an anti-IL-15 receptor antibody, IL12, IL-21, K562 cells, and/or engineered artificial stimulatory cells or particles. In some embodiments, the host cells expressing any of the factors that redirect glucose metabolites and/or the chimeric receptor polypeptides (ACTR-/CAR-/TCR- and/or factor that redirect glucose metabolites-expressing cells) described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

Methods for preparing host cells expressing any of the factors that redirect glucose metabolites and/or the chimeric receptor polypeptides described herein may comprise expanding the host cells ex vivo. Expanding host cells may involve any method that results in an increase in the number of cells expressing factors that redirect glucose metabolites and/or chimeric receptor polypeptides, for example, allowing the host cells to proliferate or stimulating the host cells to proliferate. Methods for stimulating expansion of host cells will depend on the type of host cell used for expression of the factors that redirect glucose metabolites and/or the chimeric receptor polypeptides and will be evident to one of skill in the art. In some embodiments, the host cells expressing any of the factors that redirect glucose metabolites and/or the chimeric receptor polypeptides described herein are expanded ex vivo prior to administration to a subject.

In some embodiments, the host cells expressing the factors that redirect glucose metabolites and/or the chimeric receptor polypeptides are expanded and activated ex vivo prior to administration of the cells to the subject. Host cell activation and expansion may be used to allow integration of a viral vector into the genome and expression of the gene encoding a factor that redirects glucose metabolites and/or a chimeric receptor polypeptide as described herein. If mRNA electroporation is used, no activation and/or expansion may be required, although electroporation may be more effective when performed on activated cells. In some instances, a factor that redirects glucose metabolites and/or a chimeric receptor polypeptide is transiently expressed in a suitable host cell (e.g., for 3-5 days). Transient expression may be advantageous if there is a potential toxicity and should be helpful in initial phases of clinical testing for possible side effects. Any of the host cells expressing the factors that redirect glucose metabolites and/or the chimeric receptor polypeptides may be mixed with a pharmaceutically acceptable carrier to form a pharmaceutical composition, which is also within the scope of the present disclosure. Therefore, a pharmaceutical composition, comprising a genetically engineered immune cell of the invention is another embodiment. The genetically engineered immune cells are preferably mixed with a pharmaceutically acceptable carrier.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

The pharmaceutical compositions of the disclosure may also contain one or more additional active compounds as necessary for the particular indication being treated, preferably those with complementary activities that do not adversely affect each other. Non-limiting examples of possible additional active compounds include, e.g., IL-2 as well as various agents known in the field and listed in the discussion of combination treatments, below.

### IV. Immunotherapy Using the Genetically Engineered Hematopoietic Cells Described Herein

The genetically engineered hematopoietic cells (e.g., hematopoietic stem cells, immune cells, such as NK cells or T cells) disclosed herein may be used in immunotherapy against various disorders, for example, cancer, infectious diseases, and autoimmune diseases. Accordingly, another embodiment of the present invention is a method for inhibiting cells expressing a target antigen in a subject, the method comprising administering to a subject in need thereof a population of the genetically engineered immune cells set forth herein or a pharmaceutical composition comprising a population of the genetically engineered immune cells set forth herein.

### A. Combined Immunotherapy of Genetically Engineered Hematopoietic Cells Expressing ACTR Polypeptides and Fc-Containing Therapeutic Agents

The exemplary ACTR polypeptides of the present disclosure confer antibody-dependent cell cytotoxicity (ADCC) capacity to T lymphocytes and enhance ADCC in NK cells. When the receptor is engaged by an antibody bound to cells, it triggers T-cell activation, sustained proliferation and specific cytotoxicity against the bound cells.

The degree of affinity of CD16 for the Fc portion of Ig is a critical determinant of ADCC and thus to clinical responses to antibody immunotherapy. The CD16 with the V158 polymorphism which has a higher binding affinity for Ig and mediates superior ADCC relative to CD16 with the F158 polymorphism was selected as an example. Although the F158 receptor has lower potency than the V158 receptor in induction of T cell proliferation and ADCC, the F158 receptor may have lower in vivo toxicity than the V158 receptor making it useful in some clinical contexts.

The factors that redirect glucose metabolites to be co-expressed with an ACTR polypeptides in immune cells would facilitate cell-based immune therapy such as T-cell therapy or NK-cell therapy by allowing the cells to grow and/or function effectively in a low glucose, low amino acid, low pH, and/or hypoxic environment. Antibody-directed cytotoxicity could be stopped whenever required by simple withdrawal of antibody administration. Clinical safety can be further enhanced by using mRNA electroporation to express the factors that redirect glucose metabolites and/or the ACTR polypeptides transiently, to limit any potential autoimmune reactivity.

Thus, in one embodiment, the disclosure provides a method for enhancing efficacy of an antibody-based immunotherapy of a cancer in a subject in need thereof, which subject is being treated with an Fc-containing therapeutic agent such as a therapeutic antibody, which can bind to antigen-expressing cells. The Fc-containing therapeutic agent contains an Fc portion, for example, a human or humanized Fc portion, which can be recognized and bound by the Fc-binding portion (e.g., the extracellular domain of human CD16A) of the ACTR expressed on the engineered immune cells. Exemplary ACTR constructs are provided in Table 10 above.

The methods described herein may comprise introducing into the subject a therapeutically effective amount an antibody and a therapeutically effective amount of the genetically engineered host cells such as hematopoietic cells, for example, immune cells (e.g., T or NK cells), which co-express a factor that redirects glucose metabolites and an ACTR polypeptide of the disclosure. The subject (e.g., a human patient such as a human cancer patient) has been treated or is being treating with an Fc-containing therapeutic agent specific to a target antigen. A target antigen may be any molecule that is associated with a disease or condition, including, but are not limited to, tumor antigens, pathogenic antigens (e.g., bacterial, fungal or viral), or antigens present on diseased cells, such as those described herein.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

As used herein the term “therapeutically effective” applied to dose or amount refers to that quantity of a compound or pharmaceutical composition that is sufficient to result in a desired activity upon administration to a subject in need thereof. Note that when a combination of active ingredients is administered (e.g., a first pharmaceutical composition comprising an antibody, and a second pharmaceutical composition comprising a population of T or NK cells that express a factor that redirects glucose metabolites and/or an antibody-coupled T-cell receptor (ACTR) construct, the effective amount of the combination may or may not include amounts of each ingredient that would have been effective if administered individually. Within the context of the present disclosure, the term “therapeutically effective” refers to that quantity of a compound or pharmaceutical composition that is sufficient to delay the manifestation, arrest the progression, relieve or alleviate at least one symptom of a disorder treated by the methods of the present disclosure.

Host cells (e.g., hematopoietic cells, for example, immune cells such as T and NK cells) expressing factors that redirect glucose metabolites and ACTR or CAR polypeptides described herein are useful for enhancing ADCC in a subject and/or for enhancing the efficacy of an antibody-based immunotherapy and/or for enhancing growth and/or proliferation of immune cells in a low-glucose environment. In some embodiments, the subject is a mammal, such as a human, monkey, mouse, rabbit, or domestic mammal. In some embodiments, the subject is a human. In some embodiments, the subject is a human cancer patient. In some embodiments, the subject has been treated or is being treated with any of the therapeutic antibodies described herein.

To practice the method described herein, an effective amount of the host cells, for example, immune cells (e.g., NK cells and/or T lymphocytes) expressing any of the factors that redirect glucose metabolites and the ACTR polypeptides described herein and an effective amount of an antibody, or compositions thereof may be administered to a subject in need of the treatment via a suitable route, such as intravenous administration. As used herein, an effective amount refers to the amount of the respective agent (e.g., the NK cells and/or T lymphocytes expressing factors that redirect glucose metabolites, ACTR polypeptides, antibodies, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject in need of treatment is a human. In some embodiments, the subject in need of treatment is a human cancer patient. In some embodiments, the subject in need of treatment suffers from one or more pathogenic infections (e.g., viral, bacterial, and/or fungal infections).

The methods of the disclosure may be used for treatment of any cancer or any pathogen. Specific non-limiting examples of cancers which can be treated by the methods of the disclosure include, for example, lymphoma, breast cancer, gastric cancer, neuroblastoma, osteosarcoma, lung cancer, skin cancer, prostate cancer, colorectal cancer, renal cell carcinoma, ovarian cancer, rhabdomyosarcoma, leukemia, mesothelioma, pancreatic cancer, head and neck cancer, retinoblastoma, glioma, glioblastoma, thyroid cancer, hepatocellular cancer, esophageal cancer, and cervical cancer. In certain embodiments, the cancer may be a solid tumor.

The methods of this disclosure may also be used for treating infectious diseases, which may be caused by bacterial infection, viral infection, or fungus infection. In such instances, the genetically engineered immune cells can be co-used with an Fc-containing therapeutic agent (e.g., an antibody) that targets a pathogenic antigen (e.g., an antigen associated with the bacterium, virus, or fungus that causes the infection). Specific non-limiting examples of pathogenic antigens include, but are not limited to, bacterial, viral, and/or fungal antigens. Some examples are provided below: influenza virus neuraminidase, hemagglutinin, or M2 protein, human respiratory syncytial virus (RSV) F glycoprotein or G glycoprotein, herpes simplex virus glycoprotein gB, gC, gD, or gE, Chlamydia MOMP or PorB protein, Dengue virus core protein, matrix protein, or glycoprotein E, measles virus hemagglutinin, herpes simplex virus type 2 glycoprotein gB, poliovirus I VP1, envelope glycoproteins of HIV 1, hepatitis B core antigen or surface antigen, diptheria toxin, Streptococcus 24M epitope, Gonococcal pilin, pseudorabies virus g50 (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, or human hepatitis C virus glycoprotein E1 or E2.

In some embodiments, the immune cells are administered to a subject in an amount effective in enhancing ADCC activity by least 20% and/or by at least 2-fold, e.g., enhancing ADCC by 50%, 80%, 100%, 2-fold, 5-fold, 10-fold, 20-fold, 50-fold, 100-fold, or more.

The immune cells are co-administered with an Fc-containing therapeutic agent such as a therapeutic antibody in order to target cells expressing the antigen to which the Fc-containing therapeutic agent binds. In some embodiments, more than one Fc-containing therapeutic agents, such as more than one antibody can be co-used with the immune cells. Antibody-based immunotherapy may be used to treat, alleviate, or reduce the symptoms of any disease or disorder for which the immunotherapy is considered useful in a subject.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof which comprise an Fc region, mutants thereof, fusion proteins comprising an antibody portion, humanized antibodies, chimeric antibodies, diabodies, single domain antibodies (e.g., nanobodies), linear antibodies, multispecific antibodies (e.g., bispecific antibodies) and any other modified configuration of the immunoglobulin molecule that comprises an antigen recognition site of the required specificity and an Fc region, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The antibody for use in the present disclosure contains an Fc region recognizable by the co-used ACTR- and/or factor that redirects glucose metabolites-expressing immune cells. The Fc region may be a human or humanized Fc region.

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. The antibodies used herein may be glycosylated (e.g., fucosylated) or afucoslylated. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as a human. In some embodiments, amino acid modifications can be made in the variable region and/or the constant region.

The hematopoietic cells, for example, immune cells (e.g., T and/or NK cells) or HSCs expressing any of the factors that redirect glucose metabolites and/or the ACTR polypeptides disclosed herein may be administered to a subject who has been treated or is being treated with an Fc-containing antibody. For example, the immune cells may be administered to a human subject simultaneously with an antibody. Alternatively, the immune cells may be administered to a human subject during the course of an antibody-based immunotherapy. In some examples, the immune cells and an antibody can be administered to a human subject at least 4 hours apart, e.g., at least 12 hours apart, at least 1 day apart, at least 3 days apart, at least one week apart, at least two weeks apart, or at least one month apart.

In some embodiments, the antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen.

In some embodiments, an antibody as described herein has a suitable binding affinity for the target antigen (e.g., any one of the targets described herein) or antigenic epitopes thereof. The antibodies for use in the immune therapy methods described herein may bind to (e.g., specifically bind to) a target antigen of interest, or a specific region or an antigenic epitope therein. Table 4 above lists exemplary target antigens of interest and exemplary antibodies specific to such.

### B. Immunotherapy of Genetically Engineered Hematopoietic Cells Expressing CAR Polypeptides

The genetically engineered hematopoietic cells (e.g., hematopoietic stem cells, immune cells, such as T or NK cells) described herein, co-expressing a factor that redirects glucose metabolites and a CAR polypeptide can be used in immune therapy such as T-cell therapy (both αβ and γδ T cells) or NK-cell therapy for inhibiting diseased cells expressing an antigen to which the CAR polypeptide targets, directly or indirectly (e.g., via a therapeutic agent conjugated to a tag to which the CAR polypeptide binds). The factor that redirects glucose metabolites co-expressed with a CAR polypeptide in immune cells would facilitate the cell-based immune therapy by allowing the cells to grow and/or function effectively in a low glucose, low amino acid, low pH, and/or a hypoxic environment, for example, in a tumor microenvironment. Clinical safety may be further enhanced by using mRNA electroporation to express the factors that redirect glucose metabolites and/or the CAR polypeptides transiently, to limit any potential non-tumor specific reactivity.

The methods described herein may comprise introducing into the subject a therapeutically effective amount of genetically engineered host cells such as hematopoietic cells, for example, immune cells (e.g., αβ T, γδ T or NK cells), which co-express a factor that redirects glucose metabolites and a CAR polypeptide of the disclosure (see exemplary examples in Table 11-Table 14). The subject (e.g., a human patient such as a human cancer patient) may additionally have been treated or is being treated with an anti-cancer or anti-infection therapy including, but not limited to, an anti-cancer therapeutic agent or anti-infection agent. The CAR has an antigen-binding domain that may bind any target antigen. Such a target antigen may be any molecule that is associated with a disease or condition, including, but are not limited to, tumor antigens, pathogenic antigens (e.g., bacterial, fungal, or viral), or antigens present on diseased cells, such as those described herein. In some embodiments, the target antigen binding domain targets a native tumor antigen protein. In other embodiments, the target antigen binding domain targets a variant (e.g., mutation) of a tumor antigen protein. Some examples include EGFRvIII scFv recognizes the tumor specific variant of EGFR (Wang, Jiang et al., Cancer Lett, 472: 175-180 (2020)).

Host cells (e.g., hematopoietic cells, for example, immune cells such as T and NK cells) expressing factors that redirect glucose metabolites and CAR polypeptides described herein are useful for inhibiting cells expressing a target antigen and/or for enhancing growth and/or proliferation of immune cells in a low-glucose environment, a low amino acid environment, a low pH environment, and/or a hypoxic environment, for example, in a tumor microenvironment. In some embodiments, the subject is a mammal, such as a human, monkey, mouse, rabbit, or domestic mammal. In some embodiments, the subject is a human. In some embodiments, the subject is a human cancer patient. In some embodiments, the subject has additionally been treated or is being treated with any of the therapeutic antibodies described herein.

To practice the method described herein, an effective amount of the hematopoietic cells, for example, immune cells (NK and/or T cells) expressing any of the factors that redirect glucose metabolites and the CAR polypeptides described herein, or compositions thereof may be administered to a subject in need of the treatment via a suitable route, such as intravenous, subcutaneous and intradermal administration. As used herein, an effective amount refers to the amount of the respective agent (e.g., the NK and/or T cells expressing factors that redirect glucose metabolites, CAR polypeptides, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject in need of treatment is a human. In some embodiments, the subject in need of treatment is a human cancer patient. In some embodiments, the subject in need of treatment suffers from one or more pathogenic infections (e.g., viral, bacterial, and/or fungal infections).

The methods of the disclosure may be used for treatment of any cancer or any pathogen. Specific non-limiting examples of cancers which can be treated by the methods of the disclosure include, for example, lymphoma, breast cancer, gastric cancer, neuroblastoma, osteosarcoma, lung cancer, skin cancer, prostate cancer, colorectal cancer, renal cell carcinoma, ovarian cancer, rhabdomyosarcoma, leukemia, mesothelioma, pancreatic cancer, head and neck cancer, retinoblastoma, glioma, glioblastoma, thyroid cancer, hepatocellular cancer, esophageal cancer, and cervical cancer. In certain embodiments, the cancer may be a solid tumor. In certain embodiments, the cancer may be a liquid tumor. Accordingly, a preferred embodiment is a method for inhibiting cells expressing a target antigen in a subject, wherein the subject is a human patient suffering from a cancer and the target antigen is a tumor antigen; wherein the cancer is selected from the group consisting of carcinoma, lymphoma, sarcoma, blastoma, and leukemia, preferably wherein the cancer is selected from the group consisting of a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, liver cancer, and thyroid cancer; or the cancer of B-cell origin is selected from the group consisting of B-lineage acute lymphoblastic leukemia, B-cell chronic lymphocytic leukemia, and B-cell non-Hodgkin's lymphoma.

The methods of this disclosure may also be used for treating infectious diseases, which may be caused by bacterial infection, viral infection, or fungal infection. In such instances, genetically engineered immune cells expressing a CAR polypeptide specific to a pathogenic antigen, (e.g., an antigen associated with the bacterium, virus, or fungus that causes the infection) can be used to eliminate infected cells. Specific non-limiting examples of pathogenic antigens include, but are not limited to, bacterial, viral, and/or fungal antigens.

In some embodiments, the immune cells are administered to a subject in an amount effective in inhibiting cells expressing the target antigen by least 20% and/or by at least 2-fold, e.g., inhibiting cells expressing the target antigen by 50%, 80%, 100%, 2-fold, 5-fold, 10-fold, 20-fold, 50-fold, 100-fold, or more.

Additional therapeutic agents (e.g., antibody-based immunotherapeutic agents) may be used to treat, alleviate, or reduce the symptoms of any disease or disorder for which the therapeutic agent is considered useful in a subject.

The efficacy of the cell-based immunotherapy as described herein may be assessed by any method known in the art and would be evident to a skilled medical professional. For example, the efficacy of the cell-based immunotherapy may be assessed by survival of the subject or tumor or cancer burden in the subject or tissue or sample thereof. In some embodiments, the immune cells are administered to a subject in need of the treatment in an amount effective in enhancing the efficacy of an cell-based immunotherapy by at least 20% and/or by at least 2-fold, e.g., enhancing the efficacy of an antibody-based immunotherapy by 50%, 80%, 100%, 2-fold, 5-fold, 10-fold, 20-fold, 50-fold, 100-fold or more, as compared to the efficacy in the absence of the immune cells expressing the factors that redirect glucose metabolites and/or the CAR polypeptide.

In any of the compositions or methods described herein, the immune cells (e.g., NK and/or T cells) may be autologous to the subject, i.e., the immune cells may be obtained from the subject in need of the treatment, genetically engineered for expression of the factors that redirect glucose metabolites and/or the CAR polypeptides, and then administered to the same subject. In one specific embodiment, prior to re-introduction into the subject, the autologous immune cells (e.g., T or NK cells) are activated and/or expanded ex vivo. Administration of autologous cells to a subject may result in reduced rejection of the host cells as compared to administration of non-autologous cells.

Alternatively, the host cells are allogeneic cells, i.e., the cells are obtained from a first subject, genetically engineered for expression of the factors that redirect glucose metabolites and/or the CAR polypeptide and administered to a second subject that is different from the first subject but of the same species. For example, allogeneic immune cells may be derived from a human donor and administered to a human recipient who is different from the donor. In a specific embodiment, the T cells are allogeneic T cells in which the expression of the endogenous T cell receptor has been inhibited or eliminated. In one specific embodiment, prior to introduction into the subject, the allogeneic T cells are activated and/or expanded ex vivo. T lymphocytes can be activated by any method known in the art, e.g., in the presence of anti-CD3/CD28, IL-2, IL-15, phytohemoagglutinin, engineered artificial stimulatory cells or particles, or a combination thereof. In certain aspects, the starting population of NK or T cells is obtained from isolating mononuclear cells using ficoll-paque density gradient. In some aspects, the method further comprises depleting the mononuclear cells of CD3, CD14, and/or CD19 cells to obtain the starting population of NK cells. In some aspects, the method further comprises depleting the mononuclear cells CD3, CD14, and CD19 cells to obtain the starting population of NK cells. In particular aspects, depleting comprises performing magnetic sorting. In other aspects, NK cells could be positively selected using sorting, magnetic bead selection or other methods to obtain the starting populations of NK cells.

Additionally, immune cells such as NK cells are derived from cord blood stem cells or induced pluripotent stem cells (iPSCs) providing from “off-the shelf” source for immunotherapy (Li et al., Cell Stem Cell, 23(2): 181-192.e185 (2018); Liu et al., Leukemia, 32(2): 520-531 (2018); Morgan et al., Front Immunol, 11: 1965 (2020); Wrona, Borowiec et al., Int J Mol Sci, 22(11): (2021)). In particular embodiments, the starting population of NK cells is obtained from cord blood. In other embodiments, the cord blood has previously been frozen. In some embodiments, cells are derived from cell lines (e.g., NK-92 and Vγ9V52 T cell).

NK and T cells (αβ T or γδ T cells) can be activated by any method known in the art, e.g., in the presence of one or more agents selected from the group consisting of CD137 ligand protein, CD137 antibody, IL-15, IL-15 receptor antibody, IL-2, IL-12, IL-21, and cells from the K562 cell line, and/or engineered artificial stimulatory cells or particles. See, e.g., U.S. Pat. Nos. 7,435,596 and 8,026,097 for the description of useful methods for expanding NK cells. For example, NK cells used in the compositions or methods disclosed herein may be preferentially expanded by exposure to cells that lack or poorly express major histocompatibility complex I and/or II molecules and which have been genetically modified to express membrane bound IL-15 and 4-1BB ligand (CD137L). Such cell lines include, but are not necessarily limited to, K562 [ATCC, CCL 243; (Lozzio and Lozzio, Blood, 45(3): 321-334 (1975); Klein et al., Int J Cancer, 18(4): 421-431 (1976))], and the Wilms tumor cell line HFWT (Fehniger and Caligiuri, Int Rev Immunol, 20(3-4): 503-534 (2001); Harada et al., Exp Hematol, 32(7): 614-621 (2004)), the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastasized neuroblastoma cell line TNB 1 (Harada et al., Jpn J Cancer Res, 93(3): 313-319 (2002)). Preferably the cell line used lacks or poorly expresses both MHC I and II molecules, such as the K562 and HFWT cell lines. A solid support may be used instead of a cell line. Such support should preferably have attached on its surface at least one molecule capable of binding to NK cells and inducing a primary activation event and/or a proliferative response or capable of binding a molecule having such an affect thereby acting as a scaffold. The support may have attached to its surface the CD137 ligand protein, a CD137 antibody, the IL-15 protein or an IL-15 receptor antibody. Preferably, the support will have IL-15 receptor antibody and CD137 antibody bound on its surface.

In one embodiment of the described compositions or methods, introduction (or re-introduction) of T lymphocytes, NK cells, or T lymphocytes and NK cells to the subject is followed by administering to the subject a therapeutically effective amount of IL-2.

In additional aspects, the method further comprises cryopreserving the population of engineered NK or T cells. In some instances, the engineered NK or γδ T cells are cryopreserved. Further provided herein is a genetically engineered population of cryopreserved NK or T cells.

In accordance with the present disclosure, patients can be treated by infusing therapeutically effective doses of immune cells such as T or NK cells comprising a factor that redirects glucose metabolites and/or a CAR polypeptide of the disclosure in the range of about 10⁵ to 10¹⁰ or more cells per kilogram of body weight (cells/Kg). The infusion can be repeated as often and as many times as the patient can tolerate until the desired response is achieved. The appropriate infusion dose and schedule will vary from patient to patient, but can be determined by the treating physician for a particular patient. Typically, initial doses of approximately 10⁶ cells/kg will be infused, escalating to 10⁸ or more cells/kg. IL-2 can be co-administered to expand infused cells. The amount of IL-2 can about 1-5×10⁶ international units per square meter of body surface.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The efficacy of the compositions or methods described herein may be assessed by any method known in the art and would be evident to a skilled medical professional. For example, the efficacy of the compositions or methods described herein may be assessed by survival of the subject or cancer or pathogen burden in the subject or tissue or sample thereof. In some embodiments, the compositions and methods described herein may be assessed based on the safety or toxicity of the therapy (e.g., administration of the immune cells expressing the factors that redirect glucose metabolites and the CAR polypeptides) in the subject, for example, by the overall health of the subject and/or the presence of adverse events or severe adverse events.

### C. Other Immunotherapies

In some embodiments, the genetically engineered immune cells, expressing one or more of the factors that redirect glucose metabolites (e.g., PKM2, GFPT1, or TIGAR), may be derived from natural immune cells specific to diseased cells (e.g., cancer cells or pathogen infected cells). Such genetically engineered immune cells (e.g., tumor-infiltrating lymphocytes or TILs) may not co-express any chimeric receptor polypeptide and can be used to destroy the target disease cells, e.g., cancer cells. The genetically engineered TILs, expressing one or more factors that redirect glucose metabolites but not chimeric receptors, may be co-used with a bispecific antibody capable of binding to the target tumor cells and the TILs (BiTE).

In some embodiments, the genetically engineered immune cells, expressing one or more of the factors that redirect glucose metabolites (e.g., PKM2, GFPT1, or TIGAR), may be T_{reg} cells. Such T_{reg} cells may co-express a chimeric receptor polypeptide as disclosed herein. Alternatively, the T_{reg} cells may not co-express any chimeric receptor polypeptide and can be used for the intended therapy.

Some embodiments, provides a composition comprising an effective amount of the engineered NK or T cells of the embodiments for use in the treatment of a disease or disorder in a subject. Also provided herein is the use of a composition comprising an effective amount of the engineered NK or T cells of the embodiments for the treatment of an immune-related disorder in a subject. A further embodiment provides a method of treating an immune-related disorder in a subject comprising administering an effective amount of engineered NK or γδ T cells of the embodiments to the subject. In exemplary embodiments, the method does not comprise performing HLA matching. In particular embodiments, the NK or γδ T cells are KIR-ligand mismatched between the subject and donor. In further specific embodiments, the method does not comprise performing HLA matching. In particular embodiments, the absence of HLA matching does not result in graft versus host disease or toxicity.

### D. Immunotherapy of Genetically Engineered Hematopoietic Cells Expressing TCR Polypeptides

The genetically engineered hematopoietic cells (e.g., hematopoietic stem cells, immune cells, such as T or NKT cells, iPSCs) described herein, co-expressing a factor that redirects glucose metabolites and a TCR polypeptide can be used in immune therapy such as T-cell therapy (both αβ and γδ T cells) or NKT-cell therapy for inhibiting diseased cells expressing an antigen to which the TCR polypeptide targets, directly (e.g., via recognition of a specific peptide-MHC). The factor that redirects glucose metabolites co-expressed with a TCR polypeptide in immune cells would facilitate the cell-based immune therapy by allowing the cells to grow and/or function effectively in a low glucose, low amino acid, low pH, and/or a hypoxic environment, for example, in a tumor microenvironment. Clinical safety may be further enhanced by using mRNA electroporation to express the factors that redirect glucose metabolites and/or the TCR polypeptides transiently, to limit any potential non-tumor specific reactivity.

The methods described herein may comprise introducing into the subject a therapeutically effective amount of genetically engineered host cells such as hematopoietic cells, for example, immune cells (e.g., αβ T, γδ T or NKT cells), which co-express a factor that redirects glucose metabolites and a TCR polypeptide. The subject (e.g., a human patient such as a human cancer patient) may additionally have been treated or is being treated with an anti-cancer or anti-infection therapy including, but not limited to, an anti-cancer therapeutic agent or anti-infection agent.

The TCR has an antigen-binding domain that may bind any target antigen via processed peptide-MHC complex. Such a target antigen may be any molecule that is associated with a disease or condition, including, but are not limited to, tumor antigens, pathogenic antigens (e.g., bacterial, fungal, or viral), or antigens present on diseased cells, such as those described herein. In some embodiments, the target antigen binding domain targets a native tumor antigen protein. In other embodiments, the target antigen binding domain targets a variant (e.g., mutation) of a tumor antigen protein.

Host cells (e.g., hematopoietic cells, for example, immune cells such as T and NKT cells) expressing factors that redirect glucose metabolites and TCR polypeptides described herein are useful for inhibiting cells expressing a target antigen and/or for enhancing growth and/or proliferation of immune cells in a low-glucose environment, a low amino acid environment, a low pH environment, and/or a hypoxic environment, for example, in a tumor microenvironment. In some embodiments, the subject is a mammal, such as a human, monkey, mouse, rabbit, or domestic mammal. In some embodiments, the subject is a human. In some embodiments, the subject is a human cancer patient. In some embodiments, the subject has additionally been treated or is being treated with any of the therapeutic antibodies described herein.

To practice the method described herein, an effective amount of the hematopoietic cells, for example, immune cells (NKT and/or T cells) expressing any of the factors that redirect glucose metabolites and the TCR polypeptides described herein, or compositions thereof may be administered to a subject in need of the treatment via a suitable route, such as intravenous or subcutaneous administration. As used herein, an effective amount refers to the amount of the respective agent (e.g., the NKT and/or T cells expressing factors that redirect glucose metabolites, TCR polypeptides, or compositions thereof) that upon administration confers a therapeutic effect on the subject. Determination of whether an amount of the cells or compositions described herein achieved the therapeutic effect would be evident to one of skill in the art and has been disclosed herein.

The methods of the disclosure may be used for treatment of any cancer or any pathogen. Specific non-limiting examples of cancers which can be treated by the methods of the disclosure include, for example, lymphoma, breast cancer, gastric cancer, neuroblastoma, osteosarcoma, lung cancer, skin cancer, prostate cancer, colorectal cancer, renal cell carcinoma, ovarian cancer, rhabdomyosarcoma, leukemia, mesothelioma, pancreatic cancer, head and neck cancer, retinoblastoma, glioma, glioblastoma, thyroid cancer, hepatocellular cancer, esophageal cancer, and cervical cancer. In certain embodiments, the cancer may be a solid tumor. The methods of this disclosure may also be used for treating infectious diseases, which may be viral infection.

In some embodiments, the immune cells are administered to a subject in an amount effective in inhibiting cells expressing the target peptide antigen by least 20% and/or by at least 2-fold, e.g., inhibiting cells expressing the target antigen by 50%, 80%, 100%, 2-fold, 5-fold, 10-fold, 20-fold, 50-fold, 100-fold, or more. Additional therapeutic agents (e.g., antibody-based immunotherapeutic agents) may be used to treat, alleviate, or reduce the symptoms of any disease or disorder for which the therapeutic agent is considered useful in a subject.

The efficacy of the cell-based immunotherapy as described herein may be assessed by any method known in the art and would be evident to a skilled medical professional. In some embodiments, the immune cells are administered to a subject in need of the treatment in an amount effective in enhancing the efficacy of an cell-based immunotherapy by at least 20% and/or by at least 2-fold, e.g., enhancing the efficacy of an antibody-based immunotherapy by 50%, 80%, 100%, 2-fold, 5-fold, 10-fold, 20-fold, 50-fold, 100-fold or more, as compared to the efficacy in the absence of the immune cells expressing the factors that redirect glucose metabolites and/or the TCR polypeptide.

In any of the compositions or methods described herein, the immune cells (e.g., NKT and/or T cells) may be autologous to the subject, i.e., the immune cells may be obtained from the subject in need of the treatment, genetically engineered for expression of the factors that redirect glucose metabolites and/or the TCR polypeptides, and then administered to the same subject. In one specific embodiment, prior to re-introduction into the subject, the autologous immune cells (e.g., T or NKT cells) are activated and/or expanded ex vivo. Administration of autologous cells to a subject may result in reduced rejection of the host cells as compared to administration of non-autologous cells. NKT and T cells (αβ T or γδ T cells) can be activated by any method known in the art,

In one embodiment of the described compositions or methods, introduction (or re-introduction) of T cells or NKT cells to the subject is followed by administering to the subject a therapeutically effective amount of IL-2. In additional aspects, the method further comprises cryopreserving the population of engineered iPSCs, NKT or T cells.

In accordance with the present disclosure, patients can be treated by infusing therapeutically effective doses of immune cells such as T or NKT cells comprising a factor that redirects glucose metabolites and/or a TCR polypeptide of the disclosure in the range of about 10⁵ to 10¹⁰ or more cells per kilogram of body weight (cells/Kg). The infusion can be repeated as often and as many times as the patient can tolerate until the desired response is achieved. The appropriate infusion dose and schedule will vary from patient to patient, but can be determined by the treating physician for a particular patient. Typically, initial doses of approximately 10⁶ cells/kg will be infused, escalating to 10⁸ or more cells/kg.

The efficacy of the compositions or methods described herein may be assessed by any method known in the art and would be evident to a skilled medical professional. For example, the efficacy of the compositions or methods described herein may be assessed by survival of the subject or cancer or pathogen burden in the subject or tissue or sample thereof. In some embodiments, the compositions and methods described herein may be assessed based on the safety or toxicity of the therapy (e.g., administration of the immune cells expressing the factors that redirect glucose metabolites and the TCR polypeptides) in the subject, for example, by the overall health of the subject and/or the presence of adverse events or severe adverse events.

### V. Combination Treatments

The compositions and methods described in the present disclosure may be utilized in conjunction with other types of therapy for cancer, such as chemotherapy, surgery, radiation, gene therapy, and so forth, or anti-infection therapy. Such therapies can be administered simultaneously or sequentially (in any order) with the immunotherapy according to the present disclosure. When co-administered with an additional therapeutic agent, suitable therapeutically effective dosages for each agent may be lowered due to the additive action or synergy.

In some embodiments, the immune cells (e.g., T and/or NK cells) expressing any of the factors that redirect glucose metabolites and/or the chimeric receptor polypeptides disclosed herein may be administered to a subject who has been treated or is being treated with an additional therapeutic agent (e.g., an additional anti-cancer therapeutic agent). For example, the immune cells may be administered to a human subject simultaneously with the additional therapeutic agent. In some embodiments, the immune cells may be administered to a human subject before the additional therapeutic agent. In some embodiments, the immune cells may be administered to a human subject after the additional therapeutic agent.

Genetically engineered immune cells (e.g., T cells or NK cells) that co-express a factor that redirects glucose metabolites and a CAR polypeptide specific to a tag can be co-used with a therapeutic agent conjugated to the tag. Via the therapeutic agent, which is capable of binding to an antigen associated with diseased cells such as tumor cells, such genetically engineered immune cells can be engaged with the diseased cells and inhibit their growth. Any of the antibodies listed in Table 4 above, or others specific to the same target antigen also listed in Table 4 can be conjugated to a suitable tag (e.g., those described herein) and be co-used with immune cells co-expressing the factor that redirects glucose metabolites and a CAR polypeptide specific to the tag.

The treatments of the disclosure can be combined with other immunomodulatory treatments such as, e.g., therapeutic vaccines (including but not limited to GVAX, dendritic cell (DC)-based vaccines, etc.), checkpoint inhibitors (including but not limited to agents that block CTLA4, PD1, LAG3, TIM3, etc.) or activators (including but not limited to agents that enhance 41BB, OX40, etc.). In some embodiments, genetically engineered immune cells (e.g., T cells or NK cells) that co-express a factor that redirects glucose metabolites, and a CAR polypeptide is combined with an immunomodulatory treatment.

Non-limiting examples of other therapeutic agents useful for combination with the immunotherapy of the disclosure include: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by (Carmeliet and Jain, Nature, 407(6801): 249-257 (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones, and navelbine, epidipodophyllotoxins (etoposide and teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamine oxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycin, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (brefeldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker, nitric oxide donors; anti-sense oligonucleotides; antibodies (trastuzumab); cell cycle inhibitors and differentiation inducers (tretinoin); AKT inhibitors (such as MK-2206 2HCl, Perifosine (KRX-0401), GSK690693, Ipatasertib (GDC-0068), AZD5363, uprosertib, afuresertib, or triciribine); mTOR inhibitors, topoisomerase inhibitors (doxorubicin (adriamycin), amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

For examples of additional useful agents see also Physician's Desk Reference, 59^{th} edition, (2005), Thomson P D R, Montvale N.J.; Gennaro et al., Eds. Remington's The Science and Practice of Pharmacy 20^{th} edition, (2000), Lippincott Williams and Wilkins, Baltimore Md.; Braunwald et al., Eds. Harrison's Principles of Internal Medicine, 15^{th} edition, (2001), McGraw Hill, NY; Berkow et al., Eds. The Merck Manual of Diagnosis and Therapy, (1992), Merck Research Laboratories, Rahway N.J.

The administration of an additional therapeutic agent can be performed by any suitable route, including systemic administration as well as administration directly to the site of the disease (e.g., to a tumor).

In some embodiments, the method involves administering the additional therapeutic agent (e.g., an antibody) to the subject in one dose. In some embodiments, the method involves administering the additional therapeutic agent (e.g., an antibody) to the subject in multiple doses (e.g., at least 2, 3, 4, 5, 6, 7, or 8 doses). In some embodiments, the additional therapeutic agent (e.g., an antibody) is administered to the subject in multiple doses, with the first dose of the additional therapeutic agent (e.g., an antibody) administered to the subject about 1, 2, 3, 4, 5, 6, or 7 days prior to administration of the immune cells expressing the factor that redirects glucose metabolites and/or the CAR polypeptide. In some embodiments, the first dose of the additional therapeutic agent (e.g., an antibody) is administered to the subject between about 24-48 hours prior to the administration of the immune cells expressing the factor that redirects glucose metabolites and/or the CAR polypeptide. In some instances, the additional therapeutic agent can be an antibody specific to a target antigen of interest, for example, those listed in Table 4 and others that are specific to the same target.

In some embodiments, the first dose of the additional therapeutic agent (e.g., an antibody) is administered to the subject prior to the administration of the immune cells expressing the factor that redirects glucose metabolites and/or the CAR polypeptide. In some embodiments, the additional therapeutic agent (e.g., an antibody) is administered to the subject prior to administration of the immune cells expressing the factor that redirects glucose metabolites and/or the CAR polypeptide and then subsequently about every two weeks. In some embodiments, the first two doses of the additional therapeutic agent (e.g., an antibody) are administered about one week (e.g., about 6, 7, 8, or 9 days) apart. In certain embodiments, the third and following doses are administered about every two weeks.

In any of the embodiments described herein, the timing of the administration of the additional therapeutic agent (e.g., an antibody) is approximate and includes three days prior to and three days following the indicated day (e.g., administration every three weeks encompasses administration on day 18, day 19, day 20, day 21, day 22, day 23, or day 24).

Efficacy of immune system induction for disease therapy may be enhanced by combination with other agents that, for example, that reduces tumor burden prior to administration of CAR-T or CAR-NK. Antibody-drug conjugates (ADCs) can efficiently reduce tumor burden in many types of cancers. Numerous exemplary ADCs are known in the art (Mullard, Nat Rev Drug Discov, 12(5): 329-332 (2013); Coats et al., Clinical Cancer Research, 25(18): 5441-5448 (2019); Zhao et al., Acta Pharmaceutica Sinica B, 10(9): 1589-1600 (2020); Fu et al., Signal Transduction and Targeted Therapy, 7(1): 93 (2022)). Any such known ADC may be used in combination with a CAR-T or CAR-NK construct as described herein. Thus, in some embodiments, where an ADC is used in combination with a CAR-T or CAR-NK, the ADC is administered prior to the CAR-T or CAR-NK. In some embodiments, an ADC is used in combination with a CAR-T or CAR-NK as disclosed herein. In some embodiments, the first dose of the ADC is administered to the subject prior to the administration of the immune cells expressing the factor that redirects glucose metabolites and/or the CAR polypeptide.

In another embodiment, the efficacy of the immune system induction for the disease therapy may be enhanced by combination with other immunotherapeutic agents, e.g., cytokines that stimulate the CAR-T or CAR-NK cells in vivo (e.g., agonists of the IL-2/IL-15Rβγ such as IL-2, IL-15 (IL-2/IL-15 superagonists); IL-7, or IL-12, or derivatives thereof) or immune checkpoint inhibitors (e.g., anti-PD-1 antibodies, anti-PD-L1 antibodies, anti-LAG3 antibodies, anti-CTLA4 antibodies or anti-TIM3 antibodies).

In some embodiments, the method further comprises administering a lymphocyte reduction treatment, preferably selected from cyclophosphamide and fludarabine. Such lymphodepletion treatment is preferably applied prior to the infusion of the hematopoietic cells expressing a CAR in order to allow for greater T cell expansion of the infused cells (Shank et al., Pharmacotherapy, 37(3): 334-345 (2017)).

The efficacy of the methods described herein may be assessed by any method known in the art and would be evident to a skilled medical professional and/or those described herein. For example, the efficacy of the antibody-based immunotherapy may be assessed by survival of the subject or cancer burden in the subject or tissue or sample thereof. In some embodiments, the antibody-based immunotherapy is assessed based on the safety or toxicity of the therapy in the subject, for example by the overall health of the subject and/or the presence of adverse events or severe adverse events.

### VI. Kits for Therapeutic Use

The present disclosure also provides kits for use of the compositions described herein. For example, the present disclosure also provides kits comprising a population of immune cells (e.g., T or NK cells, constructed in vitro or in vivo) that express a factor that redirects glucose metabolites and optionally a chimeric receptor polypeptide for use in inhibiting the growth of diseased cells, e.g., tumor cells and/or enhancing immune cell growth and/or proliferation in a low glucose environment, a low amino acid environment, a low-pH environment, and/or hypoxic environment, for example, in a tumor microenvironment. The kit may further comprise a therapeutic agent or a therapeutic agent conjugated to a tag (e.g., those described herein), to which the chimeric receptor polypeptide expressed on the immune cells bind. Such kits may include one or more containers comprising the population of the genetically engineered immune cells as described herein (e.g., T and/or NK cells), which co-express a factor that redirects glucose metabolites and a chimeric receptor polypeptide such as those described herein, and optionally a therapeutic agent or a therapeutic agent conjugated to a tag.

In some embodiments, the kit comprises factor that redirects glucose metabolites-expressing and chimeric receptor polypeptide-expressing immune cells, which are expanded ex vivo. In another embodiment, the kit comprises factor that redirects glucose metabolites-expressing and chimeric receptor polypeptide-expressing immune cells and an antibody specific to a cell surface antibody that is present on activated T cells, for example, an anti-CD5 antibody, an anti-CD38 antibody or an anti-CD7 antibody. In exemplary embodiments, the kit comprises factor that redirects glucose metabolites-expressing and CAR-expressing NK or T cells. The factor that redirects glucose metabolites-expressing and chimeric receptor polypeptide-expressing immune cells may express any of the chimeric receptor polypeptide constructs known in the art or disclosed herein.

Alternatively, the kit disclosed herein may comprise a nucleic acid or a nucleic acid set as described herein, which collectively encodes any of the chimeric receptor polypeptides and any of the factors that redirect glucose metabolites as also described herein.

In some embodiments, the kit can additionally comprise instructions for use in any of the methods described herein. The included instructions may comprise a description of administration of the first and second pharmaceutical compositions to a subject to achieve the intended activity, e.g., inhibiting target cell growth in a subject, and/or enhancing the growth and/or proliferation of immune cells in a low-glucose environment, a low amino acid (e.g., a low glutamine environment) environment, a low pH environment, and/or a hypoxic environment (e.g., a low glucose, low amino acid, low pH or hypoxic tumor microenvironment). The kit may further comprise a description of selecting a subject suitable for treatment based on identifying whether the subject is in need of the treatment. In some embodiments, the kit may further comprise a description of selecting a subject suitable for treatment based on identifying whether the subject is in need of the treatment. Non-limiting examples of such methods of identification may include expression of target in blood, DNA or tissue (e.g., immunohistochemistry). Further, in some instances, a cut-off range may be used to adjust treatment dosage.

In some embodiments, the instructions comprise a description of administering the population of genetically engineered immune cells (e.g., T or NK cells) and optionally a description of administering the tag-conjugated therapeutic agent. The instructions relating to the use of the immune cells (e.g., T or NK cells) and optionally the tag-conjugated therapeutic agent as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the second pharmaceutical composition is an antibody as described herein. At least one active agent in the first pharmaceutical composition is a population of immune cells (e.g., T lymphocytes or NK cells) that express a chimeric receptor polypeptide and a factor that redirects glucose metabolites as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

A further embodiment of the present invention is a nucleic acid or nucleic acid set, which collectively comprises: a first nucleotide sequence encoding the factor that diverts or redirects glucose metabolites set forth above; and a second nucleotide sequence encoding the chimeric receptor polypeptide set forth above. The isolated nucleic acid or nucleic acid set may be used to manufacture the genetically engineered immune cell of the invention. Accordingly, a further embodiment is a method of manufacturing modified immune cells in vitro by transfecting immune cells with nucleic acid or nucleic acid sets, and selecting/enriching transfected immune cells. Selection for transfected cells may be performed by selecting for the expression of the chimeric receptor polypeptide on the surface of the immune cells by methods know in the art (e.g., Flow cytometry). The immune cells optionally can be activated in vitro by any method known in the art as described above for NK and T cells.

Such isolated nucleic acids or nucleic acid sets may be in the form of a vector or a set of vectors. Accordingly, a method for generating modified immune cells in vivo, the method comprising administering to a subject in need thereof the nucleic acid or nucleic acid set according to the invention, preferably a vector or set of vectors. Respective immune cells of the subject would be transfected with the nucleic acids or nucleic acid sets (e.g., a gamma-retrovirus or lentivirus) and leading to expression or overexpression of the polypeptide that diverts or redirects glucose metabolites out of a glycolysis pathway, and the chimeric receptor polypeptide

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985); Transcription and Translation (B. D. Hames & S. J. Higgins, eds. (1984»; Animal Cell Culture (R. I. Freshney, ed. (1986); Immobilized Cells and Enzymes (IRL Press, (1986); and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are intended only to illustrate methods and embodiments in accordance with the invention, and as such should not be construed as imposing limitations upon the claims.

### Example 1: Impact of Expressing a Factor that Redirects Glucose Metabolites Out of the Glycolysis Pathway on Immune Cell Function Expressing an ACTR Polypeptide in Lower Glucose Environments

A transgene encoding for a factor (e.g., a polypeptide) that redirects glucose metabolites is co-expressed in the same T and/or NK cell with an ACTR polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells are transduced with a virus encoding the ACTR polypeptide and the factor that redirects glucose metabolites separated, for example, by a P2A ribosomal skip sequence. The T and/or NK cells are mixed at a given effector-to-target (E:T) ratio with tumor target cells, such as IGROV-1 cells, and a tumor-targeting antibody such as an anti-FOLRα antibody. Reactions are then incubated at 3TC in a 5% CO₂ incubator for a period of time (e.g., 6-8 days) at different starting concentrations of glucose (e.g., 0-20 mM). T and/or NK cell function is then evaluated, for example, using cytokine production or proliferation assays or for resistance to chronic stimulation. Cytokine production (e.g., IL-2 and/or IFN-γ) is measured from the reaction supernatant. For proliferation experiments, co-cultures are harvested and stained with α-CD3, α-CD14, α-CD33, α-CD45, α-CD56 antibodies and a live-dead cell stain. As a measure of T cell proliferation, the live T cells is enumerated in CD45⁺CD33⁻CD3⁺CD14⁻CD56⁻ cells and a live-dead cell stain is evaluated by flow cytometry. And in case of NK cells, enumeration is carried on CD45⁺CD33⁻CD3⁻CD14⁻CD56⁺ and a live-dead cell stain is evaluated by flow cytometry.

T and/or NK cells expressing a factor that redirects glucose metabolites in addition to the ACTR polypeptide show enhanced T and/or NK cell function relative to T cells expressing ACTR alone including, for example, enhanced cytokine production or enhanced proliferation. This enhanced function may be more pronounced at lower glucose concentrations. These experiments demonstrate that expressing a factor that redirects glucose metabolites in immune (such as T and/or NK) has a positive impact on immune cell activity.

### Example 2: Impact of Expressing a Factor that Redirects Glucose Metabolites Out of the Glycolysis Pathway on Immune Cell Function Expressing an ACTR Polypeptide in Environments with Higher Soluble Inhibitor Concentrations

A transgene encoding for a factor (e.g., a polypeptide) that redirects glucose metabolites is co-expressed in the same T and/or NK cell with an ACTR polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells are transduced with a virus encoding the ACTR polypeptide and the factor that redirects glucose metabolites separated, for example, by a P2A ribosomal skip sequence. The T and/or NK cells are mixed at a given effector-to-target (E:T) ratio with tumor target cells, such as IGROV-1 cells, and a tumor-targeting antibody such as an anti-FOLRα antibody, in media containing different concentrations of soluble inhibitors that are present in the tumor microenvironment (e.g., TGFβ, PGE₂, kynurenine, and/or adenosine). Reactions are then incubated at 37° C. in a 5% CO₂ incubator for a period of time (e.g., 6-8 days). NK and/or T cell function is then evaluated, for example, using cytokine production or proliferation assays or for resistance to chronic stimulation. Cytokine production (e.g., IL-2 and/or IFN-γ) is measured from the reaction supernatant. For proliferation experiments, co-cultures of NK and/or T cells are harvested, stained and evaluated by flow cytometry (see Example 1).

T and/or NK cells expressing a factor that redirects glucose metabolites in addition to the polypeptide show enhanced cellular function relative to NK and/or T cells expressing ACTR alone including, for example, enhanced cytokine production. This enhanced function may be achieved at higher soluble inhibitor concentrations. These experiments demonstrate that expressing a factor that redirects glucose metabolites in immune (such as T or NK) cells has a positive impact on immune cell activity.

### Example 3: Impact of Expressing a Factor that Redirects Glucose Metabolites Out of the Glycolysis Pathway on Immune Cell Function Expressing an ACTR Polypeptide in Environments with Greater Immunosuppressive Cell Presence

A transgene encoding for a factor (e.g., a polypeptide) that redirects glucose metabolites is co-expressed in the same T and/or NK cell with an ACTR polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells are transduced with a virus encoding the ACTR polypeptide and the factor that redirects glucose metabolites separated, for example, by a P2A ribosomal skip sequence. The T and/or NK cells are mixed at a given effector-to-target (E:T) ratio with tumor target cells, such as IGROV-1 cells, and a tumor-targeting antibody such as an anti-FOLRα, antibody, in the presence of immunosuppressive cells (e.g., myeloid-derived suppressor cells and/or regulatory T cells). Reactions are then incubated at 37° C. in a 5% CO₂ incubator for a period of time (e.g., 3-10 days). Immune cell (NK and/or T cell) function is then evaluated, for example, using cytokine production or cell proliferation assays or for resistance to chronic stimulation. Cytokine production (e.g., IL-2 and/or IFN-γ) is measured from the reaction supernatant. Proliferation experiments is performed and evaluated as described in example 1.

T and/or NK cells expressing a factor that redirects glucose metabolites in addition to the ACTR or CAR polypeptide show enhanced T and/or NK cell function relative to T and/or NK cells expressing ACTR or CAR alone including, for example, enhanced cytokine production or enhanced proliferation. This enhanced function may be achieved in the presence of increased amounts (e.g., greater number or percentage) of immunosuppressive cells. These experiments demonstrate that expressing a factor that redirects glucose metabolites in immune (such as T and/or NK) cells has a positive impact on immune cell activity.

### Example 4: Impact of Expressing a Factor that Redirects Glucose Metabolites Out of the Glycolysis Pathway on Immune Cell Function Expressing an ACTR Polypeptide on Tumor Models

A transgene encoding for a factor (e.g., a polypeptide) that redirects glucose metabolites is co-expressed in the same T and/or NK cell with an ACTR polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells are transduced with a virus encoding the ACTR polypeptide and the factor that redirects glucose metabolites separated, for example, by a P2A ribosomal skip sequence. Transduced T and/or NK cells are evaluated for anti-tumor activity in mouse tumor models. For these experiments, a tumor cell line, for example IGROV-1, is inoculated into NSG™ (NOD scid gamma, NOD.Cg-Prkdc^{scid} IL2rg^{tm1Wkl}/SzJ, Strain 005557) mice. Tumor-bearing mice are subsequently dosed with a tumor-targeting antibody, for example an anti-FOLRα antibody, and T and/or NK cells expressing ACTR alone or ACTR and a factor that redirects glucose metabolites. Tumor growth is monitored throughout the course of the experiment.

In combination with a tumor-targeting antibody, T and/or NK cells expressing a factor that redirects glucose metabolites in addition to an ACTR polypeptide show enhanced anti-tumor activity relative to T and/or NK cells expressing an ACTR polypeptide alone. Additionally, in combination with a tumor-targeting antibody, T and/or NK cells expressing a factor that redirects glucose metabolites in addition to an ACTR polypeptide may show enhanced T and/or NK cell activity including, for example, enhanced proliferation, enhanced T and/or NK cell persistence, and/or enhanced cytokine production relative to T and/or NK cells expressing the ACTR polypeptide alone. These experiments demonstrate that expressing a factor that redirects glucose metabolites in ACTR-expressing immune (such as T and/or NK) cells has a positive impact on immune cell function in vivo.

### Example 5: Impact of Reduced Glucose Concentrations on T Cell Function

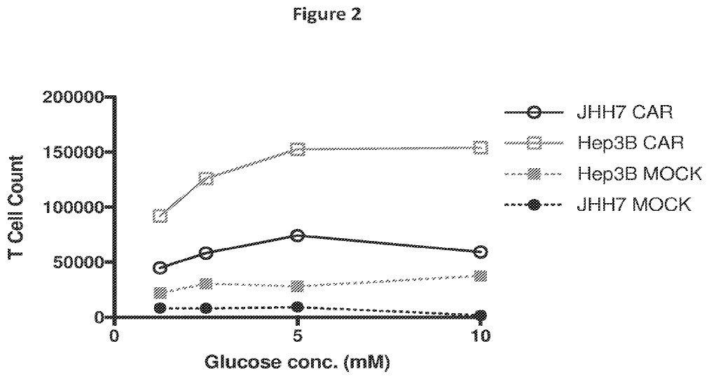

Gamma-retrovirus encoding an exemplary GPC3-targeting CAR expression construct of SEQ ID NO: 78 was generated via recombinant technology and used to infect primary human T-cells for generating cells that express a GPC3-targeting CAR polypeptide on their cell surface. A six-day flow-based proliferation assay was then used to test the functionality of the GPC3-targeting CAR expressing cells. Specifically, 200,000 untransduced mock T-cells or T-cells expressing the GPC3-targeting CAR construct were incubated together at a ratio of 4:1 (effector cells/CAR-expressing T cells to target cells) with either 50,000 GPC3⁺ hepatocellular carcinoma JHH7 or Hep3B tumor cells. The co-culture was incubated at 37° C. in a 5% CO₂ incubator for six days in the presence of different concentrations of glucose. At the end of six days, co-cultures were harvested and stained with an anti-CD3 antibody. The number of CD3-positive cells was evaluated by flow cytometry as a measure of T cell proliferation. At lower glucose concentrations, less CAR-T proliferation was observed (FIG. 2). These experiments demonstrate that low glucose environments may have a negative impact on CAR-T cell proliferation activity.

### Example 6: Impact of Expressing a Factor that Redirects Glucose Metabolites Out of the Glycolysis Pathway on Immune Cell Function Using a GPC3-Targeting CAR-T or CAR-NK Expression Construct

Gamma-retrovirus encoding an exemplary GPC3-targeting CAR polypeptide expression construct (SEQ ID NO: 78 or SEQ ID NO: 79) is generated via recombinant technology and used to infect primary human T and/or or NK cells to generate cells expressing a GPC3-targeting CAR polypeptide on their cell surface. Additionally, gamma-retroviruses encoding an exemplary GPC3-targeting CAR polypeptide and a factor that redirects glucose metabolites out of the glycolysis pathway (PKM2, PKM2 Y105E variant, PKM2 Y105D variant, PKM2 K422R variant, PKM2 H391Y variant, GFPT1, or TIGAR) (SEQ ID NO: 68-SEQ ID NO: 74) are generated via recombinant technology and used to infect primary human T and/or NK cells to generate cells that express a GPC3-targeting polypeptide and a factor that redirects glucose metabolites. In the constructs encoding both the CAR polypeptide and the factor that redirects glucose metabolites, the two polypeptides are separated, for example, by a P2A ribosomal skip sequence. The variants to be expressed are SEQ ID NO: 68-SEQ ID NO: 74 (for, e.g., CAR+PKM2, CAR+PKM2 Y105E, CAR+PKM2 Y105D, CAR+PKM2 K422R, CAR+PKM2 H391Y, CAR+GFPT1, or CAR+TIGAR). A six-day flow-based proliferation assay is then used to test the functionality of the GPC3-targeting CAR expressing cells. Specifically, 200,000 untransduced mock T or NK cells, T or NK cells expressing a GPC3-targeting CAR polypeptide, or T or NK cells expressing a GPC3-targeting CAR polypeptide and a factor that redirects glucose metabolites are incubated together at a ratio of 4:1 (effector cells/CAR-expressing T or NK cells to target cells) with 50,000 GPC3⁺ hepatocellular carcinoma JHH7 tumor cells. The co-culture is incubated at 37° C. in a 5% C02 for six days in the presence of 1.25 mM glucose (tumor-relevant) and 10 mM glucose (approximate peripheral blood levels). At the end of six days, co-cultures are harvested, and co-cultures are harvested and stained with α-CD3, α-CD14, α-CD33, α-CD45, α-CD56 antibodies and a live-dead cell stain. As a measure of T cell proliferation, the live T cells is enumerated in CD45⁺CD33⁻CD3⁺CD14⁻CD56⁻ cells and a live-dead cell stain is evaluated by flow cytometry. And in case of NK cells, enumeration is carried on CD45⁺CD33⁻CD3⁻CD14⁻CD56⁺ and a live-dead cell stain is evaluated by flow cytometry.

Immune cells expressing the factor that redirects glucose metabolites in addition to the CAR polypeptide demonstrate enhanced T and/or NK cell proliferation relative to T and/or NK cells expressing the CAR construct alone. This enhanced proliferation also occurs at tumor-relevant low glucose concentrations. These experiments demonstrate that expressing a factor that redirects glucose metabolites in immune (such as T and/or NK) cells has a positive impact on CAR-T and/or CAR-NK cell proliferation activity.

### Example 7: Impact of Expressing a Factor that Redirects Glucose Metabolites Out of the Glycolysis Pathway on Immune Cell Function Expressing a CAR Polypeptide in Environments with Higher Soluble Inhibitor Concentrations

A transgene encoding for a factor (e.g., a polypeptide) that redirects glucose metabolites is co-expressed in the same T and/or NK cell with a CAR polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells are transduced with virus encoding the CAR polypeptide and the factor that redirects glucose metabolites separated, for example, by a P2A ribosomal skip sequence. Transduced T and/or NK cells are mixed at a given effector-to-target (E:T) ratio with tumor target cells, such as HepG2 cells, in media containing different concentrations of soluble inhibitors that are present in the tumor microenvironment (e.g., TGFβ, PGE₂, kynurenine, and/or adenosine). Reactions are then incubated at 37° C. in a 5% CO₂ incubator for a period of time (e.g., 6-8 days). NK and/or T cell function is then evaluated, for example, using cytokine production or proliferation assays or for resistance to chronic stimulation. Cytokine production (e.g., IL-2 and/or IFN-γ) is measured from the reaction supernatant. For proliferation experiments, co-cultures of NK and/or T cells are harvested, stained and evaluated by flow cytometry (see Example 1).

T and/or NK cells expressing a factor that redirects glucose metabolites in addition to the CAR polypeptide show enhanced T and/or NK cell function relative to T and/or NK cells expressing CAR alone including, for example, enhanced cytokine production or enhanced proliferation. This enhanced function may be achieved at higher soluble inhibitor concentrations. These experiments demonstrate that expressing a factor that redirects glucose metabolites in immune (such as T and/or NK) cells has a positive impact on immune cell activity.

### Example 8: Impact of Expressing a Factor that Redirects Glucose Metabolites Out of the Glycolysis Pathway on Immune Cell Function Expressing a CAR Polypeptide in Environments with Greater Immunosuppressive Cell Presence

A transgene that encodes a factor that redirects glucose metabolites is co-expressed in the same T and/or NK cell with a CAR polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells are transduced with virus encoding the CAR polypeptide and the factor that redirects glucose metabolites separated, for example, by a P2A ribosomal skip sequence. Transduced T and/or NK cells are mixed at a given effector-to-target (E:T) ratio with tumor target cells, such as HepG2 cells, in the presence of immunosuppressive cells (e.g, myeloid-derived suppressor cells and/or regulatory T cells). Reactions are then incubated at 37° C. in a 5% CO₂ incubator for a period of time (e.g., 3-10 days). T and/or NK cell function is then evaluated, for example, using cytokine production or cell proliferation assays or for resistance to chronic stimulation. Cytokine production (e.g., IL-2 and/or IFN-γ) is measured from the reaction supernatant. Proliferation experiments is performed and evaluated as described in example 1.

T and/or NK cells expressing a factor that redirects glucose metabolites in addition to the CAR polypeptide show enhanced T and/or NK cell function relative to T and/or NK cells expressing CAR alone including, for example, enhanced cytokine production or enhanced proliferation. This enhanced function may be achieved in the presence of increased amounts (e.g., greater number or percentage) of immunosuppressive cells. These experiments demonstrate that expressing a factor that redirects glucose metabolites in immune (such as T and/or NK) cells has a positive impact on immune cell activity.

### Example 9: Impact of Expressing a Factor that Redirects Glucose Metabolites Out of the Glycolysis Pathway on T Cell Function in Tumor Models

A transgene that encodes a factor that redirects glucose metabolites is co-expressed in the same T and/or NK cell with a chimeric antigen receptor (CAR) polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells are transduced with virus encoding the CAR polypeptide and the factor that redirects glucose metabolites separated, for example, by a P2A ribosomal skip sequence. Transduced T and/or NK cells are evaluated for anti-tumor activity in mouse tumor models. For these experiments, a tumor cell line, for example HepG2, is inoculated into NSG™ (NOD scid gamma, NOD.Cg-Prkdc^{scid} IL2rg^{tmWjl}/SzJ, Strain 005557) mice. Tumor-bearing mice are subsequently dosed with T and/or NK cells expressing CAR alone or CAR and a factor that redirects glucose metabolites. Tumor growth is monitored throughout the course of the experiment.

T and/or NK cells expressing a factor that redirects glucose metabolites in addition to a CAR polypeptide show enhanced anti-tumor activity relative to T or NK cells expressing a CAR polypeptide alone. Additionally, T and/or NK cells expressing a factor that redirects glucose metabolites in addition to a CAR polypeptide may show enhanced T and/or NK cell activity including, for example, enhanced proliferation, persistence, and/or cytokine production relative to T and/or NK cells expressing the CAR polypeptide alone. These experiments demonstrate that expressing a factor that redirects glucose metabolites in CAR-expressing T and/or NK cells has a positive impact on T or NK cell function in vivo.

### Example 10: Expression of GLUT1, GOT2, and TIGAR Elevated Glucose Uptake and Lactate Production

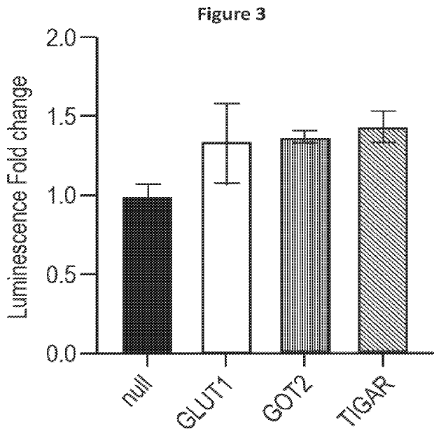

Healthy donor PBMCs were stimulated with anti-CD3 and anti-CD28 until day 2 followed by transduction with V5-tagged transgene packaged into a lentiviral vector. The transgene encodes GLUT1 (SEQ ID NO: 80), GOT2 (SEQ ID NO: 81), or TIGAR (SEQ ID NO: 69). The transduced cells were supplemented with fresh IL-2 each day until day 10. 10,000 cells/well (384-well plate) were resuspended in PBS and assayed for glucose uptake. The luminescence read-out was evaluated as a fold change for each transgene were compared to null (non-transduced control; baseline as fold change 1) T cells under the same condition. Cells transduced with GLUT1, GOT2 or TIGAR showed elevated levels of glucose uptake, which is indicative of higher metabolic activity. Data are representative of three donors. See FIG. 3.

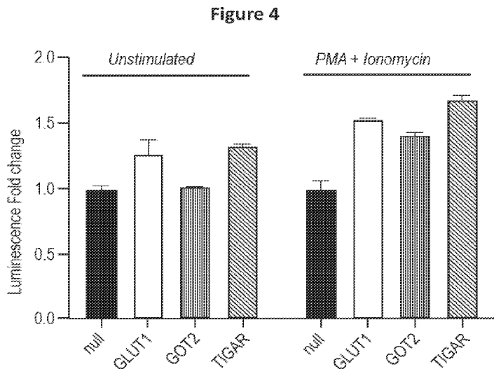

Further, healthy donor PBMCs were stimulated with anti-CD3 and anti-CD28 until day 2 followed by transduction with V5-tagged transgene (described above) packaged into lentiviral vectors. The transduced cells were supplemented with fresh IL-2 each day until day 9. On day 9, a subset of T cells was stimulated with PMA and Ionomycin for 24 h. 10,000 harvested cells/well (384-well plate) were resuspended in RPMI without FBS and incubated at 37° C. for 2 h to remove residual lactate from the media and assayed for lactate production. The luminescence read-out was evaluated as a fold change for each transgene were compared to null (non-transduced control; baseline as fold change 1) T cells under the same condition. Stimulated cells transduced with GLUT1, GOT2 and TIGAR showed elevated levels of lactate production indicative of higher metabolic adaptability in nutrient deficient environments. See FIG. 4.

In sum, the results of this example show that T cells transduced with GLUT1, GOT2, or TIGAR showed enhanced metabolic activity and adaptability in nutrient deficient environment and TIGAR showed the best effects among the three. This indicates that therapeutic T cells (e.g., T cells expressing an ACTR or CAR polypeptide as disclosed herein) co-expressing GLUT1, GOT2, or TIGAR (specifically TIGAR) would be better adapted to tumor microenvironment (which could be deficient in nutrient) and exhibit better therapeutic activity as compared with counterpart T cells that are not transduced with the GLUT1, GOT2, or TIGAR gene. See also WO2020/010110 and WO2020/037066, the relevant disclosures of each of which are incorporated by reference for the subject matter and purpose referenced herein.

### Example 11: Impact of Expressing a Factor that Redirects Glucose Metabolism in Immune Cells Expressing an ACTR Polypeptide

A transgene encoding a factor (e.g., a polypeptide) that redirects glucose metabolites is co-expressed in the same T and/or NK cell expressing an ACTR polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells are stimulated with anti-CD3 and anti-CD28 for a time period (e.g., 1-4 days) followed by transduction with virus (e.g., lentivirus or gamma-retrovirus) encoding the ACTR polypeptide and the factor that redirects glucose metabolites, which can be separated, for example, by a P2A ribosomal skip sequence. The transduced cells are supplemented with cytokines (e.g., IL-2) for 3-10 days. All reactions are incubated at 37° C. in a 5% CO₂ incubator. For glucose uptake measurements, cells are harvested and assayed for glucose uptake using Glucose Uptake Glo Kit. This luminescence-based assay is evaluated and data represented as a fold change. Complimentary cell metabolic flux assays are performed to capture changes in basal oxygen consumption rate (OCR) using seahorse extracellular flux analyzer.

T and/or NK cells expressing a factor that redirects glucose metabolites in addition to the ACTR polypeptide are expected to show enhanced glucose uptake. This enhanced function is suggestive of increased metabolic fitness and has a positive impact on the immune cell activity.

### Example 12: Impact of Expressing a Factor that Redirects Glucose Metabolism in Immune Cells Expressing a CAR Polypeptide

A transgene encoding a factor (e.g., a polypeptide) that redirects glucose metabolites is co-expressed in the same T and/or NK cell expressing a CAR polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells are stimulated with anti-CD3 and anti-CD28 for a time period (e.g., 1-4 days) followed by transduction with virus (e.g., lentivirus or gamma-retrovirus) encoding the CAR polypeptide and the factor that redirects glucose metabolites, which can be separated, for example, by a P2A ribosomal skip sequence. The transduced cells are supplemented with cytokines (e.g., IL-2) for 3-10 days. All reactions are incubated at 37° C. in a 5% CO₂ incubator. For glucose uptake measurements, cells are harvested and assayed for glucose uptake using Glucose Uptake Glo Kit. This luminescence-based assay is evaluated and data represented as a fold change. Complimentary cell metabolic flux assays are performed to capture changes in basal oxygen consumption rate (OCR) using seahorse extracellular flux analyzer.

T and/or NK cells expressing a factor that redirects glucose metabolites in addition to the CAR polypeptide are expected to show enhanced glucose uptake. This enhanced function is suggestive of increased metabolic fitness and has a positive impact on immune cell activity.

### Example 13: Impact of Expressing a Factor that Redirects Lactate Production in Immune Cells Expressing an ACTR Polypeptide

A transgene encoding for a factor (e.g., a polypeptide) that redirects lactate production is co-expressed in the same T and/or NK cell with an ACTR polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells are stimulated with anti-CD3 and anti-CD28 for a time period (e.g., 1-4 days) followed by transduction with virus (e.g., lentiviral or gamma-retroviral) encoding the ACTR polypeptide and the factor that redirects glucose metabolites separated, for example, by a P2A ribosomal skip sequence. The transduced cells are supplemented with cytokines (e.g., IL-2) and additionally with stimulants (e.g., PMA and/or Ionomycin) for 3-10 days. All reactions are incubated at 37° C. in a 5% CO₂ incubator. Cells are harvested and assayed for lactate production using Lactate Glow Assay. This luminescence-based assay was evaluated, and data represented as a fold change.

T and/or NK cells expressing a factor that redirects lactate production in addition to the ACTR polypeptide are expected to show enhanced lactate production. This enhanced function is suggestive of increased metabolic fitness and has a positive impact on immune cell activity.

### Example 14: Impact of Expressing a Factor that Redirects Lactate Production in Immune Cells Expressing a CAR Polypeptide

A transgene encoding for a factor (e.g., a polypeptide) that redirects lactate production is co-expressed in the same T and/or NK cell with a CAR polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells are stimulated with anti-CD3 and anti-CD28 for a time period (e.g., 1-4 days) followed by transduction with virus (e.g., lentiviral or gamma-retroviral) encoding the CAR polypeptide and the factor that redirects glucose metabolites separated, for example, by a P2A ribosomal skip sequence. The transduced cells are supplemented with cytokines (e.g., IL-2) and additionally with stimulants (e.g., PMA and/or Ionomycin) for 3-10 days. All reactions are incubated at 37° C. in a 5% CO₂ incubator. Cells are harvested and assayed for lactate production using Lactate Glow Assay. This luminescence-based assay was evaluated, and data represented as a fold change.

T and/or NK cells expressing a factor that redirects lactate production in addition to the CAR polypeptide are expected to show enhanced lactate production. This enhanced function is suggestive of increased metabolic fitness and has a positive impact on immune cell activity.

### Example 15: Production of Retroviral Particles

On day 1, 12×10⁶ low passage HEK293T cells were plated on 15 cm coated tissue culture plates in DMEM media containing 10% FBS. The following day, i.e., day 2, the cells were 80% confluent. On day 3, the cells were subjected to transfection. 3 ml of transfection mix containing 10 μg of GAG/Pol, 6.6 μg of GALV helper, 20 μg of transfer plasmids and 74 μl of PEIPro transfection reagent (Cat #115-010, PolyPlus) was prepared and added to the cell culture plates. The transfected cells were replenished with fresh DMEM media containing 10% FBS media 6 h post-transfection. The viral supernatants were harvested at 24 h and 36 h post-transfection and concentrated through a 0.45 μm filter and stored at −80° C. until further use.

### Example 16: Initiation and Transduction of Immune Cells

Immune cells (such as NK and/or T cells) were isolated either from fresh blood samples or are derived from cell lines.

Peripheral blood mononuclear cells (PBMCs) containing the immune cells were isolated by the density gradient method using Ficoll-paque. Briefly, equal volume of whole blood and PBS was mixed carefully by inversion, overlayed on Ficoll-paque followed by centrifugation at 400 g for 30 min at RT. The PBMCs were retrieved from the buffy layer (see Low and Wan Abas, Biomed Res Int, 2015: 239362 (2015)). PBMCs were stimulated with anti-CD3 and anti-CD28 until day 2 prior to transduction.

The NK-92 cell line was used in assessment of NK cell functions. 1×10⁶ NK-92 cells were grown in T75 flasks and stimulated with IL-2 (100 UT/ml) in RPMI media containing 10% FBS. The cells were maintained for one week by supplementing IL-2 (100 UI/ml) every 48 h.

A transgene encoding for a factor (e.g., a polypeptide) that redirects glucose metabolites is co-expressed in the same immune (NK and/or T) cell with an ACTR (see Table 7) or CAR (see Table 8-Table 11) polypeptide. The transgene is, for example, PKM2, PKM2 Y105E variant, PKM2 Y105D variant, PKM2 K422R variant, PKM2 H391Y variant, GFPT1, or TIGAR (e.g., SEQ ID NO: 68-SEQ ID NO: 74). The T and/or NK cells were transduced with a virus encoding the ACTR or CAR polypeptide and the factor that redirects glucose metabolites separated, for example, by a P2A ribosomal skip sequence. Briefly, 1×10⁶ cells were mixed with 1 ml of viral supernatant (see Example 1) in a total volume of 2 ml, centrifuged at 1200 g for 45 min followed by plating into a 24-well plate. The cells were then incubated at 37° C. in a 5% CO₂ incubator. In case of NK-92 transduced cells, the culture was monitored for growth every 48 hrs and split to a final concentration of 0.5×10⁶ cell/ml by supplementing IL-2 (100 UI/ml) every 48 hrs.

The transduced immune cells were assessed for the transgene expression by immunoblotting. The transduced cells (e.g., NK-92), were harvested by centrifuging at 1500 rpm for 5 min at RT. The supernatant was removed, and the cell pellet was washed twice in 1×PBS before flash freezing in liquid nitrogen and stored at −80° C. until further use. Cell pellets were subsequently lysed in 200 μl of SDS Lysis buffer (Cat #NP0008; Novex) containing 1×HALT Protease Inhibitor Cocktail (Cat #78430; Thermo Fisher) followed by sonication. The suspension was centrifuged at 15,000 rpm for 15 min at RT and the supernatant containing total protein was collected. The total protein concentration was measured using Pierce 660 nm Protein Assay (Cat #1861426; Thermo Scientific) followed by immunoblotting. 10 μg total protein was loaded in each lane of a Novex™ 4 to 12% Tris-Glycine Plus, 1.0 mm, 20-well Midi Protein Gel (Invitrogen), transferred onto PVDF membrane using Transblot Turbo (Biorad) and blocked for 1 h at RT using LICOR Blocking buffer. The membrane was probed for transgenes (e.g GOT2, TIGAR) using mouse α-Actin (3700S, CST; dilution 1:2000), Rabbit α-TIGAR (14751S CST; dilution 1:1000) and Rabbit α-Got2 (NBP232241, Novus; dilution 1:2000) antibodies overnight (in 0.1% Tween 20+LICOR Blocking buffer) at 4° C. The following day, membranes were washed thrice with 1×TBS containing 0.1% Tween20 detergent (w/v) for 5 min each. Membranes were subsequently incubated with standard rabbit or mouse secondary antibodies (LICOR; dilution 1:10,000) for 1 h. The membranes were washed thrice with 1×TBS containing 0.1% Tween20 detergent (w/v) for 5 min each. Immunoblots were imaged using a CLX imager (LICOR) and processed in the Image Studio Software (v5.2; LICOR).

### Example 17: Analysis of CAR Expression in Transduced Immune Cells

Immune cells (such as NK and/or T cells) were isolated either from fresh blood samples or are derived from cell lines and were transduced as described in Example 16. On day 7 post-transduction, the cells were harvested by centrifuging at 1500 rpm for 5 min at RT. The supernatant was removed, and the cell pellet was washed twice in 1×PBS followed by staining with Live Dead Aqua (Cat No. L34966; Thermo Fischer) for 10 min at RT. The cells were washed twice in 1×PBS followed by staining with primary and secondary antibody in 1×PBS with 2% FBS for assessing CAR expression. Living single cells were selected for and CAR expression was determined in comparison to an untransduced control (Null). Data were analyzed with FlowJo version 10.7.1 software (Tree Star Inc).

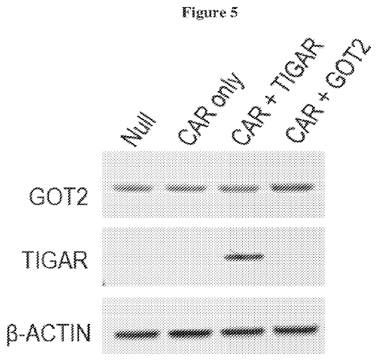

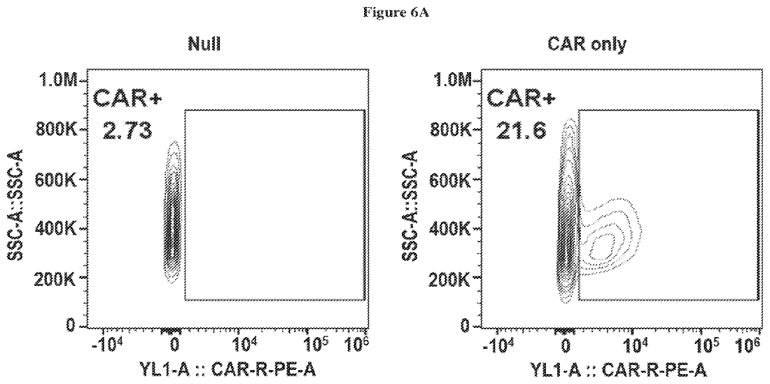

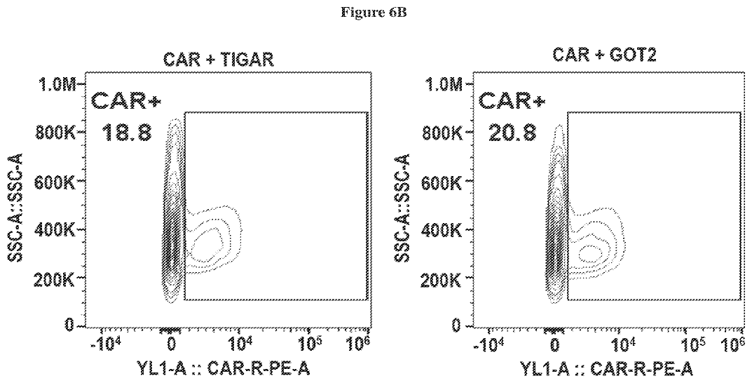

Co-expression of a CAR construct alone or together with TIGAR or GOT2 has been demonstrated in NK92 cells, an IL-2 dependent NK cell line derived from a patient with lymphoma. Transgene overexpression was analyzed for harvested cells on day 7 by immunoblotting (see FIG. 5). GOT2 expression was observed with all constructs and control due to the endogenous expression of GOT2, whereas a stronger band was observed in case of GOT2 co-expression with the CAR. No endogenous TIGAR expression was observed under these conditions. Strong expression was only seen once TIGAR was co-expressed with the CAR. In addition, harvested cells were stained with a recombinant antibody against the Fc part of the CAR, and presence of the CAR on the surface of the NK cell was assessed using flow cytometry. FIG. 6 shows that all constructs expressed the CAR on the surface of the NK92 cells.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of”. “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A genetically engineered immune cell, which has altered glucose metabolism as compared with a native immune cell of the same type, wherein the immune cell:
(i) expresses or overly expresses a polypeptide that diverts or redirects glucose metabolites out of a glycolysis pathway, and
(ii) a chimeric receptor polypeptide; wherein the chimeric receptor polypeptide comprises:
(a) an extracellular target binding domain;
(b) a transmembrane domain; and
(c) a cytoplasmic signaling domain.

2. The genetically engineered immune cell of claim 1, wherein the polypeptide that diverts or redirects glucose metabolites (i) is selected from the group consisting of pyruvate kinase muscle isozyme M2 (PKM2), glutamine-fructose-6-phosphate aminotransferase 1 (GFPT1), and TP53-inducible glycolysis and apoptosis regulator (TIGAR).

3. The genetically engineered immune cell of claim 2, wherein the polypeptide that diverts or redirects glucose metabolites is TIGAR.

4. The immune cell of claim 1, wherein the chimeric receptor polypeptide comprises one or more of the following features:
(i) the chimeric receptor polypeptide further comprises at least one co-stimulatory signaling domain or is free of co-stimulatory signaling domains;
(ii) the cytoplasmic signaling domain (c) comprises an immunoreceptor tyrosine-based activation motif (ITAM);
(iii) the cytoplasmic signaling domain (c) is located at the C-terminus of the chimeric receptor polypeptide;
(iv) the chimeric receptor polypeptide further comprises a hinge domain, which is located at the C-terminus of (a) and the N-terminus of (b),
(v) the chimeric receptor polypeptide is free of any hinge domain; and
(vi) the chimeric receptor polypeptide further comprises a signal peptide at its N-terminus.

5. The genetically engineered immune cell of claim 3, wherein the chimeric receptor polypeptide is preferably a chimeric receptor antigen (CAR) polypeptide, in which (ii)(a) is an extracellular antigen binding domain.

6. The genetically engineered immune cell of claim 5, wherein the extracellular antigen binding domain of (ii)(a) is a single chain variable fragment (scFv) or a single domain antibody that binds to a tumor antigen, a pathogenic antigen, or an immune cell specific to an autoantigen.

7. The genetically engineered immune cell of claim 6, wherein the extracellular antigen binding domain of (ii)(a) binds to the tumor antigen, which is associated with a hematologic or solid tumor.

8. The genetically engineered immune cell of claim 6, wherein the extracellular antigen binding domain of (ii)(a) binds to the pathogenic antigen, which is a bacterial antigen, a viral antigen, or a fungal antigen.

9. The genetically engineered immune cell of claim 1, wherein the transmembrane domain is of a membrane protein selected from the group consisting of CD8α, CD8β, 4-1BB, CD27, CD28, CD34, CD4, FcεRIγ, CD16A, OX40, CD3ζ, CD3ε, CD3γ, CD3δ, TCRα, TCRβ, TCRζ, CD32, CD64, CD45, CD5, CD9, CD22, CD37, CD80, CD86, CD40, CD40UCD154, VEGFR2, FAS, FGFR2B, CD2, IL15, IL15R, IL21, DNAM-1, 2B4, NKG2D, NKp44 and NKp46.

10. The genetically engineered immune cell of claim 1, wherein the chimeric receptor polypeptide comprises the at least one co-stimulatory signaling domain, which is of a co-stimulatory molecule selected from the group consisting of 4-1BB, CD28, CD8α, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL, or a functional variants thereof.

11. The genetically engineered immune cell of claim 1, wherein the at least one co-stimulatory signaling domains is a CD28 co-stimulatory signaling domain or a 4-1BB co-stimulatory signaling domain.

12. The genetically engineered immune cell of claim 1, wherein the chimeric receptor polypeptide comprises two co-stimulatory signaling domains.

13. The genetically engineered immune cell of claim 12, wherein
(i) one of the co-stimulatory signaling domains is a CD28 co-stimulatory signaling domain; and wherein the other co-stimulatory domain is selected from the group consisting of a CD8α, 4-1BB, 2B4, OX40, OX40L, ICOS, CD27, GITR, HVEM, TIM1, LFA 1, CD2, DAP10, DAP12, DNAM-1, NKG2D, NKp30, NKp44, NKp46 and JAMAL co-stimulatory signaling domain;
(ii) one of the co-stimulatory signaling domains is a CD8α co-stimulatory signaling domain; and wherein the other co-stimulatory domain is selected from the group consisting of a CD28, 4-1BB, 2B4, OX40, OX40L, ICOS, CD27, GITR, HVEM, TIM1, LFA 1, CD2, DAP10, DAP12, DNAM-1, NKG2D, NKp30, NKp44, NKp46 and JAMAL co-stimulatory signaling domain’ or
(iii) one of the co-stimulatory signaling domains is a 4-1BB co-stimulatory signaling domain; and wherein the other co-stimulatory domain is selected from the group consisting of a CD8α, CD28, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain.

14. The genetically engineered immune cell of claim 1, wherein the cytoplasmic signaling domain of (c) is a cytoplasmic domain of CD3ζ or FcεR1γ.

15. The genetically engineered immune cell of claim 1, wherein the chimeric receptor polypeptide comprises the hinge domain (iv), which is a hinge domain selected from the list of CD28, CD16A, CD8α, IgG, murine CD8α, and DAP12.

16. The genetically engineered immune cell of claim 1, wherein the immune cell is an αβ T, γδ T or a natural killer (NK) cell.

17. The genetically engineered immune cell of claim 16, wherein the immune cell is an αβ T cell, and wherein the chimeric receptor polypeptide is a CAR polypeptide that comprises components as shown in Table 8.

18. The genetically engineered immune cell of claim 16, wherein the immune cell is a NK cell, and wherein the chimeric receptor polypeptide is a CAR polypeptide that comprises components as shown in Table 9.

19. The genetically engineered immune cell of claim 16, wherein the immune cell is a γδ T cell, and wherein the chimeric receptor polypeptide is a CAR polypeptide that comprises components as shown in Table 10.

20. The genetically engineered immune cell of claim 1, wherein
(i) the immune cell is derived from cell lines; or
(ii) the immune cell is derived from peripheral blood mononuclear cells (PBMC), hematopoietic stem cells (HSCs), cord blood stem cells or induced pluripotent stem cells (iPSCs).

21. The genetically engineered immune cell of claim 1, wherein the immune cell comprises a nucleic acid or nucleic acid set, which collectively comprises:
(A) a first nucleotide sequence encoding the factor that diverts or redirects glucose metabolites; and
(B) a second nucleotide sequence encoding the chimeric receptor polypeptide.

22. The genetically engineered immune cell of claim 21, wherein the immune cell comprises the nucleic acid, which comprises both the first nucleotide sequence and the second nucleotide sequence.

23. The genetically engineered immune cell of claim 21, wherein the nucleic acid further comprises a third nucleotide sequence located between the first nucleotide sequence and the second nucleotide sequence, wherein the third nucleotide sequence encodes a ribosomal skipping site, an internal ribosome entry site (IRES), or a promoter.

24. The genetically engineered immune cell of claim 23, wherein the nucleic acid or nucleic acid set is comprised within one or more viral vectors.

25. A pharmaceutical composition, comprising a genetically engineered immune cell of claim 1.

26. A method for inhibiting cells expressing a target antigen in a subject, the method comprising administering to a subject in need thereof a population of the genetically engineered immune cells set forth in claim 1 or a pharmaceutical composition comprising the population of the genetically engineered immune cells.

27. The method for inhibiting cells expressing a target antigen in a subject of claim 26, wherein the subject is a human patient suffering from a cancer and the target antigen is a tumor antigen; wherein the cancer is selected from the group consisting of carcinoma, lymphoma, sarcoma, blastoma, and leukemia, preferably wherein
(i) the cancer is selected from the group consisting of a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, liver cancer, and thyroid cancer; or
(ii) the cancer of B-cell origin is selected from the group consisting of B-lineage acute lymphoblastic leukemia, B-cell chronic lymphocytic leukemia, and B-cell non-Hodgkin's lymphoma.

28. A nucleic acid or nucleic acid set, which collectively comprises:
(A) a first nucleotide sequence encoding a polypeptide that diverts or redirects glucose metabolites out of a glycolysis pathway; and
(B) a second nucleotide sequence encoding a chimeric receptor polypeptide set forth in claim 4.

29. A method for generating modified immune cells in vivo, the method comprising administering to a subject in need thereof the nucleic acid or nucleic acid set of claim 28.


SEQUENCE LISTING (85 sequences)

SEQ ID NO: 1 - protein, 21 aa, synthetic construct
MALPVTALLLPLALLLHAARP

SEQ ID NO: 2 - protein, 18 aa, synthetic construct
MLRLLLALNLFPSIQVTG

SEQ ID NO: 3 - protein, 20 aa, synthetic construct
METDTLLLWVLLLWVPGSTG

SEQ ID NO: 4 - protein, 44 aa, synthetic construct
TTTPAPRPPTPAPTIASQPLSLRPEACRPAAGGAVHTRGLDFAC

SEQ ID NO: 5 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 119 aa, synthetic construct
EPKSCDKTHTCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 12 aa, synthetic construct
EPKSCDKTHTCP

SEQ ID NO: 8 - protein, 12 aa, synthetic construct
ESKYGPPCPPCP

SEQ ID NO: 9 - protein, 29 aa, synthetic construct
TTTPAPRPPTPAPTIASQPLSLRPEAFAC

SEQ ID NO: 10 - protein, 14 aa, synthetic construct
TTTPAPRPPTPFAC

SEQ ID NO: 11 - protein, 39 aa, synthetic construct
IEVMYPPPYLDNEKSNGTIIHVKGKHLCPSPLFPGPSKP

SEQ ID NO: 12 - protein, 39 aa, synthetic construct; identical to SEQ ID NO: 11

SEQ ID NO: 13 - protein, 26 aa, synthetic construct
KSNGTIIHVKGKHLCPSPLFPGPSKP

SEQ ID NO: 14 - protein, 16 aa, synthetic construct
GKHLCPSPLFPGPSKP

SEQ ID NO: 15 - protein, 30 aa, synthetic construct
GGGGSGGGGSGGGGSGGGGSGGGGSGGGGS

SEQ ID NO: 16 - protein, 45 aa, synthetic construct
GGGGSGGGGSGGGGSGGGGSGGGGSGGGGSGGGGSGGGGSGGGGS

SEQ ID NO: 17 - protein, 60 aa, synthetic construct
GGGGSGGGGSGGGGSGGGGSGGGGSGGGGSGGGGSGGGGSGGGGSGGGGSGGGGSGGGGS

SEQ ID NO: 18 - protein, 25 aa, synthetic construct
DIYIWAPLAGTCGVLLLSLVITLYC

SEQ ID NO: 19 - protein, 28 aa, synthetic construct
DIISFFLALTSTALLFLLFFLTLRFSVV

SEQ ID NO: 20 - protein, 30 aa, synthetic construct
DFWVLVVVGGVLACYSLLVTVAFIIFWVRS

SEQ ID NO: 21 - protein, 25 aa, synthetic construct
DLIALVTSGALLAVLGITGYFLMNR

SEQ ID NO: 22 - protein, 25 aa, synthetic construct
DLLAALLALLAALLALLAALLARSK

SEQ ID NO: 23 - protein, 26 aa, synthetic construct
DMALIVLGGVAGLLLFIGLGIFFCVR

SEQ ID NO: 24 - protein, 25 aa, synthetic construct
DLCYILDAILFLYGIVLTLLYCRLK

SEQ ID NO: 25 - protein, 25 aa, synthetic construct
DLLLILLGVLAGVLATLAALLARSK

SEQ ID NO: 26 - protein, 25 aa, synthetic construct
DITLGLLVAGVLVLLVSLGVAIHLC

SEQ ID NO: 27 - protein, 25 aa, synthetic construct
DVSFCLVMVLLFAVDTGLYFSVKTN

SEQ ID NO: 28 - protein, 25 aa, synthetic construct
DVAAILGLGLVLGLLGPLAILLALY

SEQ ID NO: 29 - protein, 29 aa, synthetic construct
DVMSVATIVIVDICITGGLLLLVYYWSKN

SEQ ID NO: 30 - protein, 25 aa, synthetic construct
DLCYLLDGILFIYGVILTALFLRVK

SEQ ID NO: 31 - protein, 25 aa, synthetic construct
DGFLFAEIVSIFVLAVGVYFIAGQD

SEQ ID NO: 32 - protein, 26 aa, synthetic construct
DGIIVTDVIATLLLALGVFCFAGHET

SEQ ID NO: 33 - protein, 24 aa, synthetic construct
DVIGFRILLLKVAGFNLLMTLRLW

SEQ ID NO: 34 - protein, 27 aa, synthetic construct
DIIVAVVIATAVAAIVAAVVALIYCRK

SEQ ID NO: 35 - protein, 25 aa, synthetic construct
DVLFYLAVGIMFLVNTVLWVTIRKE

SEQ ID NO: 36 - protein, 25 aa, synthetic construct
DIIILVGTAVIAMFFWLLLVIILRT

SEQ ID NO: 37 - protein, 21 aa, synthetic construct
DLGWLCLLLLPIPLIVWVKRK

SEQ ID NO: 38 - protein, 25 aa, synthetic construct
DIAIYCIGVFLIACMVVTVILCRMK

SEQ ID NO: 39 - protein, 28 aa, synthetic construct
FACDIYIWAPLAGTCGVLLLSLVITLYC

SEQ ID NO: 40 - protein, 42 aa, synthetic construct
KRGRKKLLYIFKQPFMRPVQTTQEEDGCSCRFPEEEEGGCEL

SEQ ID NO: 41 - protein, 41 aa, synthetic construct
RSKRSRLLHSDYMNMTPRRPGPTRKHYQPYAPPRDFAAYRS

SEQ ID NO: 42 - protein, 42 aa, synthetic construct
ALYLLRRDQRLPPDAHKPPGGGSFRTPIQEEQADAHSTLAKI

SEQ ID NO: 43 - protein, 22 aa, synthetic construct
ERVQPLEENVGNAARPRFERNK

SEQ ID NO: 44 - protein, 34 aa, synthetic construct
KKKYSSSVHDPNGEYMFMRAVNTAKKSRLTDVTL

SEQ ID NO: 45 - protein, 48 aa, synthetic construct
QRRKYRSNKGESPVEPAEPCHYSCPREEEGSTIPIQEDYRKPEPACSP

SEQ ID NO: 46 - protein, 58 aa, synthetic construct
QLGLHIWQLRSQCMWPRETQLLLEVPPSTEDARSCQFPEEERGERSAEEKGRLGDLWV

SEQ ID NO: 47 - protein, 60 aa, synthetic construct
CVKRRKPRGDVVKVIVSVQRKRQEAEGEATVIEALQAPPDVTTVAVEETIPSFTGRSPNH

SEQ ID NO: 48 - protein, 48 aa, synthetic construct
KKYFFKKEVQQLSVSFSSLQIKALQNAVEKEVQAEDNIYIENSLYATD

SEQ ID NO: 49 - protein, 84 aa, synthetic construct
DIYIWAPLAGTCGVLLLSLVITLYCYKVGFFKRNLKEKMEAGRGVPNGIPAEDSEQLASGQEAGDPGCLKPLHEKDSESGGGKD

SEQ ID NO: 50 - protein, 141 aa, synthetic construct
DIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 51 - protein, 89 aa, synthetic construct
RSKRSRLLHSDYMNMTPRRPGPTRKHYQPYAPPRDFAAYRSQRRKYRSNKGESPVEPAEPCHYSCPREEEGSTIPIQEDYRKPEPACSP

SEQ ID NO: 52 - protein, 78 aa, synthetic construct
RSKRSRLLHSDYMNMTPRRPGPTRKHYQPYAPPRDFAAYRSRRDQRLPPDAHKPPGGGSFRTPIQEEQADAHSTLAKI

SEQ ID NO: 53 - protein, 83 aa, synthetic construct
KRGRKKLLYIFKQPFMRPVQTTQEEDGCSCRFPEEEEGGCELRSKRSRLLHSDYMNMTPRRPGPTRKHYQPYAPPRDFAAYRS

SEQ ID NO: 54 - protein, 80 aa, synthetic construct
RSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 55 - protein, 75 aa, synthetic construct
RSKRSRLLHSDYMNMTPRRPGPTRKHYQPYAPPRDFAAYRSKKKYSSSVHDPNGEYMFMRAVNTAKKSRLTDVTL

SEQ ID NO: 56 - protein, 41 aa, synthetic construct
RSKRSRGGHSDYMNMTPRRPGPTRKHYQPYAPPRDFAAYRS

SEQ ID NO: 57 - protein, 83 aa, synthetic construct
RSKRSRGGHSDYMNMTPRRPGPTRKHYQPYAPPRDFAAYRSKRGRKKLLYIFKQPFMRPVQTTQEEDGCSCRFPEEEEGGCEL

SEQ ID NO: 58 - protein, 23 aa, synthetic construct
CARPRRSPAQEDGKVYINMPGRG

SEQ ID NO: 59 - protein, 61 aa, synthetic construct
NRRRRRERRDLFTESWDTQKAPNNYRSPISTSQPTNQSMDDTREDIYVNYPTFSRRPKTRV

SEQ ID NO: 60 - protein, 45 aa, synthetic construct
GSTVYYQGKCLTWKGPRRQLPAVVPAPLPPPCGSSAHLLPPVPGG

SEQ ID NO: 61 - protein, 63 aa, synthetic construct
WWGDIWWKTMMELRSLDTQKATCHLQQVTDLPWTSVSSPVEREILYHTVARTKISDDDDEHTL

SEQ ID NO: 62 - protein, 28 aa, synthetic construct
GKPIPNPLLGLDSTGKPIPNPLLGLDST

SEQ ID NO: 63 - protein, 6 aa, synthetic construct
HHHHHH

SEQ ID NO: 64 - protein, 16 aa, synthetic construct
DYKDDDDKDYKDDDDK

SEQ ID NO: 65 - protein, 27 aa, synthetic construct
YPYDVPDYAYPYDVPDYAYPYDVPDYA

SEQ ID NO: 66 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 19 aa, synthetic construct
ATNFSLLKQAGDVEENPGP

SEQ ID NO: 68 - protein, 699 aa, synthetic construct
MCGIFAYLNYHVPRTRREILETLIKGLQRLEYRGYDSAGVGFDGGNDKDWEANACKIQLIKKKGKVKALDEEVHKQQDMDLDIEFDVHLGIAHTRWATHGEPSPVNSHPQRSDKNNEFIVIHNGIITNYKDLKKFLESKGYDFESETDTETIAKLVKYMYDNRESQDTSFTTLVERVIQQLEGAFALVFKSVHFPGQAVGTRRGSPLLIGVRSEHKLSTDHIPILYRTARTQIGSKFTRWGSQGERGKDKKGSCNLSRVDSTTCLFPVEEKAVEYYFASDASAVIEHTNRVIFLEDDDVAAVVDGRLSIHRIKRTAGDHPGRAVQTLQMELQQIMKGNFSSFMQKEIFEQPESVVNTMRGRVNFDDYTVNLGGLKDHIKEIQRCRRLILIACGTSYHAGVATRQVLEELTELPVMVELASDFLDRNTPVFRDDVCFFLSQSGETADTLMGLRYCKERGALTVGITNTVGSSISRETDCGVHINAGPEIGVASTKAYTSQFVSLVMFALMMCDDRISMQERRKEIMLGLKRLPDLIKEVLSMDDEIQKLATELYHQKSVLIMGRGYHYATCLEGALKIKEITYMHSEGILAGELKHGPLALVDKLMPVIMIIMRDHTYAKCQNALQQVVARQGRPVVICDKEDTETIKNTKRTIKVPHSVDCLQGILSVIPLQLLAFHLAVLRGYDVDFPRNLAKSVTVE

SEQ ID NO: 69 - protein, 270 aa, synthetic construct
MARFALTVVRHGETRFNKEKIIQGQGVDEPLSETGFKQAAAAGIFLNNVKFTHAFSSDLMRTKQTMHGILERSKFCKDMTVKYDSRLRERKYGVVEGKALSELRAMAKAAREECPVFTPPGGETLDQVKMRGIDFFEFLCQLILKEADQKEQFSQGSPSNCLETSLAEIFPLGKNHSSKVNSDSGIPGLAASVLVVSHGAYMRSLFDYFLTDLKCSLPATLSRSELMSVTPNTGMSLFIINFEEGREVKPTVQCICMNLQDHLNGLTETR

SEQ ID NO: 70 - protein, 531 aa, synthetic construct
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 71 - protein, 531 aa, synthetic construct
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILERPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 72 - protein, 531 aa, synthetic construct
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILDRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 73 - protein, 531 aa, synthetic construct
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFRCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 74 - protein, 531 aa, synthetic construct
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYYLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 75 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 76 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 77 - protein, 243 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 78 - protein, 487 aa, synthetic construct
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 483 aa, synthetic construct
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 492 aa, synthetic construct
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 81 - protein, 430 aa, synthetic construct
MALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 82 - protein, 18 aa, synthetic construct
EGRGSLLTCGDVEENPGP

SEQ ID NO: 83 - protein, 20 aa, synthetic construct
QCTNYALLKLAGDVESNPGP

SEQ ID NO: 84 - protein, 23 aa, synthetic construct
AVKQTLNFDLLKLAGDVESNPGP

SEQ ID NO: 85 - DNA, 463 bp, synthetic construct
  source 1..463: mol_type = other DNA
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa